(19) United States
(12) Patent Application Publication
(10) Pub. No.: US 2024/0402183 A1
(43) Pub. Date: Dec. 5, 2024

(54) SPATIAL METRIC MEASUREMENT METHODS FOR MULTIPLEX IMAGING AND USES IN TUMOR SAMPLE ANALYSIS

(71) Applicants: CEDARS-SINAI MEDICAL CENTER, Los Angeles, CA (US); BC CANCER, part of the Provincial Health Services Authority, Vancouver (CA)

(72) Inventors: Alexander Xu, Valley Village, CA (US); Akil Merchant, West Hills, CA (US); Anthony Colombo, Whittier, CA (US); Tomohiro Aoki, Vancouver (CA); Aixiang Jiang, Vancouver (CA); Christian Steidl, North Vancouver (CA)

(73) Assignees: CEDARS-SINAI MEDICAL CENTER, Los Angeles, CA (US); BC CANCER, part of the Provincial Health Services Authority, Vancouver (CA)

(21) Appl. No.: 18/680,481

(22) Filed: May 31, 2024

Related U.S. Application Data
(60) Provisional application No. 63/470,740, filed on Jun. 2, 2023.

(51) Int. Cl. G01N 33/574; C12Q 1/6874; C12Q 1/6886

(52) U.S. Cl. CPC G01N 33/57492; C12Q 1/6874; C12Q 1/6886

(57) ABSTRACT

Spatial analysis methods based on nearest neighbor analysis are provided of specific regions of tissue to describe cancer states and develop biomarkers to predict outcomes. In various embodiments, the spatial analysis methods involve an interaction radius to computationally isolate local interactions and prevent the data from being influenced by cells that are very far away. Thereby, spatial analysis of cancer tissue can be quickly and consistently performed in an automated fashion to focus on tumor-immune cell biology, which occurs at a localized level.

### CROSS-REFERENCE TO RELATED APPLICATIONS

This application includes a claim of priority under 35 U.S.C. § 119 (e) to U.S. provisional patent application No. 63/470,740, filed Jun. 2, 2023, the entirety of which is hereby incorporated by reference.

### STATEMENT REGARDING FEDERALLY SPONSORED RESEARCH OR DEVELOPMENT

This invention was made with Government support under grant nos. CA266544 and TR001882 awarded by the National Institutes of Health. The Government has certain rights in the invention.

### FIELD OF INVENTION

This invention relates to spatial analysis of regions of tissue to describe cancer states and develop biomarkers to predict outcomes.

### BACKGROUND

Multiplexed spatial protein data has the potential to reveal cell-cell interactions at a local level in tissue, which is highly relevant to tumor immunology. The technology to generate this type of data has only recently become available. Other pipelines to analyze this type of data use different types of spatial analyses, for example, graph-based approaches or nearest neighbor approaches. However, a significant challenge exists with the overwhelming amount of data and analysis possible in spatial imaging data.

For example, treatment of classical HL is widely regarded as a model of success, with chemotherapy having greatly improved patient survival. Despite these improvements, a proportion of patients with advanced-stage disease either harbor refractory lymphoma (10%) or experience relapse after first-line treatment (20% to 30%). The current standard of care for young, fit patients who experience refractory or relapsed disease is salvage chemotherapy followed by high-dose chemotherapy and autologous stem-cell transplantation (ASCT). Approximately 50% of patients are not cured by such therapy and eventually die as a result of the disease.

Therefore, it is an object of the present invention to provide a simpler method to process multiplexed spatial protein data to, for example, clinicians and researchers.

It is also an object of the present invention to provide methods to stratify patients having refractory or relapsed HL and provide prognosis of outcome of salvage chemotherapy followed by high-dose chemotherapy and autologous stem-cell transplantation (ASCT), e.g., no (or late) relapse v. early relapse. It is another object of the present invention to identify and provide treatment to patients at high-risk of treatment failure such as early relapse. It is another object of the present invention to guide/provide treatment decisions for patients having refractory or relapsed HL at the time of first treatment failure (i.e., occurrence of refractory or relapsed HL) and before initiation of salvage therapy.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods of computing an enrichment score for a target cell (also called a home cell) in a tissue sample, the enrichment score representing enrichment of a selected cell type (also called an enriching cell type) around the target cell (the home cell), the method comprising:
a. measuring a distance from the target cell to its nearest cell or to each one of its nearest two or more cells of the selected cell type within a predetermined radius, optionally via an image of the tissue sample;
b. scaling the measured distance via division by the predetermined radius, thereby obtaining a scaled distance, and if the measuring in step a comprises measuring the distance to each one of the nearest two or more cells, then further averaging the scaled distance to obtain an average scaled distance; and
c. performing an inverse operation on the scaled distance or the average scaled distance, thereby obtaining an inverted scaled distance from the target cell to its nearest cell or nearest two or more cells of the selected cell type as the enrichment score for the target cell; and
d. optionally displaying, on a computer screen, the enrichment score based on the inverted scaled distance.

In some embodiments, the computation further includes performing one or more operations based on the enrichment scores, including but are not limited to clustering (such as k-means clustering, phenography).

In some embodiments, the tissue sample comprises a quantity of discretely located cells of a same cell type as the target cells, and the method further comprises repeating steps a-c to calculate an enrichment score for each one of the target cells.

In some embodiments, the tissue sample in the image comprises two or more different cell types around the target cell, and the method further comprises repeating steps a-c to calculate an enrichment score representing enrichment of each one of the two or more different cell types around the target cell.

In some embodiments, the target cell is of a same cell type as the selected cell type, thereby the enrichment score representing clustering of the same target cell type. In further embodiments, the tissue sample comprises two or more different cell types, and the method further comprises repeating steps a-c to calculate an enrichment score representing clustering of each one of the two or more different cell types.

In some embodiments, the target cell (aka the home cell) is a tumor cell. In some embodiments, the tissue sample has a quantity of the tumor cell. In some embodiments, the tumor cell or the tissue sample is obtained from a subject with classical Hodgkin lymphoma. In some embodiments, the method further comprises repeating steps a-c to obtain an enrichment score for each tumor cell. In some embodiments, enrichment scores are computed for HRS tumor cell(s) as the home cell with PD-1+CD4+ T cells, CD68+ macrophages, CXCR5+ B cells, and CXCR5+ tumor cells as the enriching cell type individually.

In some embodiments, the method further includes applying a K-means clustering model by a processor based on data records containing the enrichment score for each of the different cell types to generate output data defining niches of the different cell types in the tissue sample; optionally wherein the data records exclude enrichment score greater than a first selected cutoff value, the data records exclude enrichment score smaller than a second selected cutoff value, or the data records exclude enrichment score greater than the first selected cutoff value and enrichment score smaller than the second selected cutoff value, wherein the second selected cutoff value is smaller than the first selected cutoff value.

In some embodiments, the nearest two or more cells comprise about five nearest cells of the selected cell type to the target cell, and the predetermined radius is about 50 μm.

In some embodiments, the inverse operation comprises subtracting the scaled distance or the average scaled distance from a fixed number, optionally the fixed number being 1 or 100%.

In some embodiments, the inverse operation comprises dividing a fixed number by the scaled distance or the average scaled distance, optionally the inverse operation being configured for calculating a multiplicative inverse of the scaled distance or the average scaled distance.

In some embodiments, the measurement is performed on a mass cytometry image, a multicolor immunofluorescence image, or an immunohistochemical stained image, of the tissue sample.

In some embodiments, methods of providing enrichment scores further comprise one or more of:
i) measuring expression level of a first marker protein in the target cell, optionally further deriving a mathematical relation of the expression level of the first marker protein as a function of the total cell number of the selected cell type within the predetermined radius;
ii) measuring expression level of a second marker protein in the selected cell type within the predetermined radius; and
iii) computing a mathematical product of the first marker protein expression level in the target cell and the second marker protein expression level in the selected cell type.

In some embodiments, the tissue sample has been treated with a panel of labeled antibodies against at least 5, 10, 20, 30, 40, 50, or more marker proteins in the tissue sample, and the method further comprises measuring label intensities of one or more of the at least 5, 10, 20, 30, 40, 50, or more marker proteins in the tissue sample.

Various embodiments provide systems for performing spatial metric analysis including calculating an enrichment score representing enrichment of a selected cell type around a target cell (aka a home cell) in a tissue sample, the systems comprising:
a processor operable to execute computer executable instructions;
a memory operable to store computer executable instructions executable by the processor; and
computer executable instructions stored in the memory and executable to perform the steps in one or more methods disclosed herein for computing enrichment scores.

Various embodiments provide a non-transient computer readable medium, which includes computer executable instructions, recorded on the non-transient computer readable medium, executable by a processor, for performing the steps in one or more methods disclosed herein for computing enrichment scores to perform spatial metric analysis including calculating an enrichment score representing enrichment of a selected cell type around a target cell in a tissue sample.

Various embodiments provide methods for treating refractory or relapsed classical Hodgkin lymphoma (cHL) in a human subject, the method comprising:
providing a salvage therapy comprising autologous stem cell transplantation (ASCT) or a combination of high-dose chemotherapy and the ASCT to the human subject if the human subject is detected in a biopsy sample of the human subject with presence of enrichment of CXCR5+ B cells around a Hodgkin and Reed Sternberg (HRS) tumor cell and with absence of CXCR5+ HRS tumor cells and absence of enrichment of CXCL13+ macrophages or PD-1+CD4+ T cells around the CXCR5+ HRS tumor cells; or
providing allogeneic bone marrow transplantation, a CD30 targeting treatment, and/or brentuximab vedotin to the human subject if the human subject is detected in the biopsy sample with presence of the CXCR5+ HRS tumor cells and enrichment of the CXCL13+ macrophages and/or PD-1+CD4+ T cells around the CXCR5+ HRS tumor cells;
wherein the enrichment of CXCR5+ B cells comprises two or more CXCR5+ B cells within a radius of no more than about 50 μm from the HRS tumor cell, and the enrichment of CXCL13+ macrophages and/or PD-1+CD4+ T cells comprises two or more of CXCL13+ macrophages and/or PD-1+CD4+ T cells within the radius from the CXCR5+ HRS tumor cells; and
wherein the high-dose chemotherapy comprises a higher dose of chemotherapy than that of a prior chemotherapy to which the cHL is refractory or has relapsed.

Various embodiments provide methods for treating a refractory or relapsed classical Hodgkin lymphoma (r/r cHL) in a human subject, the method comprising:
(a) obtaining two or more enrichment scores for a target cell (also called a home cell) in a biopsy sample from the human subject, each enrichment score representing enrichment of a selected cell type (also called an enriching cell type) around the target cell (also called home cell), wherein the target cell (that is, the home cell) comprises a Hodgkin and Reed Sternberg (HRS) tumor cell, and wherein the selected cell types (that is, the enriching cell types) comprise CXCR5+ HRS tumor cells, PD1+CD4+ T cells, CD68+ macrophages, and CXCR5+ B cells, and wherein each enrichment score is an inverse of an average of scaled distances from the target cell (that is, the home cell) to its nearest two or more cells of respective selected cell type within a predetermined radius, such that the inverse results in a greater enrichment score for a smaller averaged scaled distance compared to that for a larger averaged scaled distance;
(b) calculating a linear predictor score (LPS) for the biopsy sample, wherein each LPS is a linear, weighted combination of the enrichment scores representing enrichment of the four different selected cell types calculated from (a), using an equation:

$$LPS ( X ) = {\sum }_{j} {a}_{j} {X}_{j}$$

wherein Xⱼ is the enrichment score representing enrichment of a selected cell type j around the target cell or is a mean, median or average of top 10%, 20%, 30%, 40%, 50%, 60%, 70%, 80%, or 90% of the highest enrichment scores calculated for a plurality of the target cell type in the biopsy sample; and aⱼ is a scaling factor or weight for the selected cell type j, optionally aⱼ being within 0 to 1; and
(c) calculating a probability that the human subject is at low-risk or at high-risk of salvage treatment failure, optionally salvage treatment failure comprising further relapse (or disease-related death) within 1 year, 2 years, 3 years, 4 years, 5 years, or a user-defined future, using an equation:

$$P ( X ) = \frac{ϕ ( LPS ( X ) ; {μ}_{1} , {σ}_{1}^{2} )}{ϕ ( LPS ( X ) ; {μ}_{1} , {σ}_{1}^{2} ) + ϕ ( LPS ( X ) ; {μ}_{2} , {σ}_{2}^{2} )} ,$$

wherein:
   for calculating the probability of low-risk of salvage treatment failure, Ø(LPS(X);μ₁, σ₁²) is a Phi value when the calculated LPS from step (b) is applied in a normal distribution function with mean μ₁ and variance σ₁² from a first r/r cHL patient population known to have no salvage therapy failure or known with low-risk of salvage therapy failure, and Ø(LPS(X); μ₂,σ₂²) is a Phi value when the calculated LPS from step (b) is applied in a normal distribution function with mean μ₂ and variance σ₂² from a second r/r cHL patient population with known salvage therapy failure or known high-risk of salvage therapy failure; or
   for calculating the probability of high-risk of salvage treatment failure, Ø(LPS(X);μ₁, σ₁²) is a Phi value when the calculated LPS from step (b) is applied in a normal distribution function with mean μ₁ and variance σ₁² from the second r/r cHL patient population with known salvage therapy failure or known high-risk of salvage therapy failure, and Ø(LPS(X);μ₂,σ₂²) is a Phi value when the calculated LPS from step (b) is applied in a normal distribution function with mean μ₂ and variance σ₂² from the first r/r cHL patient population known to have no salvage therapy failure or known with low-risk of salvage therapy failure;
(d) classifying the human subject as at low-risk of salvage treatment failure if:
   the calculated probability of low-risk of salvage treatment failure from step (c) is 0.8 or greater (preferably), 0.9 or greater, 0.7 or greater, 0.6 or greater, 0.5 or greater, or
   the calculated probability of high-risk of salvage treatment failure from step (c) is less than 0.5, less than 0.4, less than 0.3, or less than 0.2;
   or
   classifying the human subject as at high-risk of salvage treatment failure if:
   the calculated probability of high-risk of salvage treatment failure from step (c) is 0.8 or greater (preferably), 0.9 or greater, 0.7 or greater, 0.6 or greater, 0.5 or greater, or
   the calculated probability of low-risk of salvage treatment failure from step (c) is less than 0.5, less than 0.4, less than 0.3, or less than 0.2; and
(e) providing treatment to the human subject, wherein the treatment comprises salvage therapy comprising autologous stem cell transplantation (ASCT) or a combination of high-dose chemotherapy and the ASCT if the human subject is indicated as at low-risk of salvage treatment failure, or wherein the treatment comprises allogeneic bone marrow transplantation, a CD30 targeting treatment, and/or brentuximab vedotin if the human subject is indicated as at high-risk of the salvage treatment failure.

In preferable embodiments, the r/r cHL treatment methods are performed by calculating the probability of low-risk of salvage treatment failure. In preferable embodiments, the subject is indicated as having low-risk of salvage treatment failure if the calculated probability of low-risk of salvage treatment failure is 0.8 or greater.

In some embodiments, the scaling factor or weight is a t-value derived from t-statistic of a generalized linear model of binary high/low risk stratification as a function of the enrichment score Xⱼ.

Various embodiments provide methods of treating a patient with Hodgkin's lymphoma, comprising:
administering a salvage therapy optionally a high dose chemotherapy to a patient who is detected with a lower enrichment score for tumor cells enriched with rosetting cells of CD8+ T cells and/or B cells in an image of a cancer tissue sample obtained from the patient, relative to that of a control subject who has relapsed later than 1 year or has no relapse,
who is detected with a co-expression pattern of marker proteins in the target tumor cell and in the rosetting cells, the co-expression pattern comprising:
   the target tumor cell being positive for inducible costimulatory ligand (ICOSL) and rosetting macrophages being positive for inducible T cell co-stimulator (ICOS),
   the target tumor cell being positive for galectin-9 and rosetting B cells and/or rosetting macrophages being positive for T cell immunoglobulin and mucin domain-containing protein 3 (TIM3), and/or
   the target tumor cell being positive for galectin-9 and rosetting CD4+ T cells, rosetting CD8+ T cells, rosetting B cells, and/or rosetting macrophages being positive for V-domain Ig suppressor of T cell activation (VISTA), and/or
who is detected with a higher CXCR5 expression level in the target tumor cell and/or in the rosetting cells in the cancer tissue sample obtained from the patient, relative to that of the control subject who has relapsed later than 1 year or has no relapse,
optionally with understanding that any one or more of said detections indicates that the patient is likely to have poor outcome or early relapse within 1 year from initial treatment against the Hodgkin's lymphoma.

Various embodiments provide methods of treating a patient with ovarian cancer, comprising: administering a therapy optionally chemotherapy against the ovarian cancer to a patient who is detected with a higher enrichment score for stromal cells surrounded by a same type of stromal cells, optionally the stromal cell being an immune cell or podoplanin-positive fibroblast, based on an image of a cancer tissue sample obtained from the patient, relative to that of a control subject who has relapsed after 15 months following debulking surgery for ovarian cancer, and/or who is detected with a higher percentage of B cells in a region with the higher enrichment score for the fibroblasts in the cancer tissue sample obtained at primary tumor stage from the patient, relative to that in a cancer tissue sample obtained at tumor recurrence stage from the patient. In various aspects, understanding is that either or both of said detections indicates the patient is likely to have relapsed ovarian cancer within 15 months following a debulking surgery for the ovarian cancer.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIGS. 1A-1C depict imaging mass cytometry (IMC) experimental design in Example 7. FIG. 1A. Consort diagram showing relapse status (non-relapse, late relapse, early relapse) and timepoint (diagnostic and relapse). FIG. 1B. A panel of 35 antibodies was used to image tissue. Antibodies were organized into three tiers to identify major cell phenotypes, their subtypes, and functional subsets of cell phenotypes. FIG. 1C. Sample images for an ROI are shown, showing the H&E and inset image (top left), segmentation results using ilastik (top middle), tumor and general immune phenotypes (top right), myeloid and dendritic cell markers (lower left), lymphocyte markers (lower middle), and markers indicating immune cell function (lower right). Scale bar-25 μm.

FIGS. 2A-2G depict HL phenotyping. FIG. 2A. A total of 10 major cell types were identified, with their mean expression heatmap and absolute proportions displayed. Cells that were unable to be classified within any of the categories were labeled “Unknown”. FIG. 2B. A UMAP was generated of all cells with major cell types colored. Unknown cells were often found between clusters. FIG. 2C. Total relative proportions of all cell types are shown. FIG. 2D. Sample strategy for clustering within major cell types is shown using 5 macrophage subtypes. The subtypes express combinations of CD163, Galectin9, and CD80, labeled 1-5. FIG. 2E. The coexpression patterns and relative proportions of macrophage subtypes are shown with subtypes 1-5 from FIG. 2D highlighted. FIG. 2F. Coexpression pattern of HRS cell subtypes. FIG. 2G. Coexpression pattern of CD4+ T cell subtypes.

FIGS. 3A-31 depict spatial architecture of HL. FIG. 3A. The average spatial enrichment of each cell type for every other cell type. Spatial enrichments were not reciprocal due to differences in their abundance, i.e. rare pDCs were spatially enriched with CD4+ T cells but not vice versa. FIG. 3B. Sample image of “digital biopsies” using thresholds of 0.5 and 10 μm for HRS spatial enrichment and nearest neighbor (NN) distance measurements, and cell proportions of tumor-enriched and tumor-contact regions. FIG. 3C. Using k-means clustering of spatial enrichment measurements with k=15, spatial cell niches defined. FIG. 3D. The frequency and cell-type specific expression of select markers in HRS, CD4+, CD8+ T cells, macrophages and cDCs in each of the 15 spatial niches. FIG. 3E. Aggregation of specific immune cells around HRS cells was seen at different extents, from 0 aggregating cells to 7+ aggregating cells. The aggregate size was defined as the number of cells with <15 μm centroid-to-centroid distance relative to a central cell. FIG. 3F. The ligand receptor co-expression of HRS cells and aggregating immune cells is shown as a function of the number of aggregating cells, and the strength and direction of the correlation varies. In patient 1, HRS TIM3, average CD4+ T cell Galectin9 expression, and their co-expression product are independent of the number of aggregating CD4+ T cells. In patient 2, the ligand-receptor pair co-expression is inversely correlated with aggregate size. FIG. 3G. Ligand receptor pair co-expression was measured and used to model rosette size for CD4+ and CD8+ T cells, with significant (p<0.05) measurements highlighted and colored by the direction of the association. FIG. 3H. The average coexpression of HRS-TIM3/CD4-Galectin9 is strongly associated with aggregate size in relapsed tumor samples, but not diagnostic samples. FIG. 3I. The significance of ligand-receptor coexpression measurements associated with aggregate size in diagnostic/relapse samples shows a distinct pattern for each cell type.

FIGS. 4A-4G depict biomarker testing and validation. FIG. 4A. Kaplan-Meier (KM) survival curve for CD68+ cell proportion and 4 sample images of CD68+ low and high samples. Scale bar-100 μm. FIG. 4B-4D. KM curves for GranzymeB+CD8+ T cells (4B), PDL1+ HRS cells (4C), and MHC-HRS cells (4D). FIG. 4E. The fraction of LAG3+ Tregs out of all Tregs was used as a biomarker in all cells or one of two digital biopsies (tumor region—HRS spatial enrichment >0.5, tumor contact—HRS nearest neighbor distance <10 μm). KM curves for the digital biopsies are compared, with a significance survival difference observed in all cells but neither biopsy. The relative significance of biomarker candidates in tumor region or tumor contact digital biopsies relative to the whole image is shown in diagnostic samples predicting overall survival. Biomarkers fall in one of 4 quadrants, with HRS-based biomarkers found in the bottom left (more significant in whole images), some immune biomarkers in the top right (more significant in tumor-enriched biopsies). Color indicates the product of p-values across the three spatial categories (all cells, tumor region, tumor contact, −log 10). FIG. 4F. Receptor-ligand coexpression patterns associated with aggregate size in diagnostic samples by relapse status. FIG. 4G. HRS-PD1/CD8-PDL1 coexpression vs. CD8+ T cell aggregate size by relapse status, with a statistically significant association in early relapse patients only.

FIGS. 5A-5F depict biomarker discovery with LASSO_plus. FIG. 5A. Biomarker candidate set of cell proportions identified using diagnostic samples with LASSO_plus. FIG. 5B. Biomarker candidate set identified using tumor region digital biopsy. FIG. 5C. Combined protein and spatial biomarker candidate set. FIG. 5D. Waterfall plot showing the type of biomarker candidate vs. the average significance of the candidate relative to p=0.05 for all major cell types, digital biopsies, timepoints, and survival conditions. Frequently appearing cell type proportions, proteins, and spatial measurements are highlighted. FIG. 5E. Waterfall plot for biomarker candidates generated to predict MHCII status, with common HLADPDQDR-related candidates as well as proteins such as IDO. FIG. 5F. Among spatial biomarker candidates, the frequency of LASSO_plus selection of spatial biomarkers is plotted as a function of cell type, showing the predictive power of specific cell-type spatial relationships.

FIG. 6 depicts prognostic protein expression patterns. Considering whole TMA-sized ROIs, classically recognized cell types such as CD163+ macrophages predicted early relapse and poor survival. Using spatial subsets of images via digital biopsies, additional macrophage and HRS protein expression patterns were prognostic. In ligand/receptor aggregation analysis, T cell and macrophage checkpoint patterns were significantly associated with early relapses.

FIGS. 7A-7B depict IMC pipeline and summary in relation to Example 7. FIG. 7A. For each ROI obtained from the TMA, clinical data such as patient outcomes and single cell data were recorded. Outcomes were stored as binary or categorical classes (EBV status, +/−1 year relapse), or survival. The antibody panel was designed for hierarchical clustering using lineage markers into major cell phenotypes (Tier 1), followed by additional clustering into cell subtypes (Tier 2) and functional subtypes (Tier 3). Cell classifications generated using the first or second clustering steps were developed into biomarker candidates. Single cell data were generated by segmentation using the ilastik pipeline, with edge cells during spatial analysis as having biased spatial patterns. FIG. 7B. Following data scaling, mean expression of each marker per ROI was approximately normally distributed.

FIGS. 8A-8G depict phenotyping pipeline. FIG. 8A. A hierarchical clustering pipeline was used to perform cell phenotyping and extract cell types and subtypes of interest. Both automated clustering steps (dashed boxes) and manual annotations (dotted boxes) were used. Depending on the complexity of the data, a two-step metaclustering step can be used initially. The results of the pipeline are major cell type and cell subtype labels assigned to each cell, with multiple labels allowed per cell and ambiguous cells labeled “Unknown”. FIG. 8B. A heatmap of the first clusters generated and their proportions. Entropy describes the diversity of patients containing each of the clusters, with low cluster entropy indicating that fewer patient samples contained cells of that cluster. FIG. 8C. Z-score transformation of mean cluster expression was used to automatically label cell types.

FIG. 9. A single clustering step of all relevant proteins generated similar clusters as the metaclustering approach.

FIGS. 10A-10I depict spatial protein expression. FIG. 10A. A spatial enrichment metric was calculated using the 5-nearest neighbor average distance, modified by capping at 50 μm to remove distant cell effects. Nearest neighbor distance was similarly capped at 50 μm. Spatial data was treated similarly to protein data to generate clusters of cells with similar spatial environments by clustering, or to select cells based on thresholded spatial measurements. FIG. 10B. A correlation plot shows the relative enrichments between all cell types. FIG. 10C. The within-cluster and between-cluster distances for k-means clustering, with no clear elbow. FIG. 10D. The number of HRS cells found with each aggregate size for CD4+, CD8+, Treg, B, and macrophage cells, and sample images of large aggregates of each type. FIG. 10E. A plot of the number of aggregates in an ROI vs the test fraction of random replacement tests with more aggregates than expected. FIG. 10F. The histogram of test fractions among ROIs, with ROIs with no aggregates observed highlighted. FIG. 10G. Receptor-ligand coexpression measurements associated with aggregate size in relapse samples, with differences from diagnostic samples indicated by +. FIG. 10H. Mean expression of proteins on aggregating cells associated with aggregate size in diagnostic and relapse samples. FIG. 10I. Expression of proteins on HRS cells associated with aggregate size in diagnostic and relapse samples.

FIG. 11A. Single cell biomarker candidate comparisons, D=Diagnostic, R=Relapse. FIG. 11B-11C. Relapse status-dependent protein expression associated with aggregate size on aggregating cells (11B) or HRS cells (11C) in diagnostic samples. FIG. 11D-11F. Differences observed in receptor-ligand coexpression (11D), aggregating cell protein (11E), and HRS cell (11F) protein expression between early and late relapses in relapse samples.

FIGS. 12A-12E depict biomarker discovery. FIG. 12A. LASSO_plus output of biomarker candidate set of cell proportions for relapse samples with zoom in inset. FIG. 12B. Biomarker candidate set for relapse samples in tumor contact digital biopsy. FIG. 12C. Biomarker candidate set of protein and spatial measurements of HRS cells. FIG. 12D. Biomarker candidate set for proteins and spatial measurements on macrophages in diagnostic samples. FIG. 12E. Biomarker meta-analysis was performed using LASSO_plus on combinations of biomarker types, spatial, temporal, and clinical conditions. For each biomarker candidate, the number of times it was selected by LASSO_plus was compared to the maximum possible times it could be selected to obtain the biomarker relative frequency. The geometric mean of p-values was also calculated.

FIGS. 13A-13F. Distinct spatially resolved tumor microenvironment features according to relapse status. FIG. 13A. Proportion for the indicated immune cell population by imaging mass cytometry (IMC)-based cluster assignment datasets. FIG. 13B. The alluvial plot shows the tumor-microenvironment (TME) types and their dynamic change between diagnostic samples and relapse samples according to relapse status. Horizontal ribbons represent individual cases and can be followed from left to right. Blue color of the ribbons indicates that there is no TME type change between diagnostic and relapse samples while red colored samples indicate change of TME type. FIG. 13C. Violin plot indicating the spatial score for the indicated cell types near Hodgkin and Reed Sternberg (HRS) cells according to relapse status. FIG. 13D. IMC analysis from FFPE sections of classic Hodgkin lymphoma (CHL) shows localization of immune cells according to relapse status. A representative case with early relapse CHL case (left) shows numerous CD163+ macrophage/myeloid cells and rare B cells. In contrast, a representative late relapse HL case (right) shows few CD163+ macrophage/myeloid cells and abundant B cells. FIG. 13E. Box plot indicating the spatial scores of macrophage/myeloid cell subtypes near Hodgkin and Reed Sternberg (HRS) cells according to relapse status. FIG. 13F. Dot plot showing correlation of spatial scores of major immune cell markers by imaging mass cytometry (IMC). Dot size and color summarize Pearson correlation values, with positive correlations represented in red and negative correlations represented in blue. Asterisks represent associated p-values (*P<0.05; **P<0.01; ***P<0.001).

FIGS. 14A-14E. Characteristics of the tumor-microenvironment of classic Hodgkin lymphoma (CHL) associated with CXCR5 positivity on HRS cells. FIG. 14A. Forest plots summarize the prognostic factors in relapsed classic Hodgkin lymphoma treated with HDC/ASCT according to HRS cells features by imaging mass cytometry (IMC). FIG. 14B. IHC staining for CXCR5 in representative cases with either positive (Left) or negative (Right) HRS cells (×400). FIG. 14C. The expression of CXCR5 in microdissected HRS cells from primary CHL samples (separated by CXCR5 status evaluated by IHC) and germinal center cells from reactive tonsil tissue (GCB; t test; ns: P>0.05; *, P≤0.05; **, P≤0.01). FIG. 14D. IMC image for selected immune subsets in representative cases with either CXCR5 positive (Left) or negative (Right) HRS cells. FIG. 14E. Relative proportion of cell subtypes near either negative (Left) or positive (Right) HRS cells. *P<0.05.

FIGS. 15A-15E. CXCL13/CXCR5 interaction in CHL. FIG. 15A. The dot plot shows significant ligand and receptor interaction between HRS cells (receptor) and immune cell populations (ligand) using Cell Chat. FIG. 15B. An interaction between CXCL13 and CXCR5 on immune cells and HRS cells in CHL samples was predicted using the iTALK tool. FIG. 15C. Dot plot showing correlations of the proportions of selected immune cell subsets with emphasis on CXCL13/CXCR5 interaction (MC-IHC). Dot size and color summarize Pearson correlation values, with positive correlations represented in blue and negative correlations represented in red. Asterisks represent associated p-values (*P<0.05; **P<0.01; ***P<0.001). FIG. 15D. Boxplot showing the spatial score of CXCL13+ and CXCL13-macrophages in the region surrounding CD30+ cells (HRS). FIG. 15E. Membrane map depicting CD68+CXCL13+ macrophages (light blue) and CD30+CXCR5+ HRS cells (red).

FIGS. 16A-16D. Development of a novel prognostic model, RHL4S, which predicts failure free survival after autologous stem cell transplantation (ASCT). FIG. 16A. Forest plots summarize the prognostic factors in relapsed classic Hodgkin lymphoma treated with HDC/ASCT according to imaging mass cytometry (IMC). FIG. 16B. Heatmap of the spatial scores in RHL4S according to IMC. Cases are ordered by RHL4S model score. Kaplan-Meier curves of the high-versus low-risk groups for (FIG. 16C) post-ASCT failure-free survival (FFS) and (FIG. 16D) post-ASCT overall survival (OS) as identified by RHL4S. P values were calculated using a log rank test.

FIGS. 17A-17B. Validation of RHL4S in independent cohort of relapsed and refractory classic Hodgkin Lymphoma (r/r CHL). Kaplan-Meier curves of the high-versus low-risk groups for (17A) post-ASCT failure-free survival (FFS) and (17B) post-ASCT overall survival (OS) as identified by RHL4S in the independent validation cohort, respectively. P values were calculated using a log rank test.

FIG. 18. Graphical summary of findings in relapsed and refractory classic Hodgkin Lymphoma (r/r CHL). r/r CHL with poor prognosis is characterized by CXCR5 positivity on HRS cells. CXCL13+ macrophages surround CXCR5+ HRS cells and PD1+CD4+ T cells were also present in the tumor-microenvironment. In contrast, CXCR5+ B cells were enriched in r/r CHL with good prognosis.

FIG. 19. Cohort and study design overview of Example 18. We analyzed IMC data from 164 CHL samples, including 71 patients with paired primary relapse specimens and 22 diagnostic control samples without any relapse. Subsequently, we developed a novel predictive model using spatial information. The predictive model was validated using an independent validation cohort of 44 patients.

FIG. 20. Scheme of spatial score. Scaled and inverted average distance to 5 nearest neighbors within interaction radius r from cells of interests (home cells) is calculated and scored. Representative images of regions with CD4 T cell (CD4) enrichment (center, green) and macrophage (Mac, blue) abundance (right) with HRS cell (Red) are shown at the bottom.

FIG. 21. Tumor-microenvironment subtype. Heatmap summarizing the enrichment of selected immune cell subtypes and HRS cells clustered using the Phenograph algorithm defined by Imaging Mass Cytometry data. Six tumor-microenvironment subtypes were identified.

FIG. 22. Subset of HRS cells. Heatmap summarizing the median expression of selected protein markers on HRS cells clustered using Phenograph. Prior to clustering protein expression values were Arcsinh transformed with a cofactor of 5, clipped at the 99th percentile, and scaled from 0 to 1. The dendrogram represents hierarchical clustering of the heatmap rows (HRS subset clusters) based on Euclidean distance.

FIG. 23. PD1+CD4+ T cells. (Top) Heatmap showing mean expression of inhibitory receptors for PD1+CD4+ T cell subset. (Bottom) Dot plot showing correlation of spatial scores of PD1+CD4+ T cells and selected immune cell markers by imaging mass cytometry (IMC). Dot size and color summarize Pearson correlation values, with positive correlations represented in red and negative correlations represented in blue. Asterisks represent associated p-values (*P<0.05; **P<0.01; ***P<0.001).

FIG. 24. RHL4S vs. Reported Prognostic Markers Forest Plot. The RHL4S risk-class is compared to other reported prognostic markers (y-axis) of (Left) post-ASCT failure-free survival (FFS) and (Right) post-ASCT overall survival (OS) using pairwise Cox regression of two variables. Each dot represents the hazard ratio (x-axis) with the bar representing the 95% confidence interval. Each facet on the y-axis is a different pairwise multivariate Cox regression.

FIGS. 25A-25B. RHL4S on diagnostic biopsy. Kaplan-Meier curves of the high-versus low-risk groups for (25A) post-ASCT failure-free survival (FFS) and (25B) post-ASCT overall survival (OS) as identified by RHL4S from diagnostic biopsy. P values were calculated using a log rank test.

FIG. 26. RHL4S vs. Reported Prognostic Markers Forest Plot in validation cohort. The RHL4S risk-class is compared to other reported prognostic markers (y-axis) of post-ASCT outcomes using pairwise Cox regression of two variables. Each dot represents the hazard ratio (x-axis) with the bar representing the 95% confidence interval. Each facet on the y-axis is a different pairwise multivariate Cox regression.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “biological sample” as used herein denotes a sample taken or isolated from a biological organism (e.g., a subject). In some embodiments, the sample is a biological sample. In some embodiments, the sample or biological sample is a tissue sample. In some embodiments, the sample is a biopsy sample containing tumor tissue from a cancer patient. Non-limiting examples of tissue samples include Hodgkin's lymphoma tissue, ovarian cancer tissue, diffuse large B cell lymphoma (DLBCL) tissue, breast cancer tissue, prostate cancer tissue, melanoma tissue, and combinations thereof.

“Classical Hodgkin lymphoma” is one of the main subtypes of Hodgkin lymphoma. Another main subtype is nodular lymphocyte-predominant Hodgkin lymphoma (NLPHL). Classical Hodgkin lymphoma is characterized by the presence of both Hodgkin and Reed-Sternberg cells. Nodular lymphocyte-predominant Hodgkin lymphoma is characterized by the presence of lymphocyte-predominant cells, sometimes termed “popcorn cells,” which are a variant of Reed-Sternberg cells.

The terms, “patient”, “individual” and “subject” are used interchangeably herein. In an embodiment, the subject is mammal. The mammal can be a human, non-human primate, mouse, rat, dog, cat, horse, or cow, but are not limited to these examples. In some embodiments, the subject is a human.

The terms “marker” or “biomarker” are used interchangeably herein, and in the context of the present invention includes but is not limited to one or more proteins (including but not limited to hormones, antibodies, enzymes, soluble proteins, cell surface proteins, secretory proteins), gene products, protein fragments, peptides, nucleic acids (including but not limited to DNA, RNA, microRNA, siRNA, shRNA), or lipids.

The term “cell” or “cells” or “cell type” as used herein is not limited to a particular type of cell or cells.

In some embodiments, cells and/or markers are labeled. Non-limiting examples of labels include antibody label, isotope label, fluorescent label, fluorochrome label, a fluorophore label, and combinations thereof. Exemplary isotope labels include metal isotopes, such as 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Gd, 162Dy, 163Dy, 164Dy, 166Er, 167Er, 168Er, 169Tm, 170Er, 172Yb, 173Yb, 174Yb, 175Lu, 176Yb and combinations thereof.

“Statistically significant” generally means that the difference between two values has a p-value of ≤0.05, i.e., has a 95% or higher chance of representing a meaningful difference between the two values. In some embodiments, a statistical significant difference has a p-value of ≤0.01, i.e., has a 99% or higher chance of representing a meaningful difference between the two values.

“Processor” refers to a hardware that runs the computer program code. In some embodiments, the term “processor” is synonymous with terms like “controller,” “computer,” and should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

For clustering tools, “k-means” refers to a partitioning method (or algorithm) that divides the dataset into k clusters, each represented by the centroid of the data points in the cluster; “phonograph” refers to a method that clusters cells by constructing a k-nearest neighbor graph and then detecting communities within this graph; and “leiden” refers to an algorithm that refines the cluster partitioning by optimizing a modularity score, leading to the detection of highly connected communities. In some embodiment, k-means is used for identifying spherical clusters in the feature space. In some embodiments, phonograph is used for identifying clusters with varying densities and sizes. In some embodiments, leiden is used for uncovering fine-grained and highly cohesive clusters. For example, Scimap, a python toolkit accessible online, may be used for analyzing spatial molecular data, such as spatial datasets mapped to XY coordinates, and it includes preprocessing, phenotyping, visualization, clustering, spatial analysis and differential spatial testing. As another example, spatstat, an R package accessible online, may be used for analyzing spatial statistics, especially spatial point patterns such as in 2D.

Described herein our innovation uses spatial metrics based on nearest neighbor analysis. Herein we introduce the concept of an interaction radius to computationally isolate local interactions and prevent the data from being influenced by cells that are very far away. As such, we can quickly and consistently process spatial analysis of cancer tissue in an automated fashion to focus on tumor-immune cell biology, which occurs at a localized level. Our method identifies proteins such as CXCR5 that correlate to survival in specific spatial contexts, and we can describe spatial reorganization from diagnosis to the relapsed tumor as it relates to survival and relevant clinical factors such as MHC expression and EBV infection. Thus, in various aspects, our strategy provides a direct, automated algorithm to process spatial data in a highly localized fashion, thus narrowing the scope of spatial analysis to the most relevant regions. With a nearest neighbor strategy, we retain the distance measurements as data tables allowing us to perform other mathematical operations, which lead up to biomarker development, such as clinical laboratory improvement amendments (CLIA) biomarkers which may be used to predict patient outcomes based on initial data. In various embodiments, the product (such as computer program product, or non-transient computer readable medium) provided herein are tailored to customers performing diagnostics using tissue data.

Previous methods disclosed in U.S. Patent Application Publication No. US2022/0336058 calculated a centroid location by calculating a central average point between a set of cells, where the distance from this centroid location was calculated to a target cell.

Herein, the methods differ from those disclosed in US2022/0336058 in that it does not calculate a centroid location but instead draws a fixed distance around a target cell (a circle) and then calculates the distance of the target cell to other cells within the fixed distance. The distance between the cells is divided by the fixed distance of a cell, i.e., the circle radius, and that value is subtracted from 1 to calculate/obtain a “spatial score.” The methods disclosed herein allow for clear identification of cell types and trends, facilitating a prognosis and identification of desirable treatment option based on analysis of patient biopsies. The methods disclosed herein are generalizable and would be especially useful for sparse tumor distribution (such as Hodgkin's Lymphoma) or any cancer lacking a clearly defined cancer boundary.

In various embodiments, a method of performing analysis on a tissue sample, comprising: obtaining an image of the tissue sample, measuring based on the image a distance from a cell of interest of a selected cell type to each of its top nearest cells within a predetermined radius, and obtaining an enrichment score for the selected cell type by calculating an inverse of the distance relative to the predetermined radius. In some embodiments, top nearest cells are a quantity of 3, 4, 5, 6, 7, 8, 9, or 10, or more nearest cells. In some embodiments, top nearest cells are a quantity of 5 nearest cells to the cell of interest. In some embodiments, an inverse of the distance relative to the predetermined radius is to subtract a normalized/scaled distance (normalized/scaled by being divided by the predetermined radius) from 1. In some embodiments, the method includes repeating the measuring step for each cell of the selected cell type. In further embodiments, an enrichment score is an average inverse of the relative distance of all cells of the selected cells types in the image.

In some embodiments, a spatial metric computation is performed by the following steps:
1. Input: Data matrix with cell type labels and x/y coordinates for each ROI (Region of Interest)
   a. Create 5 (or 3, 4, 5, 6, 7, 8, 9, 10 or more) extra data points for each ROI of cells that are positive for every cell type label that are very far away from the image cells.
2. Iterate the next steps over every comparison type label (CD4, CD8, Macrophage, etc).
   a. Measure the distance of each cell to its 5 (or 3, 4, 5, 6, 7, 8, 9, 10 or more) nearest neighbors of comparison cell type.
      i. If there are fewer than 5 (or the 3, 4, 5, 6, 7, 8, 9, 10 or more of this step), the extra data points will satisfy this condition.
   b. Truncate distances to 50 microns.
   c. Calculate the average truncated distance. This value ranges from 0 to 50.
   d. Normalize the average truncated distance by dividing by 50.
   e. Subtract from 1.
   f. This value is the spatial metric (also called “spatial score”).
      i. A separate value exists for each cell type, CD4 spatial, CD8 spatial, etc.
      ii A value of 0 indicates no spatial proximity, 1 indicates maximum overlap.
3. Perform mathematical operations using the spatial metric.
   a. Select cells based on spatial metric cutoffs, e.g., select tumor-proximal cells by choosing cells with tumor spatial metric <0.5.
   b. Cluster cells using spatial metric.
      i. Use data matrix with spatial metric columns.
      ii. Perform phenograph, k-means clustering, etc.
      iii. Define clusters by spatial metric values, e.g., B spatial metric enriched cluster, CD4 and B spatial metric enriched, etc.
   c. Measure sample level features of spatial metric and use for biomarker analysis.
      i. Example, calculate % of cells with spatial metric >a certain value, stratify patients, perform survival analysis.

In some embodiments, the spatial metric computation compares a new sample relative to spatial metric(s) of known sample(s). In some embodiments, two samples or two groups of samples with known treatment outcomes (e.g., survival outcomes) have their spatial metrics computed according to the steps 1, 2, and 3a-3b. In some embodiments, samples with known mutually exclusive (e.g., opposite) treatment outcomes have different spatial metrics, setting two ends of a range/spectrum. In some embodiments, a new sample's spatial metric being similar or closer to one end of the range/spectrum indicates the new sample has a prognosis of likely having a treatment outcome similar to that of the known sample corresponding to the one end of the range/spectrum.

In various embodiments, the distance of each cell to its 5 (or 3, 4, 5, 6, 7, 8, 9, 10 or more) nearest neighbors of every cell phenotype label was calculated, censored at 50 μm, scaled from 0-1, and inverted. These distance metrics describe the local enrichment for a specific cell type to each cell, and were used in clustering using k-means clustering (k=15 or 35, or any numbers in between) to define spatial niches/local microenvironments. Local rosette-dependent protein expression was obtained by selecting rosetting cell types (CD4 T, CD8 T, Treg, Macrophage, B cell), and identifying all cells of the rosetting cell type within the rosetting radius (15 μm) of every cell. The number of rosetting cells within the radius and the mean protein expression were recorded. Ligand receptor expression was defined as the product of the ligand and receptor on the central cell and rosetting cells. Rosette-specific protein expression was calculated by constructing a generalized linear model of rosette size as a function of rosetting cell protein, HRS cell protein, or ligand-receptor expression, and extracting p values for each of the terms.

In various embodiments, methods are provided for calculating an enrichment score for a target cell in a tissue sample, the enrichment score representing enrichment of a selected cell type around the target cell. In some embodiments, the methods are computer-implemented methods. In various embodiments, the methods include the steps of: measuring a normalized distance from the target cell to each of its top nearest cells of the selected cell type within a predetermined radius in the tissue sample or an image of the tissue sample; and performing an inverse operation to determine an inverse of the normalized distance from the target cell to the top nearest cells of the selected cell type. In various aspects, an inverse of the normalized distance is a score representing enrichment of the selected cell type around the target cell.

In some embodiments, the methods further include repeating the steps to calculate an enrichment score for each of a quantity of cells of a same type as the target cell in the tissue sample or the image of the tissue sample.

In some embodiments, the methods further include repeating the steps with a plurality of different selected cell types, so as to calculate respective enrichment score representing enrichment of each of the different selected cell types around the target cell.

In some embodiments, the target cell is of a same type as the selected cell type, so that the enrichment score representing enrichment of the same type of cells as the target cell around the target cell.

In some embodiments, the methods further include repeating the steps to calculate an enrichment score for each of a plurality of target cells of different cell types.

In some embodiments, the target cell is a tumor cell, and the selected cell type is not a tumor (thereby also referred to as a rosetting cell). In some embodiments, the enrichment score represents enrichment of rosetting cells around the target tumor cell, and the rosetting cells are of a selected cell type from an immune cell comprising CD4+ T cell, CD8+ T cell, regulatory T cell (Treg), macrophage, or B cell, or a fibroblast, or an epithelial cell.

In some embodiments, the methods further include repeating the steps to calculate an enrichment score for each of a plurality of target cells with different rosetting cell types. In some embodiments, the number of nearest cells with a distance equal to or less than 15 μm from a central cell in question (i.e., a target cell) is measured. In some embodiments, the nearest cells is of a specific type (e.g., CD4+ cells, or CD8+ cells, or Treg, or macrophages, or B cells). In some embodiments, the number of cells of each specific type is measured. In some embodiments, the protein expression on the central cell as a function of rosetting cells is measured. In some embodiments, multiple protein expression as a function of rosetting cells is measured. In some embodiments, the contribution (or correlation) of multiple protein expression to rosetting cells is measured, for example, using a generalized linear model. In some embodiments, the average protein expression on rosetting cells is used instead of the protein expression on the central cell. In some embodiments, the product (mathematical product of multiplication) of protein expression on the central cell and average protein expression on rosetting cells is determined, and optionally used instead of the protein expression on the central cell in determining contribution to (or correlation with) for example total number of a rosetting cell type.

In some embodiments, the methods further include applying a clustering model, e.g., a K-means clustering model, using data records including the enrichment score for each of the target cells of different cell types or different rosetting cell types to generate apply-output data defining niches of the different cell types or different rosetting cell types in the tissue sample or the image of the tissue sample.

Any clustering models in machine learning are generally suitable for the spatial metric analysis methods disclosed herein, which include but are not limited to K-means clustering, phenograph clustering. Generally K-means clustering model includes an algorithm that performs the following: 1. k initial “means” (in this case k=3, 4, 5, 6, 7, 8, 9, 10, 11, 12, 13, 14, 15, 16, 17, 18, 19, 20, or more) are randomly generated within the data domain; 2. k clusters are created by associating every observation with the nearest mean. For example, the partitions separating the clusters represent the Voronoi diagram generated by the means. 3. The centroid of each of the k clusters becomes the new mean. 4. Steps 2 and 3 are repeated until convergence has been reached.

In some embodiments, the data records exclude enrichment score greater than a selected cutoff value. For example, a cutoff value is selected based on half of the predetermined radius, or 25% of the predetermined radius, or 75% of the predetermined radius. In some embodiments, the inverse operation is subtracting the normalized distance from 1, and the cutoff value may be 0.5, 0.75, or 0.25. In some embodiments, the data records exclude enrichment score lower than a selected cutoff value. In further embodiments, the data records exclude enrichment score higher than a first selected cutoff value and lower than a second selected cutoff value (the first selected cutoff value being greater than the second selected cutoff value).

In some embodiments, the top nearest cells are 2, 3, 4, 5, 6, 7, 8, or more cells of the selected cell type.

In some embodiments, the top nearest cells are 5 cells of the selected cell type.

In some embodiments, the normalized distance is a distance divided by the predetermined radius.

In some embodiments, the distance is between a center or centroid of the target cell to a center or centroid of each of the top nearest cells.

In some embodiments, the distance is between an edge of the target cell to an edge of each of the top nearest cells, optionally the distance being a shortest point on respective top nearest cell to an edge of the target cell.

In some embodiments, the methods further include determining an average normalized distance from the target cell to the top nearest cells, wherein performing the inverse operation is performing the inverse operation on the average normalized distance, so as to determine the inverse of the average normalized distance from the target cell to the top nearest cells. In further embodiments, the determining of the average normalized distance is determining an arithmetic mean of the normalized distance from the target cell to each of the top nearest cells.

In some embodiments, an inverse of the normalized distance from the target cell to each of the top nearest cells are determined, and the method further determines an average inverse of the normalized distance from the target cell to the top nearest cells. In further embodiments, the determining of the average inverse of the normalized distance is determining an arithmetic mean of the inverse of the normalized distance from the target cell to each of the top nearest cells.

In some embodiments, the inverse operation is subtracting the normalized distance from a fixed number, optionally the fixed number being 1 or 100%. In other embodiments, the inverse operation is dividing a fixed number by the normalized distance. In additional embodiments, the inverse operation is calculating a multiplicative inverse of the normalized distance.

In some embodiments, the predetermined radius is about 50 μm, about 60 μm, about 70 μm, about 80 μm, about 90 μm, or about 100 μm.

In some embodiments, the measurement is performed with a mass cytometry image of the tissue sample. In some embodiments, the measurement is performed on a fluorescence image, such as multicolor immunofluorescence image. In some embodiments, the image is obtained for a snap-frozen biopsy sample or a formalin-fixed and paraffin-embedded (FFPE) biopsy sample from a subject.

In some embodiments, the tissue sample includes a tissue from a subject with a cancer, having been treated against a cancer, or having a refractory or relapsed cancer.

In some embodiments, the cancer is Hodgkin's lymphoma (e.g., classical Hodgkin lymphoma), ovarian cancer, diffuse large B cell lymphoma (DLBCL), or another non-Hodgkin lymphoma.

In some embodiments, the tissue sample has been treated with a panel of labeled antibodies against at least 10, 20, 30, 40, 50, or more marker proteins in the tissue sample, so that the target cell and/or the selected cell type is defined based on presence of one or more selected marker proteins.

In some embodiments, the methods include measuring label intensities of the one or more of the at least 10, 20, 30, 40, 50, or more marker proteins in the tissue sample or the image of the tissue sample.

Additional embodiments provide a system for performing spatial metric analysis, and the system includes or is a combination of: a processor operable to execute computer executable instructions; a memory operable to store computer executable instructions executable by the processor; and computer executable instructions stored in the memory and executable to perform the steps of enrichment score calculation and/or spatial metric analysis disclosed herein.

Additional embodiments provide a computer program product for performing spatial metric analysis, and the product includes: a computer readable medium; and computer program instructions, recorded on the computer readable medium, executable by a processor, for performing the steps of enrichment score calculation and/or spatial metric analysis disclosed herein. Further embodiments provide a non-transient computer readable medium, which includes computer executable instructions, recorded on the non-transient computer readable medium, executable by a processor, for performing the spatial metric analysis disclosed herein or any steps related thereto.

Further embodiments provide methods of stratifying, assessing, or monitoring a patient with ovarian cancer. In various embodiments, enrichment scores for all cells are measured/determined in a region of interest in a sample or an image of the sample; cells with an enrichment score outside a predetermined range (e.g., ‘outside’ being higher than a predetermined ‘high’ cutoff value and lower than another predetermined ‘low’ cutoff value) are removed or excluded from subsequent step(s); and a patient is stratified, or a pool of patients are compared, based on one or more selected marker proteins' expression levels in the cells with an enrichment score within the predetermined range.

In some embodiments, the methods include detecting a higher enrichment score according for stromal cells enriched with a same type of stromal cells nearby, optionally immune cells and/or fibroblasts (optionally podoplanin-positive fibroblasts), in a tissue sample obtained from the patient or an image of the tissue sample, relative to that of a control subject who has relapsed after 15 months following debulking surgery for ovarian cancer, thereby indicating that the patient is likely to have relapse of the ovarian cancer within 15 months following a debulking surgery for the ovarian cancer.

In some embodiments, the methods further include or alternatively include detecting a higher percentage of B cells in a region with the higher enrichment score for the fibroblasts in a tissue sample obtained at primary tumor stage from the patient, relative to that in a tissue sample obtained at tumor recurrence stage from the patient, thereby indicating that the patient is likely to have relapse of the ovarian cancer within 15 months following a debulking surgery for the ovarian cancer.

In additional embodiments, methods for treating a subject indicated likely to have early relapse of ovarian cancer (e.g., following debulking surgery) are provided, which include administering additional therapy (e.g., chemotherapy or platinum-based chemotherapy) against the ovarian cancer to the patient indicated likely to have relapse within the 15 months.

Further embodiments provide methods of stratifying, assessing, or monitoring a patient with Hodgkin's lymphoma (HL).

In some embodiments, the methods include detecting a lower enrichment score for tumor cells enriched with CD8+ T and/or B rosetting cells, and/or detecting a higher CXCR5 expression level in the tumor cells and/or the rosetting cells, in a tissue sample obtained from the patient or an image of the tissue sample, relative to that of a control subject who has relapsed later than 1 year or has no relapse, thereby indicating that the patient is likely to have poor outcome or early relapse within 1 year from initial treatment against the Hodgkin's lymphoma. In some embodiments, the methods include detecting a higher enrichment score for tumor cells enriched with CD8+ T and/or B rosetting cells in a tissue sample obtained from the patient or an image of the tissue sample, relative to that of a control subject who has relapsed within 1 year, thereby indicating that the patient is likely to have good outcome, no relapse, or late relapse after 1 year from initial treatment against the Hodgkin's lymphoma.

In some embodiments, the methods further include or alternatively include detecting a co-expression pattern of marker proteins in the target tumor cell and in rosetting cells as follows:
(i) inducible costimulatory ligand (ICOSL) in the target tumor cell and inducible T cell co-stimulator (ICOS) in rosetting macrophages,
(ii) galectin-9 in the target tumor cell and T cell immunoglobulin and mucin domain-containing protein 3 (TIM3) in rosetting B cells and/or rosetting macrophages, and/or
(iii) galectin-9 in the target tumor cell and V-domain Ig suppressor of T cell activation (VISTA) in rosetting CD4+ T cells, rosetting CD8+ T cells, rosetting B cells, and/or rosetting macrophages,

thereby indicating that the patient is likely to have poor outcome or early relapse within 1 year from initial treatment against the Hodgkin's lymphoma.

In some embodiments, the methods include detecting a higher CXCR5 expression level in the tumor cells and/or the rosetting cells in a tissue sample obtained from the patient or an image of the tissue sample, relative to that of a control subject who has relapsed later than 1 year or has no relapse, thereby indicating that the patient is likely to have poor outcome or early relapse within 1 year from initial treatment against the Hodgkin's lymphoma.

In additional embodiments, methods are provided for treating a subject indicated likely to have poor outcome or early relapse within 1 year from initial treatment of Hodgkin's lymphoma, and the methods include administering additional therapy (e.g., chemotherapy) to the patient indicated likely to have poor outcome or early relapse within the 1 year.

Some embodiments provide methods for treating refractory or relapsed classical Hodgkin lymphoma (cHL) in a human subject, the methods include: providing a salvage therapy comprising autologous stem cell transplantation (ASCT) or a combination of high-dose chemotherapy and the ASCT to the human subject if the human subject is detected in a biopsy sample of the human subject with presence of enrichment of CXCR5+ B cells around a Hodgkin and Reed Sternberg (HRS) tumor cell and with absence of CXCR5+ HRS tumor cells and absence of enrichment of CXCL13+ macrophages or PD-1+CD4+ T cells around the CXCR5+ HRS tumor cells.

In some embodiments, a method for treating refractory or relapsed classical Hodgkin lymphoma (cHL) in a human subject includes: providing allogeneic bone marrow transplantation, a CD30 targeting treatment, and/or brentuximab vedotin, or a new therapy under clinical trial, to the human subject if the human subject is detected in the biopsy sample with presence of the CXCR5+ HRS tumor cells and enrichment of the CXCL13+ macrophages and/or PD-1+CD4+ T cells around the CXCR5+ HRS tumor cells.

In various aspects, the enrichment of CXCR5+ B cells refers to the presence of a quantity (2 or more) of CXCR5+ B cells within a radius of no more than about 50 μm (or less than 100 μm, 90 μm, 80 μm, 70 μm, 60 μm, 50 μm, 40 μm, 30 μm, 20 μm, 15 μm or 10 μm) from the HRS tumor cell. In various aspects, enrichment of CXCL13+ macrophages and/or PD-1+CD4+ T cells refers to the presence of a quantity of CXCL13+ macrophages and/or a quantity of PD-1+CD4+ T cells, respectively, within the radius from the CXCR5+ HRS tumor cells.

In various aspects, a high-dose chemotherapy comprises a higher dose of chemotherapy than that of a prior/initial chemotherapy to which the cHL is refractory or has relapsed.

Additional embodiments provide methods for providing prognosis of salvage therapy to a human subject having refractory or relapsed classical Hodgkin lymphoma (cHL), and treating the human subject, based on enrichment scores (also referred to as spatial interaction scores). In various implementations, we establish t-values to obtain weights that we use to calculate linear predictor score(s) (LPS), we take our stable high and low risk groups and calculate LPS for all patients in those groups to obtain the mean and standard deviation needed to create phi distributions, then we simply plug in each new patient's LPS score into the formula. LPS score is calculated per patient, using an equation:

$$LPS ( X ) = {\sum }_{j} {a}_{j} {X}_{j} ,$$

wherein Xⱼ is a standardized spatial score per patient, which can be obtained any of several ways, such as the average spatial score of each cell, the median, etc.; and aⱼ is defined by the t-statistic of the single variable generalized linear model for the variable in question. The t statistic may be derived from a simple model of relapse˜variable. We calculate the probability of a sample/patient falling under the high or low risk group of relapse (or any binary outcome) with that formula. The two types of phi distributions are the normal density functions for the two groups that we are trying to assign probabilities for. So if we are trying to decide if a new sample is high risk or low risk, we have the mean and sd for those groups, those are used to generate the phi distributions. The distribution of LPS scores is the collection of actual LPS scores calculated per patient/sample. There is an assumption that they are normally distributed (a bell shaped curve), so we approximate it with the phi normal density function using the mean and sd. Essentially we are plotting the two bell curves and then for any new patient sample, we calculate the LPS score, and we see how well the two bell curves overlap at that score. LPS(X) is the output of the model, and we compare each patient's output to the expected output of the two risk populations and calculate the probability based on the ratio of expected model outputs for the two risk groups that give the patient's output. The LPS is a standardized score per patient, so the single cell calculations all happen under the hood, and LPS summarizes where all cells in a patient generally fall. The LPS itself is not a distribution, rather it is a linear weighted combination of standardized spatial measurements. LPS distributions would be in the context of all patients as well as risk groups.

In a normal distribution, u₁/sigma₁ and u₂/sigma₂ are two risk groups' means and standard deviations used to generate phi distributions. LPS(X) is another formula that is applied to X, a new patient sample, to generate a model score, which is then entered into the 3 phi distributions, shown as

$$P ( X ) = \frac{ϕ ( LPS ( X ) ; {μ}_{1} , {σ}_{1}^{2} )}{ϕ ( LPS ( X ) ; {μ}_{1} , {σ}_{1}^{2} ) + ϕ ( LPS ( X ) ; {μ}_{2} , {σ}_{2}^{2} )} ,$$

which generates a value between 0 and 1 which is the probability that the patient is in a particular risk group.

Each spatial score (or “enrichment score”) is typically within the range of 0-1; whereas LPS(X) does not necessarily follow the same range as it is a linear combination of individual standardized scores.

Further embodiments provide treatment methods. In some embodiments, a patient having lymphoma (e.g., classical Hodgkin lymphoma) and prognosed with a low likelihood of relapse (if a chemotherapy or a salvage therapy is given) will be given the chemotherapy (e.g., if the patient has not had prior Hodgkin lymphoma treatment) or the salvage therapy (e.g., if the patient has relapsed from or refractory to a previous “initial” treatment, or if the patient has treatment-resistant classical Hodgkin lymphoma). In some embodiments, salvage therapy includes autologous stem cell transplantation (ASCT) or a combination of high-dose chemotherapy and the ASCT.

In some embodiments, a patient having lymphoma (e.g., classical Hodgkin lymphoma) and prognosed with a high likelihood of relapse or high likelihood of early relapse (e.g., within 10 11, 12, 13, 14, or 15 months if a chemotherapy or a salvage therapy is given) will be given a different treatment, such as allogeneic bone marrow transplantation, a CD30 targeting treatment, and/or brentuximab vedotin.

In some embodiments, a patient having ovarian cancer and prognosed with a high likelihood of relapse or early relapse (e.g., within 12, 13, 14, 15, 16, 17, or 18 months following a debulking surgery) will be given a therapy such as chemotherapy, or immunocheckpoint inhibitors, either in replace of or in addition to the debulking surgery.

In some embodiments, a patient having ovarian cancer and prognosed with a low likelihood of relapse will be given a debulking surgery.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Changes in the Ovarian Cancer Microenvironment Associated with Tumor Progression and Chemoresistance

Imaging mass cytometry (IMC) is revealing new insights on tumor architecture, showing that the infiltration and interaction of immune cells, tumor cells, and stromal cells informs the functional activity of those cells and the whole tumor. Imaging analysis has long been used to study cancer and predict patient outcomes via H&E pathology and immunohistochemical (IHC) staining, and IMC is bringing a high-multiplexity revolution to that spatial analysis. Metastatic Ovarian Cancer responds poorly to standard platinum-based chemotherapy for yet unknown reasons. Studying stroma, tumor, and immune subsets such as T cells, B cells, and macrophages alone with standard imaging methods like H&E has revealed limited insights, but without highly multiplexed imaging a unified picture of how these cells interact has not emerged. Here we used IMC to analyze a cohort of 42 patients with paired primary tumor, concurrent tumor, and recurrent tumor after chemotherapy, where the recurrent tumor emerged between 1 month and 5 years after treatment. While recurrence is extremely common after treatment, we sought to determine functional protein and spatial factors predictive of delayed recurrence and positive outcomes. We analyzed 260 patient samples in total and over 1.5 million cells using cell segmentation and single cell analysis pipelines to identify tumor cells and major immune and stromal cell types and performed single cell spatial analysis to explore cellular interactions between these cells. First, we analyzed the same tissue using IHC and a machine learning classifier for H&E, showing that IMC reproduced the established imaging findings. Next, we used the multiplexing capability of IMC and found statistically significant differences in spatial architecture by early recurrence, with more stroma-tumor interaction and tumor clustering predicting poor outcomes. In contrast, immune infiltration and the formation of tertiary lymphoid structures was associated with delayed recurrence. This is the largest highly multiplexed ovarian cancer study to date, describing the phenotypes and structure of ovarian cancer in deep detail. We were able to match the architecture to tumors collected from the same patients over time, to show how spatial protein features were associated with early and late recurrence of the tumor.

### Background:

Ovarian cancer is a leading cause of cancer death in women. It is difficult to diagnose, leading to poor survival due to late detection. Tumor tissue composition is linked to outcomes, wherein tumor, stroma, and immune cells are heterogeneously observed and subtype classification of heterogeneity shows survival differences, according to Tothill et al., Clinical Cancer Research, Volume: 14, Issue: 16, Pages: 5198-5208, 2008. Previous measuring the tumor microenvironment was imprecise, wherein ovarian cancer was often profiled qualitatively or semi-quantitatively. Wang et al. in Cell, Volume 165, Issue 5, 19 May 2016, Pages 1092-1105, 2016 indicated spatial interactions are associated with clinical outcome, wherein fibroblasts and stromal CD8 T cells influence chemoresistance, thereby survival; specifically, high stromal fibroblasts and low stromal CD8 T cells are associated with low survival.

### Techniques

Imaging mass cytometry resolves major cell types, for example, aSMA, PanKeratin, and CD8 may be resolved in one image; and imaging mass cytometry provides >30-plex analysis. Tumor microarrays are employed.

### Hypothesis:

1) Tumor region selection is biased. 2) Tumor biology is relatively unchanged within a patient. 3) Patient comparisons between similar regions are more relevant and less biased.

### Our Methods and Observations

Cell-type specific spatial enrichments describe the tumor microenvironment (TME). Significant associations revealed within digital biopsies. T-reg within-patient trends are associated with relapse.

### Conclusions:

42 patients with matched samples were studied over tumor progression. IMC single cell analysis revealed few significant differences at the sample level. Spatial organization was highly heterogeneous. After digitally isolating specific sample regions, significant observations emerged. Additionally we have identified CD11b+ tumors; tertiary lymphoid structure (TLS) detection; and biomarker discovery pipeline with hybrid protein/spatial analysis.

### Example 2. Shifting Challenges in Single Cell Spatial Analysis for Solid and Hematological Tumors

### Background

Single cell biology is at a crossroads, with multi-omics and spatial emerging as two major branches, addressing intercellular and intracellular biology, respectively. Spatial biology is important to cancer immunotherapy to understand the immune-tumor cell-cell interface.

Imaging mass cytometry (IMC) is one of many next-generation technologies used to perform highly multiplexed spatial protein analysis. IMC is compatible with archival formalin-fixed, paraffin-embedded (FFPE) tissue and has well-developed panels for immuno-oncology. Along with spatial transcriptomics, these tools profile immune-stroma-tumor interactions, local cellular superstructure, and cell signaling.

We rely on our large patient tissue resources with clinical data readily available and supporting data (genetics and clinical) to assay ovarian cancer, colorectal cancer (CRC), and aggressive B cell lymphoma.

Solid vs. Hematological Cancer Analysis:

We analyzed four different aspects: cell density differential, marker decisions, existing microstructure, and extracellular biology; and discovered that hematological malignancies are dense, which was computationally challenging to resolve single cells. Tissue microstructure has length scales, which focus on local microenvironments and wherein cell interactions beyond a distance threshold can be ignored. Immune tumors shared biology allows deep tumor/immune profiling.

Tissue should be compared like-for-like; depending on the size scale of tissue microstructure, an IMC sized sample represents different aspects of the tumor structures (TLS).

Tissue-specific expression: each row represents a distinct spatial microenvironment and the protein expression of specific cells in that environment.

### Example 3. Matchmaking and Single Cell Spatial Biology for Combination Therapy

### Background:

Imaging mass cytometry (IMC) is used to study spatial protein expression, single cell heterogeneity and spatial interactions among immune cells in cancer. IMC is advantageous for single cell spatial biology in that it is convenient ˜40-plexing, FFPE tissue compatible, and supports dynamic range and stability. It combines techniques of metal ion-conjugated antibody staining, antibody panel, and tissue microarray.

Techniques for analyzing spatial biology is rapidly growing:

| | | Single Cell | Protein | | | | Time for | |
|---|---|---|---|---|---|---|---|---|
| Method | Vendor | (resolution) | RNA | Equipment | Cost | Parameters | imaging | Comments |
| Serial | homebrew | Yes | Protein or | Requires | $ | up to 10 | minimal, scan | software |
| IHC | | (<1 um) | RNA | scanner | | | after each | multiplex |
| | | | | | | | round | |
| Multiplex | Akoya, | Yes | Protein | Stainer and | $$-$$$ | 6 (Akoya) | scan once, | |
| IF | Ultivue, | (<1 um) | | imager | | 21 | spectral | |
| | RareCyte | | | | | (RareCyte) | deconvolution | |
| GeoMX | Nanostring | No | Protein OR | Scanner | $$$ | 1000+ | 16 slides a | No image |
| | | (10 um) | RNA | and | | genes | day | reconstruction |
| | | | | NCounter | | Whole | | |
| | | | | | | trans. | | |
| Visium | 10x | No | RNA | Oligo | $$$ | Whole | Time for | |
| | | (100 um) | (protein) | spotted | | trans. | sequencing | |
| | | | | slides | | | | |
| CODEX | Akoya | Yes | Protein | Fluidics, | $$$ | 35+ | 3-6 hours/ | Semi-custom |
| | | (0.3 um) | | microscope | | | slide | panel |
| CosMx | Nanostring | Yes | RNA/protein | Box | | 1000/100 | 8 slides/week? | Early access |
| MERFISH | Vizagen | (<1 um) | RNA | Box | | 500 | | only |
| | | Yes | | | | | | No FFPE |
| | | (<1 um) | | | | | | |
| Mass | IonPath | Yes(0.35-1 | Protein | specialized | $$ | 42 | 35 min for | Flexible |
| Cytometry | Fluidigm | um) | | imaging | | | 0.8 mm² ~90 | panel design |
| | (Hyperion) | Yes (1 um) | | equipment | | | min for | |
| | | | | | | | 1 mm² | |

Spatial Biology as IF/IHC supports ˜40-plex imaging and large dynamic range.

Spatial Biology as Flow Cytometry: looks at basic cell types and proportions, supervised or unsupervised, and gating vs clustering.

Spatial Biology as scRNAseq: can be used for tumor microenvironment (TME) analysis to discover new subtypes and classification.

Spatial Biology as H&E/IHC: Quantify spatial interactions between cells; Identify cellular organization; and Niches/Neighborhoods. Spatial Heterogeneity: Tissue size scales, local structures (TLS).

Spatial Biology in Cell-Cell Interactions: study cell-cell distance, phenotype-specific interactions, local protein expression, and extracellular measurements.

### Study of Ovarian Cancer Cohort:

We studied the patient population, and discovered ovarian cancer TME heterogeneity. Ovarian cancer spatially guided analysis revealed that small tissue regions are highly sensitive to sampling and that “digital biopsies” allowed for relevant comparisons.

### Diffuse Large B Cell Lymphoma (DLBCL) Preliminary Study

Other projects based on methods disclosed herein include T Cell Migration Chemokine Analysis and Migration Modeling, T-Cell Receptor Repertoire Analysis to Quantify T Cell Responses, and TCR Sequence Analysis to study T Cell Antigen Specificity.

### Example 4. Spatially-Resolved Biomarkers of the Tumor Immune Microenvironment in Relapsed/Refractory Hodgkin's Lymphoma

The tumor microenvironment (TME) is the complex milieu of cells and molecules surrounding the tumor. Immune cells in the TME have been bent to therapeutic purposes with remarkable results, but immunosuppressive signaling from other immune cells, tumor, and stroma limits the potential of cell and immuno-therapies. Single cell methods have been used to great effect to identify subpopulations of cells that are immunosuppressive (or anti-tumor). However, there is limited information on the spatial organization of these subpopulations within the TME, which in turn determines the influence of checkpoint signaling, T cell synapse formation, immune cell infiltration, and other essential parameters for immunotherapy. Here we apply Imaging Mass Cytometry (IMC), a technology to perform ˜40-plex protein analysis with 1 micron resolution in tissue, to study a cohort of 260 matched samples at diagnosis and after relapse from 90 patients with relapsed/refractory Hodgkin's Lymphoma. We analyzed over 7 million cells for their phenotype and spatial organization. Hodgkin's represents a unique spatial TME due to the sparse tumor distribution of Hodgkin's Reed-Sternberg tumor cells. Yet, even though it is difficult to disentangle signaling in Hodgkin's tumor cells-which are immune derived—from the immune TME, Hodgkin's is highly receptive to checkpoint inhibitors among lymphomas and insights gleaned from the Hodgkin's TME could better inform immunotherapies across lymphomas. We use IMC to describe spatial features of the tumor-cell subtypes and their positioning—that correlate to clinical outcomes. We identify proteins such as CXCR5 that correlate to survival in specific spatial contexts, and we describe spatial reorganization from diagnosis to the relapsed tumor as it relates to survival and relevant clinical factors such as MHC expression and EBV infection. We also describe cell resetting, a localized recruitment of immune cells to tumor that is characteristic of Hodgkin's. (T-cell resetting refers to the presence of CD4+ T cells that surround, protect, and promote survival of tumor cells, which is unique to Hodgkin lymphoma.)

### Background:

Immunotherapy has been highly successful in lymphomas, wherein ˜90% response to anti-PD-1 (Hodgkins) and ˜10% response in DLBCL; and FDA approved CAR-T products for lymphoma, myeloma resulted in ˜60% relapse rate (Ansell et al, NEJM, 2015; Neelapu et al, NEJM, 2017). Immune-rich tumor microenvironments (TME) and oncogenic signaling: HL: Rare CD30+ tumor cells, T cell recruitment; Cell subsets associated with prognosis, outcomes: HL: CD68+ macrophages, etc. (Scott and Gascoyne, Nat. Rev. Cancer, 2014; Stiedl et al, NEJM, 2010).

Imaging Mass Cytometry profiles ˜40 proteins at 1 μm resolution, which can be used for protein expression, phenotyping, and spatial arrangement.

### Study of Hodgkin's Lymphoma Cohort and Experimental Design:

Classical biomarker design using cell phenotypes: Patient samples stratified by pathology (CD68+); and advanced pathology (Roemer et al, Cancer Immunol. Res., 2016; Stiedl et al, NEJM, 2010). With highly multiplexed spatial analysis, one may need to prioritize two out of three of: cohort size, sampling areas, and deep profiling.

Effective Comparisons in Highly Multiplexed Spatial Data: Small sampling areas are highly sensitive to spatial heterogeneity; Local arrangement of cells (rosettes) is associated with genetic features. Define microenvironments (rosettes, niches); Compare samples by microenvironment; and Microenvironments may not be present in all samples.

In all, IMC (MIBI/CODEX/IBEX/etc) spatial protein analysis revealed 90 relapsed/refractory Hodgkin's lymphoma patients; Atlas of tumor spatial architecture: 40+ proteins of interest; and a centralized resource for biomarker validation and discovery: Spatial features correlated with survival or patient status.

### Example 5. Spatial Protein Analysis of the Tumor Microenvironment and Biomarkers in Hodgkin's Lymphoma

Introduction: The tumor microenvironment (TME) is the complex milieu of cells and molecules surrounding the tumor. Single cell methods have been used to great effect to identify subpopulations of cells that have pro- or anti-tumor properties, and selectively modulating these has great therapeutic benefits, especially in immunotherapy. However, there is limited information on the spatial organization of these subpopulations, which determines how they signal and their therapeutic potential. Single cell resolved spatial computational analysis is needed to describe the complex interactions of the TME and their effect on patient outcomes. Hodgkin's Lymphoma presents a unique spatial TME due to the sparse tumor distribution of Hodgkin's Reed-Sternberg tumor cells. Yet even though it is difficult to disentangle signaling in Hodgkin's tumor cells-which are immune derived—from the immune TME, Hodgkin's is highly receptive to checkpoint inhibitors among lymphomas and insights gleaned from the Hodgkin's TME could better inform immunotherapies across lymphomas.

Materials and Methods: Here we apply Imaging Mass Cytometry (IMC), a technology to perform ˜40-plex protein analysis with 1 micron resolution in tissue, to study a cohort of 260 matched samples at diagnosis and after relapse from 90 patients with relapsed/refractory Hodgkin's Lymphoma. We developed a computational pipeline to perform cell phenotyping, spatial analysis, and biostatistics, to describe tumor architecture and propose putative biomarkers of Hodgkin's clinical response and relapse. A new feature of the pipeline is to quantify proteins and spatial analysis on the same numerical scale for each cell, to generate hybrid biomarkers.

Results and Discussion: We analyzed over 7 million cells for their phenotype and spatial organization. We use IMC to describe spatial features of the tumor-cell subtypes and their positioning—that correlate to clinical outcomes. We identify proteins such as CXCR5 that correlate to survival in specific spatial contexts, and we describe spatial reorganization from diagnosis to the relapsed tumor as it relates to survival and relevant clinical factors such as MHC expression and EBV infection. A significant conceptual advance was to use spatial metrics to perform “digital biopsies”, a selection of tumor regions comparable across patients. We also describe cell rosetting, a localized recruitment of immune cells to tumor that is characteristic of Hodgkin's. We validated multiple existing biomarkers in the literature using our data set and proposed novel biomarkers using IMC data.

FIG. 40 in the priority U.S. provisional patent application No. 63/470,740 depict IMC analysis of Hodgkin's Lymphoma. A. IMC generates a 40-plex protein image. B, C. The image is converted to single cells and analyzed for protein and spatial features. D. A UMAP of cell phenotypes and the spatial interactions of specific phenotypes. E. The spatial and protein values are used to generate predictive biomarkers of clinical response.

Conclusions: Spatial analysis of the HL microenvironment revealed composite features of the TME that predict clinical outcomes. These features cannot be described using single cell tools or low-plexed imaging, and represent a truer picture of HL biology. The pipeline developed here can be universally applied to other spatial protein data for biomarker discovery and analysis, and the biomarkers proposed here will be validated with IHC.

### Example 6. Spatiotemporal Architecture of High-Grade Serous Ovarian Carcinoma

### Abstract:

High-grade serous ovarian carcinoma (HGSOC), the deadliest form of ovarian cancer, is typically first diagnosed after it has metastasized and almost always relapses after standard-of-care platinum-based chemotherapy. Targeted therapies and immunotherapies are effective in only a small subset of patients, likely due to advanced tumor stage, inherent heterogeneity, and immune suppression and/or tumor-promoting signaling from the tumor microenvironment. There is a large gap in understanding how spatial heterogeneity and intercellular signaling contribute to HGSOC progression and early relapse. We used Imaging Mass Cytometry (IMC) and a HGSOC tissue microarray of patient-matched pre-chemotherapy primary tumors, synchronous metastases, and metachronous post-chemotherapy recurrent metastases from 42 patients to determine the spatiotemporal arrangement of different cell types during HGSOC progression. We found that tumors from patients with early relapse exhibit distinct patterns of immune cells, fibroblasts, and epithelial cells, including malformed tertiary lymphoid structures and increased presence of podoplanin-expressing fibroblasts, across all stages analyzed. Changes in T cell localization between primary and synchronous metastatic tumors were also associated with early relapse, independent of the concentration of total T cells. Our highly multiplexed IMC data was consistent with data obtained by standard histology and immunohistochemistry and also demonstrated the additive value of highly multiplexed analyses.

### Significance:

High-grade serous ovarian cancer is usually detected after it has metastasized to multiple organs in the peritoneal cavity. The success of standard treatments, such as debulking surgery and chemotherapy, is largely dependent on the architecture of the tumor microenvironment, including the proportions of different subtypes of cancer-associated fibroblasts and immune cells. It is still poorly understood how cancer metastases to different organs shape the tumor microenvironment and how the tumor microenvironment changes over time in response to treatment. Here, we used spatial protein analysis to dissect the evolution of the ovarian cancer microenvironment and identify cell types and dynamic spatiotemporal interactions associated with early cancer recurrence.

### Introduction

The most common and most lethal subtype of ovarian cancer is high-grade serous ovarian carcinoma (HGSOC). Standard treatment for HGSOC combines surgical cytoreduction with platinum-based chemotherapy. Typically, this treatment is initially successful in achieving remission, but cancer recurs in the vast majority of cases. Although patients with recurrent disease might respond to additional cycles of chemotherapy, most ultimately develop resistance. Immune cells can promote and/or inhibit tumor progression depending on signals received from the tumor microenvironment. In particular, cancer-associated fibroblasts (CAFs) are emerging as important regulators of immune cell activity and tumor development, mediated by proteins such as fibroblast activation protein (FAP) and podoplanin. Multiple studies have reported an uneven distribution of different immune cell types and/or different maturation stages of the same cell type across different tumors and even within the same tumor. These observations provided the basis for the development of ‘immunoscores’ as predictors of survival, metastasis, and therapeutic response.

In primary HGSOC, longer survival has been associated with tumor-infiltrating CD8+ T cells and plasma cells in tertiary lymphoid structures (TLS). Based on the presence and distribution of CD8+ T cells in ovarian cancer, three main spatial patterns have been described: infiltrated, excluded, and desert. While desert tumors consist primarily of epithelial cells and are largely devoid of immune cells, infiltrated tumors have abundant immune infiltrates evenly distributed in cancer and stromal regions. Excluded tumors typically exhibit a higher CAF content than the infiltrated and desert tumors and the majority of T cells present are not in direct contact with cancer cells. Excluded tumors are associated with poor survival although it is unclear if this is due to dysfunctional T cells sequestered in the tumor stroma or limited access to chemotherapy due to the dense extracellular matrix (ECM) secreted by CAFs. While immune cell distribution and function have been extensively studied in primary tumors, less is known about the distribution of immune cell subsets during tumor progression and chemotherapy resistance. In a mouse model of ovarian cancer it has been shown that cancer progression is primarily driven by a switch from immunoproficient to immunosuppressive immune cell types rather than by a loss of tumor immunogenicity. It is currently unknown how this equilibrium is established and how the ratios and spatial distribution of different stromal cell types affect tumor progression and response to therapy.

Ratios between different cell types in a tumor, including cancer cells, fibroblasts, and immune cells, can be studied in detail with single-cell RNA-seq analyses. Single-cell transcriptomic studies in ovarian cancer have contributed much to our understanding of HGSOC; however, most of the studies were done using samples from a small number of patients. Olalekan et al. analyzed omental metastases from 6 ovarian cancer patients, of which 4 were HGSOC. Izar et al. analyzed single-cell transcriptomes in ascites from 11 HGSOC patients. Although Pietila et al. conducted RNA-seq expression analysis of primary, metastatic, and recurrent ovarian cancer from 32 patients, they focused on genes involved in ECM remodeling. Using RNA-seq analysis, Kreuzinger et al. compared patient-matched primary and recurrent fresh-frozen tissue samples from 66 HGSOC patients and found that the tumor microenvironment was the most significant contributor to the differential gene expression. Using NanoString gene expression profiles and immunohistochemistry (IHC) analyses of formalin-fixed paraffin-embedded (FFPE) samples, Westergaard et al. investigated the molecular features of matched primary and recurrent HGSOC from 9 patients and found that gene signatures of fibroblasts and immune cells were often expressed at higher levels in recurrent tumors. While these studies showed the heterogeneity of HGSOC, they did not focus on the spatial relationships between cell types and tissue architecture.

More recently, spatial resolution has been added to single-cell analysis. Commercially available tools for highly multiplexed spatial protein analysis include Imaging Mass Cytometry (IMC), Multiplexed Ion Beam Imaging (MIBI), and CO-Detection by indEXing (CODEX). When applied to tumor studies, these spatially-resolved methods can provide an added layer of spatial context by describing the microenvironmental niches where each cell type can be found. This is essential for clinical translation, as molecular analysis (e.g. RNA-seq) is relatively costly and imaging remains the most common modality for biomarkers and clinical decision making. Whereas typical biomarkers focus on cellular composition as percentages and qualitative expression levels, quantification of higher-order cell patterns within tissue, found at tissue interfaces, in cell-cell interactions, and other types of heterogeneity, is still in its infancy in pathology. Spatial patterns represent the dynamic biology of tumors and have shown promise as more accurate biomarkers, but spatial analysis has not yet been widely applied in ovarian cancer, and the studies published to date were conducted on small numbers of samples. An IMC analysis of pre- and on-treatment immune therapy biopsy samples from 6 patients showed that treatment response correlated with an increase in CD8+ T cells and FoxP3+ cells. Additionally, an IMC study of primary HGSOC from 20 short-term (overall survival ≤20 months) and 21 long-term (overall survival ≥80 months) patients showed different densities of Granzyme+CD8+ cytotoxic T cells, CD45RO+CD4+ memory T cells, B7+H4+ Keratin+ tumor cells, two subtypes of CD73+ fibroblasts, and a subset of CD31+ endothelial cells in tumors from the two patient groups. A spatially resolved transcriptomic analysis of 12 HGSOC patients with different response to neoadjuvant chemotherapy emphasized the importance of stromal signaling and immune cell localization.

Currently, the costs associated with advanced spatial imaging tools and computational methods, including machine infrastructure, computational power, and bioinformatics expertise, stifle clinical translation. Bridging the gap between developing technologies such as IMC and traditional methods of clinical oncology presents a major challenge in the modern single cell-omics era. The clinical analogues to IMC are immunohistochemistry (IHC), which is limited to a few markers but can be performed quickly and inexpensively, and hemotoxylin/eosin (H&E), which captures a wide range of interpretable histological features. In ovarian cancer, multiplex IHC has been used to correlate patient outcomes and tumor molecular characteristics with the distribution of major immune cell subtypes, including T cells, B cells, and plasma cells. Here we show that IMC not only reproduces equivalent histologic analyses, but also generates deeper insights using additional protein markers available. We used IMC to perform deep phenotyping and spatial analysis of patient-matched primary, synchronous metastatic, and post-platinum-based chemotherapy HGSOC recurrence samples from 42 patients. This study represents the largest collection of highly multiplexed, spatially-resolved imaging data in HGSOC to date. We identified quantifiable spatial protein features of T cell and fibroblasts associated with early relapse (≤15 months after primary optimal debulking).

### Results:

### Imaging Mass Cytometry Reveals Single Cell Heterogeneity of Ovarian Cancer

We assembled a TMA composed of primary, synchronous metastatic, and metachronous recurrent tumor samples from 42 optimally debulked HGSOC patients who recurred during or after platinum-based chemotherapy (FIG. 38A in the priority U.S. provisional patent application No. 63/470,740). The time to recurrence ranged from 5.5 to 51.7 months after primary debulking surgery. Of the 42 patients, 16 recurred within 15 months following optimal primary debulking surgery, which we categorized as ‘early relapse’. The remaining 26 patients were categorized as ‘late relapse’. IMC analysis of the TMA was used to study the temporal evolution of spatial tumor architecture.

We performed IMC using an immune-centric panel of 38 markers (FIG. 38B in the priority U.S. provisional patent application No. 63/470,740), finding cell heterogeneity across all ROIs analyzed. Initially, major cell phenotyping markers were identified and used for Phenograph clustering (FIG. 38C in the priority U.S. provisional patent application No. 63/470,740), identifying three primary cell categories: immune cells (combined lymphoid and myeloid immune cell lineages), fibroblasts, and epithelial cancer cells (FIG. 38D in the priority U.S. provisional patent application No. 63/470,740). In addition to interpatient heterogeneity in cell composition, there was heterogeneity for each of the tumor sample types-primary, synchronous metastatic, and recurrent (FIG. 38E in the priority U.S. provisional patent application no. 63/470,740). Primary tumors had more epithelial cells and fewer immune cells than synchronous metastatic and recurrent tumors, but no statistically significant differences were observed. Significant differences were observed in cell proportions from lymph node metastases and recurrent tumors (FIG. 38F in the priority U.S. provisional patent application No. 63/470,740). ROIs from patients with early relapse (n=16) contained more fibroblasts and fewer cancer epithelial and immune cells than those from patients with late relapse (n=26), but these differences were not statistically significant.

### Deep Phenotyping Using IMC

Next, we performed fine-grained phenotyping of immune cells, identifying B cells (CD19+ or CD20+), plasma cells (CD138+/CD27+/CD38+), T cells (CD3+/CD4+ or CD8+), and macrophages and myeloid cells (CD68+, CD11b+) (FIG. 39A,B in the priority U.S. provisional patent application No. 63/470,740). T cells were further subdivided into CD4, CD8, and Treg (CD4+/FoxP3+) cells. Fibroblasts were further subdivided by Phenograph and labeled by expression of FAP, α-SMA, and podoplanin, or any combination of the three markers, to represent different fibroblast subtypes (FIG. 39C in the priority U.S. provisional patent application No. 63/470,740). Clustering of ROIs by their cell proportions identified an immune-dominant cluster (cluster 7) and a range of fibroblast- to epithelial-dominated clusters.

Macrophages comprised ˜45% of all immune cells while other myeloid cells comprised ˜21% (FIG. 39D in the priority U.S. provisional patent application No. 63/470,740). Different T cell subtypes were identified, including CD4, CD8, Treg, CD4/CD8 double-positive, and CD20-positive T cells. CD4/8 double positive and CD20+ T cells may represent densely packed T and B cells that were not separated by segmentation, although both cell types have been implicated in various cancers including ovarian. Plasma cells were difficult to define due to high CD138 expression on non-immune cells, such as epithelial tumor cells. Clusters were manually curated and validated by H&E. A subset of CD11b+ cells had to be re-classified from immune to epithelial cells as CD11b was expressed at high levels in a subset of epithelial cancer cells as previously observed by immunofluorescence and flow cytometry. The most common fibroblast cluster expressed FAP, α-SMA, and podoplanin, and fibroblasts expressing each combination of these markers were recorded (FIG. 39C in the priority U.S. provisional patent application No. 63/470,740). Ki67, a marker of proliferation, was expressed at high levels in epithelial cells, moderate levels in T and B lymphocytes, and low levels in macrophages, myeloid cells, and fibroblasts.

Patients presented a diversity of immune and fibroblast cell type distributions depending on the cell and tumor type. The proportion of T cells significantly increased from primary to recurrence samples (p=0.0038, Tukey's HSD) but not primary to metastasis (p=0.33), confirming previously reported results. As expected, tumors collected from lymph nodes had elevated lymphocyte numbers. Among fibroblasts, metastatic tumors had fewer α-SMA+ fibroblasts (metastasis to primary/recurrence, p=0.045/0.028), and recurrent tumors had fewer triple-positive fibroblasts (metastasis to recurrence, p=0.0024). Immune proportions were heterogeneous among patients, due in part to concentrated lymphocyte-rich regions.

### Spatial Microenvironmental Analysis Using IMC

To better characterize the spatial heterogeneity, we first analyzed the spatial distribution of major cell types-immune cells, fibroblasts, and epithelial cancer cells, across entire ROIs. We performed AGcross analysis to study how two hypothetical cell types A and B interact, with positive AGcross indicating that two cell types are separated and negative AGcross indicating close contact and mixing (FIG. 40A in the priority U.S. provisional patent application No. 63/470,740). We found self-interactions between cell types to be the most observed pattern. Using the average difference in expected to observed distance Gcross values for each ROI between immune cell, fibroblast, and epithelial cell distances as clustering variables, nine clusters of spatial organization were observed (FIG. 40B in the priority U.S. provisional patent application No. 63/470,740). Tissue patterns emerge from this clustering, such as ROIs with isolated immune or epithelial regions, or periodic patterns (FIG. 40B). Across tumor types, primary tumors were enriched in fibroblast isolated ROIs, and recurrent tumors were enriched in periodic structures and depleted in dispersed tumors. Immune isolated ROIs were rare, but enriched in samples of lymph node metastases. These spatially-informed clusters are different from the clustering generated by cell type proportions only.

We used spatial analysis to identify TLS, which have been associated with improved survival in ovarian cancer. Histologically, a TLS is defined as a lymphoid aggregate that contains a germinal center and high endothelial venules, however, these structures may be absent in thin histologic slices used for IMC. Since lymphoid aggregates are known to be enriched for T and B cells and depleted of epithelial cancer cells, we defined lymphoid aggregates by a second spatial analysis strategy, which generates a spatial enrichment score that reflects local cell concentrations at the single cell level (FIG. 40C in the priority U.S. provisional patent application No. 63/470,740). The lymphoid aggregate was defined as cells with combined T and B cell enrichment score >1 and epithelial enrichment score <0.8 (FIG. 40D in the priority U.S. provisional patent application No. 63/470,740). All cells satisfying these conditions were passed through a connectivity and size filter (>50 cells less than 15 μm apart from each other) resulting in detection of 93 lymphoid aggregates containing 569±1409 cells on average.

Using the spatial enrichment scores, we explored the composition of immune cells in different regions of the tumors. Lymphoid aggregates were the most prominent, well-defined collections of cells. Primarily composed of T cells and B cells, lymphoid aggregates had similar macrophage density to other stromal tissue, and lower tumor and fibroblast density. The immune composition in either epithelial-enriched or fibroblast-enriched regions was similar. We compared the relative spatial enrichment of macrophages vs T cells relative to epithelial cells, finding that epithelial cells in metastatic tumors were relatively enriched in T cells vs macrophages, despite the lower proportion of T cells than in recurrence tumors (primary to metastasis, p<1E-16, primary to recurrence, p=0.001).

Spatially Defined Cell Associations with Early Relapse

We explored whether severity of disease, as measured by time to relapse (<15 or >15 months), was significantly associated with tissue composition. Considering different tumor types independently (primary, metastatic, recurrent), no differences in cell percentages of fibroblasts, immune cells, epithelial cells, or immune cell subtypes were significantly associated with early relapse. Of the cell composition clusters, an FAP/α-SMA/podoplanin enriched fibroblast cluster was increased in early relapse ROIs (p=0.022, chi-squared test). No Gcross spatial clusters were associated with early relapse (p=0.72). Modeling the relationship of cell proportion measurements in entire ROIs with early relapse yielded two significant results associated with early relapse—the proportion of fibroblasts that were podoplanin-positive, and the proportion of FAP+α-SMA+ podoplanin+ triple positive fibroblasts (q<0.05). When primary, metastasis, and recurrence samples were analyzed individually, the fibroblast populations were found to be most significantly different in recurrence samples, while CD4 T cells were significantly associated with early relapse in primary tumors only.

We hypothesized that the spatial heterogeneity of tissue was conflated with cell proportion measurements, and that spatially informed analysis of immune, fibroblast, and epithelial cancer cell composition would provide more relevant or significant relationships with early relapse status. We first observed that in early relapse patients, epithelial cells were more spatially enriched for macrophages vs T cells (p<1E-16), indicating that cellular-level spatial analysis reveals differences where the overall composition of tissue does not. Then, we used the same spatial metrics used to define lymphoid areas to perform a “digital biopsy” by selecting subsets of tissue to compare between patients, specifically the immune-, fibroblast-, and epithelial-enriched zones. This allows for more effective “like versus like” comparisons between each ROI and addresses potential bias introduced by sampling tissue for TMA construction. Isolation of immune-, fibroblast-, and epithelial-enriched zones was performed using either a nearest neighbor or spatial enrichment threshold, with the nearest neighbor strategy being more sensitive to isolated epithelial tumor cells (FIG. 41A in the priority U.S. provisional patent application No. 63/470,740). Among all cell proportions measured in either immune-, fibroblast-, or epithelial-enriched digital biopsies, fibroblast-related metrics were the most significant terms after multi-test correction (FIG. 41B in the priority U.S. provisional patent application No. 63/470,740). A significant reduction in epithelial cancer cells and increases in fibroblasts in fibroblast zones were observed in early relapse patients (FIG. 41C in the priority U.S. provisional patent application No. 63/470,740). Compared to previous ROI-level metrics, the spatial biopsy method identified the specific areas of the tissue where the most changes were observed between patients with early and late relapse.

Fewer lymphoid aggregates were detected in early relapse patients, with an average of 1.56 lymphoid aggregates in early relapse ROIs and 1.91 in late relapse ROIs. Lymphoid aggregates also appeared to be smaller in early relapse patients (418±619 vs. 635±1,636 cells). Overall, lymphoid aggregates appeared in 52 out of 262 ROIs. After removing samples taken from lymph node metastases, lymphoid aggregates were detected in 38 of the remaining ROIs. ROIs from patients with early relapse averaged 1.42 aggregates (240±191 cells) while ROIs from patients with late relapse averaged 1.83 (300±634 cells). Since the lymphoid aggregates encompassed 73% of all B cells and 26% of all T cells, we analyzed the immune composition of ROIs outside of lymphoid aggregates to explore the contributions of more isolated, infiltrating immune cells. After comparing the significance of cell proportion comparisons with and without lymphoid aggregates, the fibroblast-associated significant associations remained significant, while other immune-related terms increased in significance but remained below the q<0.05 threshold.

We next considered the change in cellular composition between tumor types within patients to explore the spatiotemporal dimension of ovarian cancers. We hypothesized that the composition of different tumor types from the same patient could change by early relapse status. After measuring the change in each patient's tumor composition between tumor types (primary, synchronous metastasis, recurrence) with whole ROI-level metrics such as the total T cell percentage, we observed no significant changes. However, in all three digital biopsies, immune-enriched, fibroblast-enriched, and epithelial-enriched, we observed immune and fibroblast populations that were significantly associated with early relapse (FIG. 41D in the priority U.S. provisional patent application No. 63/470,740). These populations changed between tumor types within each patient according to the relapse status. For example, B cell percentages in fibroblast-enriched areas were greater in recurrent than primary tumors for late relapse patients, but the reverse trend was observed in early relapse patients (FIG. 41E in the priority U.S. provisional patent application No. 63/470,740).

### IMC and H&E Cell Morphology Concordance

Next, we compared IMC analysis to standard tools for pathology. Immune cells, fibroblasts, and epithelial cancer cells, were phenotyped by morphology in H&E-stained slides using QuPath. We then compared IMC phenotyping to cell morphology phenotyping. Two H&E sections were phenotyped by morphology, one proximal (˜20 μm away from the IMC section), and one distal (˜0.8 mm away from the IMC section) (FIG. 42A in the priority U.S. provisional patent application No. 63/470,740). We verified that our broad cell types were identified in each of the H&E sections and the IMC section. We found that cell composition in IMC was significantly correlated with the morphology-defined composition (FIG. 42B,C,D in the priority U.S. provisional patent application No. 63/470,740). This was true for both proximal and distal H&E sections, indicating that the cell composition was relatively consistent across the tumor. At low cell densities, IMC counts for fibroblasts were lower than H&E morphology counts, and vice versa for epithelial cells. One challenge in phenotyping by morphology is that immune cells were defined by small round cell morphology, which likely misclassified most macrophages as fibroblasts.

We compared the IHC analysis of whole slides to IMC using a CD8-specific stain to measure the percentage of immune cells that were CD8 positive (FIG. 42E in the priority U.S. provisional patent application No. 63/470,740). Generally, the whole slide count was similar to the counts for individual ROIs, although one core from patient 3 proved to be an exception. We further analyzed CD8 T cell composition using multiple methods for a subset of the cohort for which we had transcriptomic data. Using IMC, multiplex immunofluorescence (analyzed by TissueFAX), IHC (analyzed by QuPath), and NanoString gene expression, we found a high level of correlation across the different methodologies.

### Discussion:

Here we describe the largest highly-multiplexed spatial protein analysis of HGSOC currently available. Treatment of HGSOC presents a difficult challenge due to the high frequency of late-stage disease at initial diagnosis and the high rate of relapse with platinum-resistant disease following initial tumor debulking surgery and platinum-based chemotherapy. Patients might experience multiple relapses, but despite additional chemotherapy and/or surgery, the remissions typically become progressively shorter, ultimately resulting in treatment-resistant disease. The time to first relapse is a good indicator of tumor aggressiveness and overall survival. Using quantitative spatial protein technology, we confirmed published data about the relevance of immune cells, particularly lymphocytes in TLS, to patient outcomes. Further, we observed that podoplanin-positive fibroblasts were enriched in primary, synchronous metastasis, and recurrence samples from early relapse HGSOC patients. Podoplanin-positive CAFs have been described as facilitators of immunosuppression and cancer invasion in a variety of solid malignancies, and have been associated with disease progression and metastasis in ovarian cancer. Using spatial analysis, we now propose that podoplanin-positive CAFs are more influential and predictive of early relapse in the spatial context of other fibroblasts, rather than tumor or immune cells.

While single-cell RNA-seq analyses of ovarian carcinomas have confirmed the existence of transcriptomic signatures that define the major cell types previously inferred by bulk transcriptome analyses, those methods have not been able to capture the spatial context of the major cell types within ovarian cancer. Spatial communication between heterogeneous cell types in the tumor microenvironment could impact the efficacy of chemotherapy and immunotherapy, which can be seen in spatial transcriptomic analysis at 50 μm spatial resolution. CAFs and CAF-secreted ECM have been shown to reduce immune activity in solid malignancies by limiting T cell migration into the tumor parenchyma. In HGSOC patients treated with standard platinum-based chemotherapy, the immune-excluded phenotype tumors (in which T cells are accumulated in the stroma rather than in the tumor epithelium) had worse survival than the immune-desert phenotype tumors (in which T cells are absent or present in very low numbers), indicating that the tumor ecosystem and communication between fibroblasts and immune cells are key determinants of clinical outcome. Our analysis has shown that optimally debulked HGSOC patients with early and late relapse on standard platinum-based chemotherapy exhibit a distinct spatial configuration of epithelial cancer cells, fibroblasts, and immune cells without significant differences in the overall frequency of the individual cell types. This finding illustrates the advantages spatial analysis of the tumor microenvironment brings to understanding possible causes of early relapse.

As the number of single-cell methodologies has grown, a healthy skepticism has emerged regarding how well single-cell spatial methods can reproduce expert pathology analysis. A major advantage of IMC is the ability to discover new cell types expressing combinations of biomarkers due to its information content, which is only possible with highly-multiplexed spatial methods. However, a basic limitation to multiplexed image analysis is that segmentation of single cells remains a challenge, to precisely define cell boundaries without mixing membrane signals from neighboring cells. We encountered this challenge when quantifying CD4/8 and CD20+ T cells. We demonstrated that IMC reproduced many of the features that would be described by a pathologist examining H&E slides and that the IMC-H&E morphology agreement in defining major cell types held true for FFPE tissue section levels up to 1 mm from each other. Thus, the most significant spatial features of each tumor were relatively consistent over the distance studied.

Currently, H&E or IHC biomarker assays interpreted by pathologists are the norm in clinical settings. Due to tumor heterogeneity, using TMA core-sized ROIs (˜1 mm²) raises concerns of spatial bias. We used our digital biopsies to compare relevant subregions of tissue. The spatial analysis revealed that localized immune-epithelial-fibroblast associations have significant associations with early relapse, but the same bulk ROI-wide comparisons do not. As an analogy, spatially-informed analysis has the potential to improve current bulk ROI image analysis by adding local spatial context in the same way that single cell resolution improves bulk transcriptomic or proteomic measurements.

Our study represents a substantial advancement compared to the HGSOC IMC analysis published by Zhu et al. with respect to the number of patient samples (110 vs 41) and the number of ROIs analyzed (up to triplicates, 257 vs 41). In addition, our patient cohort was relatively homogenous, which allowed for robust comparison of longitudinally-collected tumor samples. All 42 patients were optimally debulked (absence of visible residual disease after surgery) and later relapsed with HGSOC. All primary tumors and synchronous metastases were collected before chemotherapy while all metachronous recurrent metastases were collected after 3-6 cycles of platinum-based chemotherapy. Notably, our study cannot be directly compared to the study by Zhu et al. due to the different cell subpopulations and different groups of patients analyzed. While the study by Zhu et al. compared cell densities in primary HGSOC samples from short-term survivors (overall survival ≤20 months) and long-term survivors (overall survival ≥60 months), we focused on the differences in the tumor microenvironment between HGSOC patients with early relapse (<15 months after optimal debulking) and late relapse (>15 months after optimal debulking) to better characterize patients who should be monitored for signs of relapse and receive more aggressive treatments. In some embodiments, a more aggressive treatment for those identified as having signs of relapse, especially those indicated/predicted to have early relapse by markers disclosed herein, include the introduction or a higher dose of chemotherapeutics such as cisplatin. In some embodiments, those identified as having signs of relapse, especially those indicated/predicted to have early relapse by markers disclosed herein, will be candidates for new therapies or new candidate therapies including clinical trial ones. Future studies will be performed using a larger multi-institutional patient cohort and finer-grained immune analysis as IMC panels achieve 45-plexed analysis currently.

### Techniques

Sample Selection. This study was approved by the Cedars-Sinai Institutional Review Board (IRB). Three types of tumors were collected from 42 patients-primary, synchronous metastatic, and metachronous recurrent tumors. Primary and synchronous metastatic tumors were acquired during primary debulking surgery (pre-chemotherapy) while metachronous/recurrent metastases were acquired during second-look surgery (post-chemotherapy). Primary ovarian tumors were collected from sites including the ovary, fallopian tube, or peritoneum, and synchronous and metachronous/recurrent metastases were collected from various intraperitoneal sites including the omentum, gastrointestinal organs, peritoneum or lymph node. A histologic diagnosis of HGSOC was confirmed in all tumor samples by pathology. After recovery from primary debulking surgery all patients were treated with 3-6 cycles of platinum-based chemotherapy; some were also treated with PARP1 inhibitors after recurrence. All patients in this study relapsed with HGSOC. Some patients had multiple remissions and recurrences. Metadata collected for each patient included the time to first recurrence, overall survival, age/stage/grade at diagnosis, race, and BRCA mutation status. The average age at diagnosis was 55.4 (range 45.3-62.1) years. Four patients had a prior history of cancer (two breast, one uterine, and one leukemia). The median disease-free interval (DFI) was 19.6 (range 5.5-51.7) months. Median overall survival (OS) was 65.9 (range 15.8-156.5) months. Four of 42 patients were alive at last follow-up (79.6, 91.2, 150.3, and 156.5 months). Forty of the 42 patients were diagnosed with stage III or stage IV disease. Of the two patients diagnosed with stage IIC disease, one was a BRCA1 mutation carrier and one was a BRCA1 and BRCA2 mutation carrier with previous history of breast cancer. Both patients had an aggressive course of the disease. All tumors, except one, were grade 3 serous papillary carcinomas. One patient was diagnosed with stage III, grade 1-2 papillary carcinoma arising from a borderline tumor. This patient had four recurrences with the first recurrence at 6.8 months of diagnosis, indicating rapid differentiation into HGSOC.

Tissue Microarray (TMA). In this Example, we define “sample” as tumor tissue obtained from a particular patient's primary, synchronous metastatic, or recurrent metastatic tumor, and “core” as a small circular region excised from a “sample” for further analysis. We generated a TMA with de-identified FFPE patient tissue samples comprising triplicate 1 mm cores of patient-matched primary HGSOC, synchronous pre-treatment metastasis, and metachronous post-treatment/recurrent metastasis samples (hereafter referred to as ‘primary’, ‘synchronous metastasis’, and ‘recurrence’, respectively) from 42 patients. Cores were extracted and assembled onto the TMA by manual selection by a histopathologist, comprising representative, tumor cell-rich regions of the samples. Each core was analyzed in full using IMC and referred to hereafter as a Region of Interest (ROI). Not every patient and timepoint was analyzed in triplicate due to ROIs either lost from the TMA or without tumor tissue. Manually-generated masks were used to exclude folded and/or necrotic tissue areas and staining artifacts. In total, 110 tumor samples (36 primary, 36 synchronous metastases, and 38 recurrences) and 267 total ROIs were analyzed, for an average of 2.42 ROIs per patient and timepoint available.

H&E Cell Morphology Phenotyping and Immunohistochemistry. H&E-stained TMA slides were digitized (40×) using the Aperio AT Turbo slide scanner from Leica Biosystems. Epithelial cancer cells, fibroblasts, and immune cells were phenotyped by morphology in digitized H&E-stained TMA slides using QuPath software for TMA analysis (TMA DeArrayer) and Random Trees (RTrees)-trained classifiers [70]. Immunohistochemistry using the CD8 antibody (clone JCB117, Ventana) was performed by the Cedars-Sinai Medical Center Biobank and Translational Research Core as previously described [71]. CD8 staining was assessed under the microscope by assigning scores: 0 (absent), 1 (1%-10%), 2 (11%-30%) or 3 (>30%) and by QuPath analysis using the Positive Cell Detection tool.

IMC Sample Preparation. Human tonsil and human tumor samples were used to optimize the immunostaining conditions. Antibodies were conjugated using MaxPar kits (Fluidigm) or directly purchased in conjugated form. FFPE slides were heated at 60° C. for 90 minutes then immersed in xylene for 20 mins. The slides were then subjected to 100%, 95%, 80%, and 70% ethanol washing steps for 5 minutes each. After washing with the alcohol gradient, the slides were immersed in Tris-EDTA antigen retrieval solution for 30 minutes at 95° C. and then left in the solution for 30 minutes at room temperature. After the antigen retrieval step, the slides were blocked with 3% BSA for 45 minutes and then stained overnight at 4° C. The next day the slides were washed twice with PBS-0.1% Triton X-100 solution and 1×PBS for 8 minutes each, incubated with 191 Iridium (a nuclear stain) for 40 minutes, washed with distilled water and then dried before ablation.

Data Acquisition and Processing. Data were acquired on the Hyperion/Helios Imaging Mass Cytometry platform (Fluidigm/Standard Biotools) at the Cedars Sinai Spatial Molecular Profiling Shared Resource. IMC data was acquired at an acquisition speed of 200 Hz. Single cells were identified using the ilastik random forest pixel classification program. Multiple markers for cell nuclei and membrane/cytosol were used to add redundancy to the classification, and single-cell masks were verified by visual inspection. Artifacts and defects in segmentation, such as folded tissues or necrotic regions, were identified and manually excised from each ROI. Antibodies with poor or non-specific staining were excluded from the final analysis. Cells were filtered for size and those with an area <15 μm² or >500 μm² were removed. Signal for each protein was arcsinh transformed, censored at the 99th percentile, and scaled from 0 to 1. ROIs with fewer than 500 cells total were removed from the analysis, leaving a total of 1,578,369 cells from 267 ROIs for analysis, not including control tissues. Cell phenotypes were obtained using Phenograph clustering (k=15) followed by manual annotation.

Spatial Analysis. Multitype nearest neighbor distribution (Gcross) analysis was performed using the spatstat R package. Actual observed and expected nearest neighbor distances between two cell types were calculated, distances were normalized to 50 μm, and the difference between the expected and observed distances was obtained. In a Gcross differential plot between two different cell types A and B, a positive value for the Gcross differential indicates that there are fewer B cells close to A cells than would be expected in a random distribution; thus the two cell types are segregated in space. A negative differential indicates that the cell types A and B are more intermixed or paired together than a random distribution would predict. A negative A-B differential (B cells are intermixed with A cells) does not necessarily result in a negative B-A differential based on the cell proportions and distribution. For a Gcross differential plot within a single cell type, A to A, a negative differential denotes cell clustering and a positive differential denotes more regular, distant spacing between cells of type A.

The “digital biopsy” method is defined by preselecting specific cells for analysis by a spatial condition. To perform spatial analysis using the digital biopsies and to identify lymphoid aggregates, we performed a modified nearest neighbor analysis. For every cell in each ROI, we calculated an enrichment score based on that cell's proximity to each of the three cell subtypes of interest (immune, epithelial, fibroblast). The score is calculated by measuring the average distance of each cell to its 5 nearest neighbors of the specified cell type, capped at 100 μm. The average of these distances is scaled from 0-1 by dividing by 100 μm and subtracted from 1, such that 0 indicates minimal interaction between the two cell types. Three primary cell interaction scores—an immune spatial, cancer epithelial spatial, and fibroblast spatial score are calculated for each cell, as well as interaction scores with immune subtypes such as T cells and macrophages.

Modeling and Statistics. To test for statistical significance in Gcross differences by early and late relapse status, the glm.cluster function in the R miceadds package was used. For each type of comparison (immune-immune, immune-fibroblast, etc.), the Gcross data consisted of the difference between the observed nearest neighbor and the actual difference (y-axis data), measured by the distance from each cell at increments of 1 μm up to 50 μm (x-axis data). Using the x-axis distance as the clustering variable, we calculated the significance of refractoriness using a generalized linear model to determine if the spatial tissue composition was significantly associated with refractoriness when considering the tumor type (primary, synchronous metastasis, recurrence). Multi-test correction using the Benjamini-Hochberg procedure was performed for testing the significance of cell proportions towards predicting early relapse. For comparisons within patients across timepoints, the absolute difference in cell proportions was used and not the relative change, and multi-test correction was not applied due to high variability in scale of absolute cell differences. All patients were women, and the average age at diagnosis of early and late relapse patients was 56.0±10.8 and 55.3±8.9 years, respectively.

Software and Packages. Analysis was performed using R version 4.2.1 and Rstudio 2022.07.1. Rstudio packages Rcpp, Rphenograph, data.table, tidyverse, gridExtra, pheatmap, uwot, spatstat, viridis, tidyr, stringi, parallel, doParallel, EBImage, ggplot2, ggbeeswarm, miceadds, plotly, and RColorBrewer were used. Images were generated using MCD Viewer (Fluidigm/Standard Biotools). IHC analysis was performed using QuPath in which cell type classifiers were trained using Random Trees.

TABLE 1
Patient demographic and clinical data in this Example.

| | | All Patients | 16 Early | [range] | 26 Late | [range] |
|---|---|---|---|---|---|---|
| | | [range] | relapse | or % | relapse | or % |
| Median age at | Years | 54.08 | 55.46 | [39.52-74.59] | 53.26 | [39.42-71.12] |
| diagnosis | | [39.42-74.59] | | | | |
| Median time to | Months | 19.63 | 9.18 | [5.5-13.47] | 26.87 | [17.3-51.7] |
| first relapse | | [5.5-51.7] | | | | |
| Median overall | Months | 65.9 | 31.63 | [15.83-136.8] | 79.6 | [36.2-156.5] |
| survival | | [15.8-156.5] | | | | |
| Race/Ethnicity | Caucasian | 33 | 11 | 68.75 | 22 | 84.62 |
| | African | 3 | 2 | 12.5 | 1 | 3.85 |
| | American | | | | | |
| | Asian | 3 | 0 | 0 | 3 | 11.54 |
| | Latino | 2 | 2 | 12.5 | 0 | 0 |
| | Other | 1 | 1 | 6.25 | 0 | 0 |
| Primary tumor | Ovary | 38 | 15 | 93.75 | 23 | 88.46 |
| location | Fallopian | 2 | 1 | 6.25 | 1 | 3.85 |
| | tube | | | | | |
| | Peritoneum | 2 | 0 | 0 | 2 | 7.69 |
| Stage at | IIC | 2 | 0 | 0 | 2 | 7.69 |
| diagnosis | III | 33 | 13 | 81.25 | 20 | 76.92 |
| | IV | 7 | 3 | 18.75 | 4 | 15.38 |
| Grade at | 3 | 41 | 15 | 93.75 | 26 | 100 |
| diagnosis | 1-2 | 1 | 1 | 6.25 | 0 | 0 |
| BRCA1/2 | Not tested | 18 | 12 | 75.00 | 6 | 23.08 |
| mutation | WT | 14 | 4 | 25.00 | 10 | 38.46 |
| status | BRCA1 | 6 | 0 | 0.00 | 6 | 23.08 |
| | BRCA2 | 2 | 0 | 0.00 | 2 | 7.69 |
| | BRCA1/ | 2 | 0 | 0.00 | 2 | 7.69 |
| | BRCA2 | | | | | |

TABLE 2
List of antibodies and their corresponding clones and metal tags
used for IMC. Markers not used in analysis are denoted with *.

| Antibody | Clone | Metal Tag |
|---|---|---|
| CD45 | D9M8I | Y89 |
| FAP | Polyclonal | In115 |
| | (Thermo Fisher | |
| | PA5-51057) | |
| HLAABC | EMR8-5 | La139 |
| CD38 | EPR4106 | Pr141 |
| CD11c | 118/A5 | Nd142 |
| NKG2A | Polyclonal | Nd143 |
| | (Thermo Fisher | |
| | PA5-21949) | |
| α-SMA | 1A4/asm-1 | Nd144 |
| Tbet | D6N8B | Nd145 |
| CD16 | EPR16784 | Nd146 |
| BCL6* | K112-91 | Sm147 |
| PanKeratin | C11 | Nd148 |
| CCR4 | 205410 | Sm149 |
| CD68 | KP1 | Nd150 |
| CD138 | Polyclonal | Eu151 |
| | (Thermo Fisher | |
| | 36-2900) | |
| TGFβ | 1D11.16.8 | Sm152 |
| LAG3 | D2G40 | Eu153 |
| CXCL13* | Polyclonal | Sm154 |
| | (Biotechne | |
| | AF801) | |
| CD11b | EPR1344 | Gd155 |
| CD4 | EPR6855 | Gd156 |
| HLADPDQDR | CR3/43 | Gd158 |
| PDL1 | 28-8 | Tb159 |
| CD20 | H1 | Dy161 |
| CD8A | C8/144B | Dy162 |
| FoxP3 | 236A/E7 | Dy163 |
| RoRgT | 6F3.1 | Dy164 |
| PD1 | NAT105 | Ho165 |
| CD19 | 109 | Er166 |
| Granzyme B | EPR20129-217 | Er167 |
| Ki67 | B56 | Er168 |
| CD3 | Poly-C-term | Er170 |
| CD27 | EPR8569 | Yb171 |
| PDL2 | 176611 | Yb172 |
| IDO | mIDO-48 | Yb173 |
| Podoplanin | D2-40 | Yb174 |
| IL10* | PM1121 | Lu175 |
| DNA | | Ir191 |
| DNA | | Ir193 |
| Histone H3 | EPR16987 | Bi209 |

### Example 7. (Imaging Mass Cytometry-Driven) Single Cell Spatial Analysis and Biomarker Discovery in Hodgkin Lymphoma

### Abstract:

The biology of tumors is suffused with spatial interactions, such as tumor-immune signaling through localized cytokine/ligand secretion, cell-cell contacts, and checkpoint ligand/receptor signaling. Hodgkin Lymphoma (HL) can serve as a study paradigm for tumor microenvironment (TME) architecture as the defining pathological feature is the scarcity of the malignant Hodgkin and Reed Sternberg (HRS) cells, leaving a diverse and predominantly immune cell rich tumor microenvironment (TME) with complex tumor-immune interactions. Previous studies have identified TME features that are prognostic and predictive, however these studies did not consider the entirety of TME cellular ecosystems, including precisely defined immune cell subsets with opposing inflammatory and immune-suppressive effects, as a determinant for differential clinical course of HL patients. Here we use Imaging Mass Cytometry (IMC) with 42 antibody markers to profile tumors from 93 patients with HL. Our cohort consists of relapsed/refractory HL with matched diagnostic and relapsed biopsies, and we present a bioinformatic pipeline to profile 10 major cell lineages and their subtypes including spatial interaction mapping. Our pipeline identifies putative biomarker candidates with a focus on “rosettes”-local aggregates of immune cells around single tumor cells. In addition to validating existing biomarkers centered on CD68+ macrophages, GranzymeB+CD8+ T cells, and others in HL, we propose new biomarkers based on localized interactions between HRS cells and aggregating CD4+ and CD8+ T cells and macrophages involving the immune checkpoints PD1/PDL1, LAG3, and Galectin9. This study serves as a broad tissue imaging resource for multi-timepoint biopsies in HL, and a computational resource and pipeline for users of IMC and other multiplexed imaging studies to perform tissue analysis and biomarker candidate testing with any tissue type.

### Background:

Spatial analytics is an essential tool in the clinical oncology repertoire and a common diagnostic method for diagnosing cancer and predicting outcomes when used in pathology and immunohistochemical (IHC) staining. Image interpretation is performed by trained pathologists, who integrate many factors including cell and nuclear morphology, staining intensity, and spatial context of cells. These observations guide clinical decisions, based on the tumor structure, risk factors, and treatment options available. By comparing a single tissue stain to the historical record of tissue accrued over time, patterns in the tissue can be associated with patient survival and clinical outcomes after treatment. These patterns, along with clinical and molecular data with similar predictive power, are called biomarkers.

Tumors exhibit a wide range of host immune response which is reflected in the numbers of immune cells which are found surrounding and infiltrating the malignant cells. Hodgkin Lymphoma (HL), featuring an immune-rich tumor-microenvironment (TME), is composed of a multitude of non-malignant cell types educated by malignant Hodgkin and Reed-Sternberg (HRS) cells, which are relatively isolated and comprise <10% of the tissue. The pattern and structure of the TME has been used to classify Hodgkin lymphoma into histological subtypes—nodular sclerosis, mixed cellularity, lymphocyte-rich and lymphocyte depleted; however, these spatial classifications have little prognostic impact and are not relevant to initial treatment selection. Instead, prognoses are made using clinical variables such as International Prognostic Score or positron-emission tomography. Standard treatment of primary HL consists of doxorubicin, bleomycin, vinblastine, and dacarbazine (ABVD), and recent clinical trials confirmed the benefit of an additional CD30 targeting treatment, brentuximab vedotin, in advanced HL. Despite these treatment advancements, about 20-30% of patients still experience relapse within a year or are refractory to treatment. About half of relapsed/refractory HL patients can achieve long term remission after high dose chemotherapy followed by autologous stem cell transplantation.

Previous work in HL has that indicated specific cell types such as FoxP3+ T cells, macrophages, and many others have prognostic value, but the spatial context of these cells was overlooked. Recently, HL-specific spatial patterns have been explored in new ways using multiplexed imaging technologies. PDL1 overexpression is common in HL tumor cells due to chromosomal translocations, and the recent use of multiplexed imaging studies has shown that PDL1+ macrophages and monocytes are found closer to PDL1+ HRS cells, where they interact with PD1+CD4+ T cells. Other checkpoints such as CTLA4 follow their own spatial patterns of expression in the TME. A study of 108 cases reported that the relative MHCI expression on HRS cells to their immediate neighbors predicted the results of chemotherapy. The observation that HRS cells recruit other immune cells, especially CD4+ T cells, to form aggregates called “rosettes”, indicates that spatial analysis of HRS-immune cell neighbor interactions is significant.

Here we present one of the largest collections of HL reported and analyzed with highly multiplexed, single cell, spatial protein analysis with subcellular resolution. A total of 93 HL patients were analyzed, including 73 patients with relapsed/refractory disease and an additional 20 patients with no relapse. For each patient that relapsed, paired diagnostic pretreatment and relapse tumor tissues were profiled.

Herein, our formalin-fixed paraffin embedded (FFPE) tissue data was analyzed using Imaging Mass Cytometry (IMC) to measure 35 proteins at 1 μm resolution. We present a detailed analytical pipeline to process and analyze these image data. We performed deep phenotyping and spatial analysis to describe the tumor architecture and functional protein expression profiles of major cell types, and we emphasized hyper-local protein expression by extracting patterns of immune checkpoint ligand-receptor co-expression between HRS and immune cells. Finally, we present a computational pipeline to propose biomarker candidates using IMC data, which have both protein and spatial components. We explored specific biomarkers using Cox survival analysis, and we also provide a tool to generate biomarker combinations based on a variant of the least absolute shrinkage and selection operator (LASSO), tailored to different spatial tumor contexts.

### Results

### Hodgkin Lymphoma Preliminary Analysis

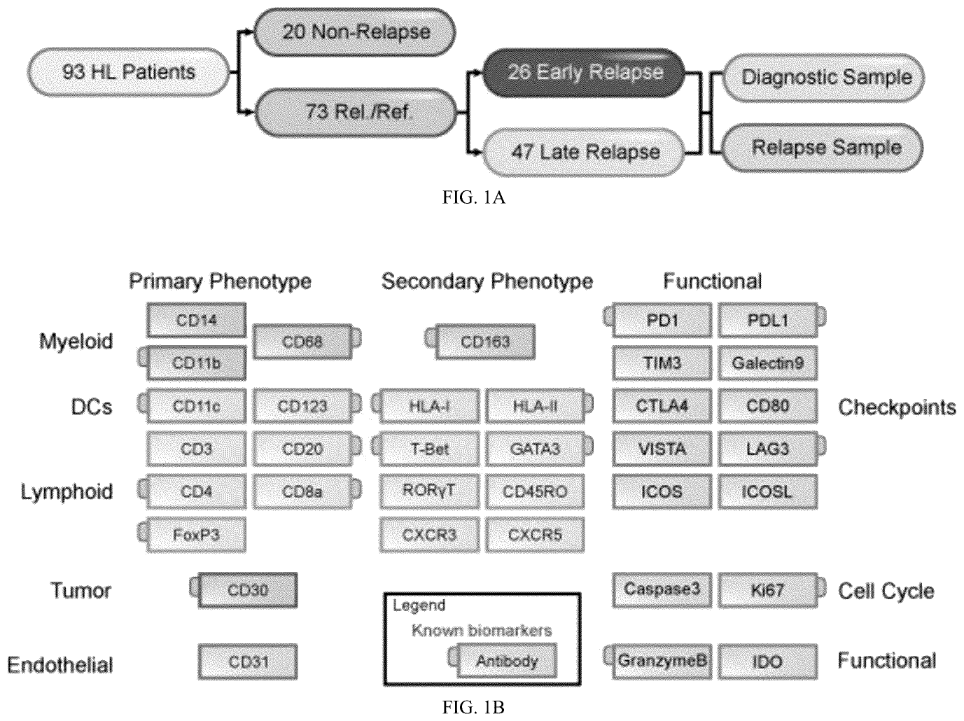

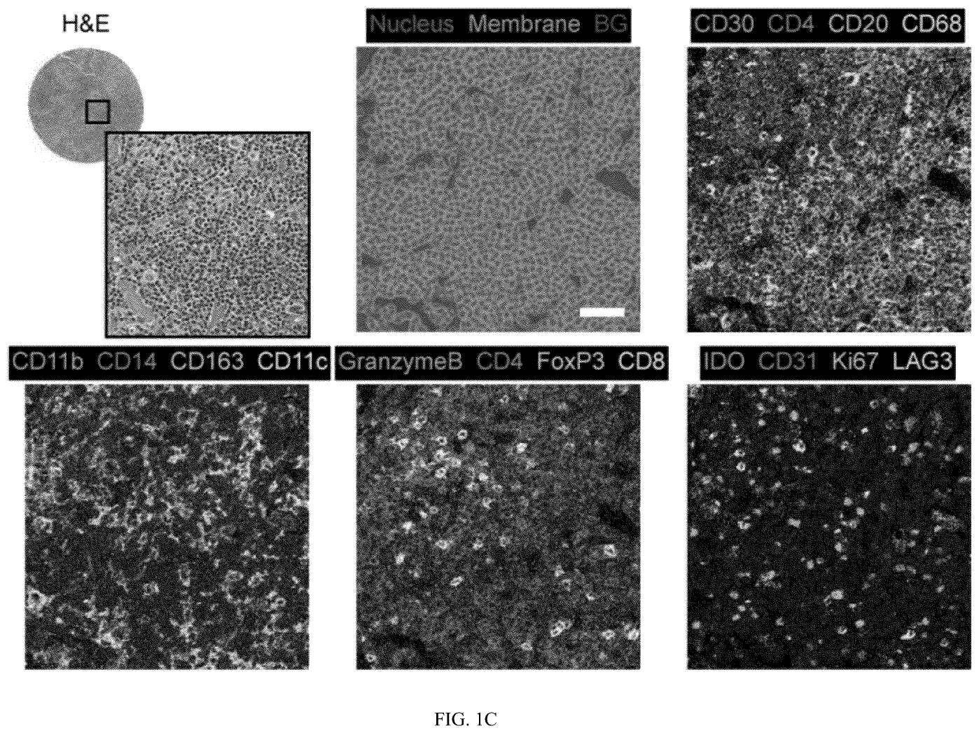

We studied a cohort of 93 total HL patients comprising 261 regions of interest (ROIs) assembled on tumor microarrays (TMAs) with up to 2 timepoints per patient and up to 2 ROIs per timepoint when available (average 1.76). We selected a cohort containing samples from patients with variable relapse status, including 20 non-relapsed patients and 73 relapsed patients, of which 26 relapsed within 1 year (‘early relapse’) and 47 relapsed later than 1 year (‘late relapse’) (FIG. 1A). All relapsed patients had matched samples from diagnostic pretreatment and relapse time points, permitting comparisons over time. The panel used for HL analysis consisted of primary phenotyping lineage markers, secondary lineage markers, and functional or inducible markers (FIG. 1B). Tumor cells as defined by CD30 along with immune cell types were first identified, and cell subtypes were then selected based on checkpoint expression. Proteins reported as biomarkers or potential therapeutic targets are denoted in FIG. 1B. Our cohort was designed to identify relapse-specific biological features and develop biomarker assays to improve clinical decision making. Our specific marker panel reflected the demonstrated and growing importance of the TME and immune checkpoints in HL treatment. Specifically, myeloid has primary phenotyping lineage markers of CD14, CD11b, and CD68, and secondary phenotyping lineage markers of CD163; dendritic cells (DCs) has primary phenotyping lineage markers of CD11c and CD123, and secondary phenotyping lineage markers of HLA-I and HLA-II; and lymphoid has primary phenotyping lineage markers of CD3, CD20, CD4, CD8a, and FoxP3, and secondary phenotyping lineage markers of T-Bet, GATA3, RORYT, CD45RO, CXCR3, and CXCR5; and tumor cells identified by CD30; endothelial cells identified by CD31; and subtypes categorized based on expression of checkpoint proteins including PD1, PDL1, TIM3, Galectin 9, CTLA4, CD80, VISTA, LAG3, ICOS, and ICOSL, OR based on cell cycle status proteins including Caspase3 and Ki67, OR based on functional proteins including GranzymeB and IDO.

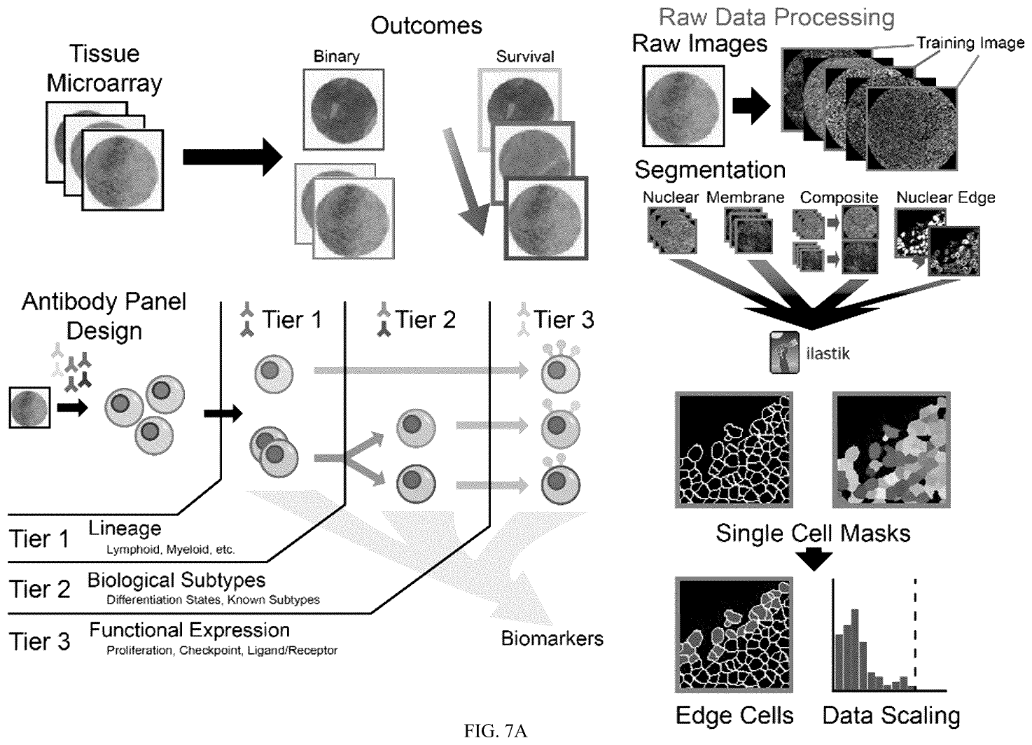

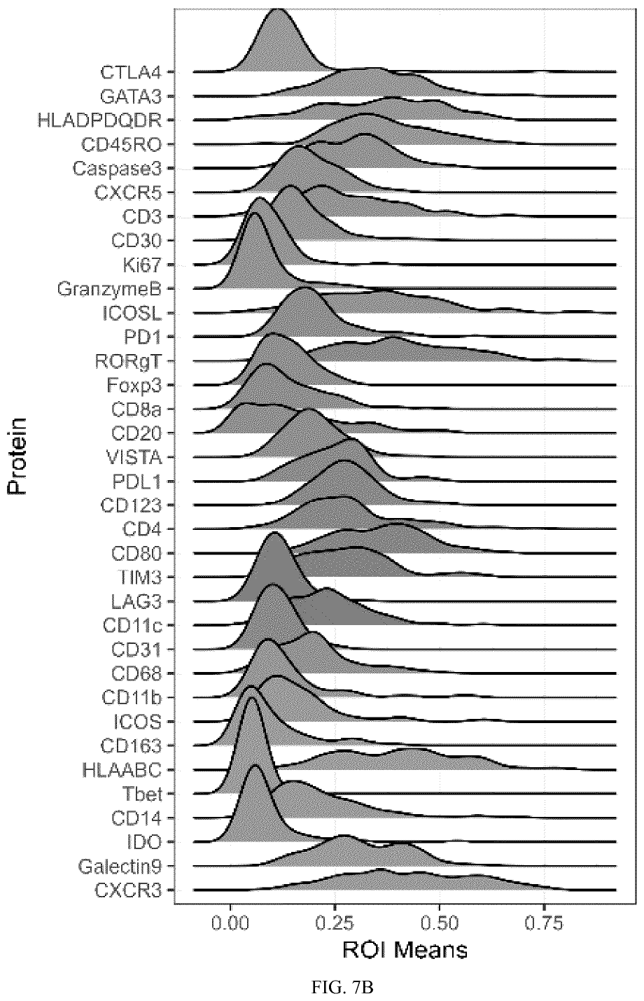

We analyzed 7.05M cells total using our spatial analytic pipeline (FIG. 7A), for an average of 27,002±9,754 cells per sample, and 46,984±22,085 cells per patient timepoint. Average marker expression in cells was approximately normally distributed across ROIs (FIG. 7B). Representative images showed a heterogenous immune microenvironment with classic HL morphology, consisting of large HRS cells embedded in a matrix of immune cells in the TME (FIG. 1C). Segmentation partitioned each image into nuclei, cytoplasm/membrane, and background areas used to identify single cells, and protein expression patterns specific to lymphocytes, myeloid cells, and other functional markers are shown. All subsequent analysis was performed on single cells using average protein expression of each marker.

### HL Immune Phenotyping

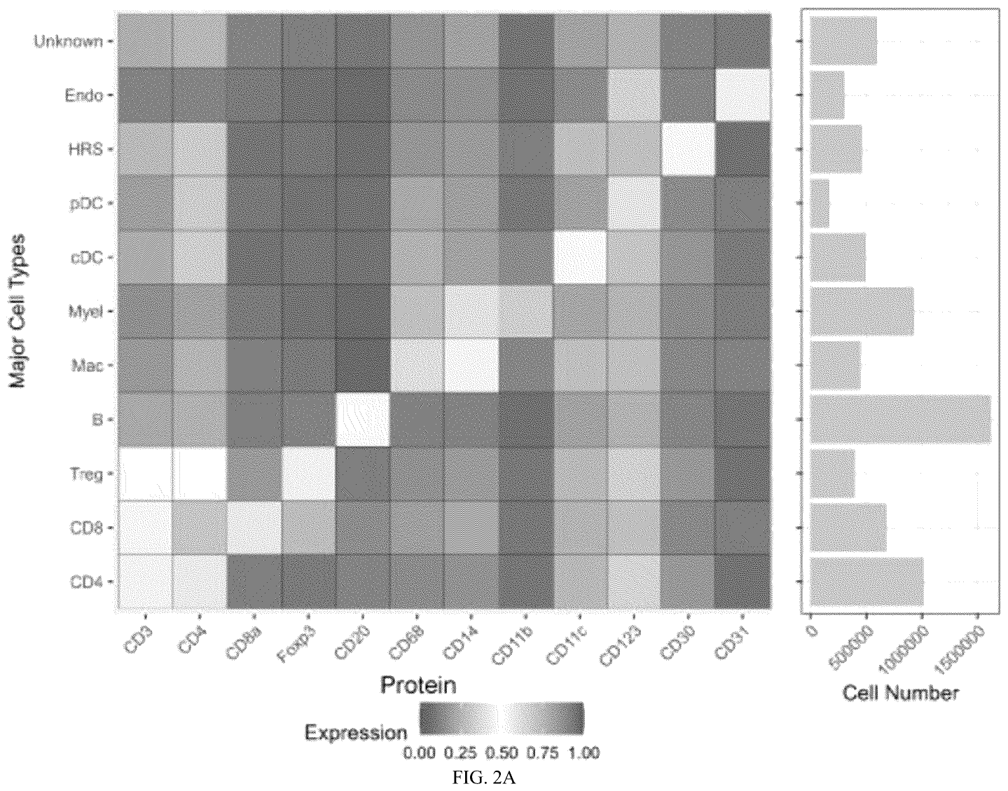

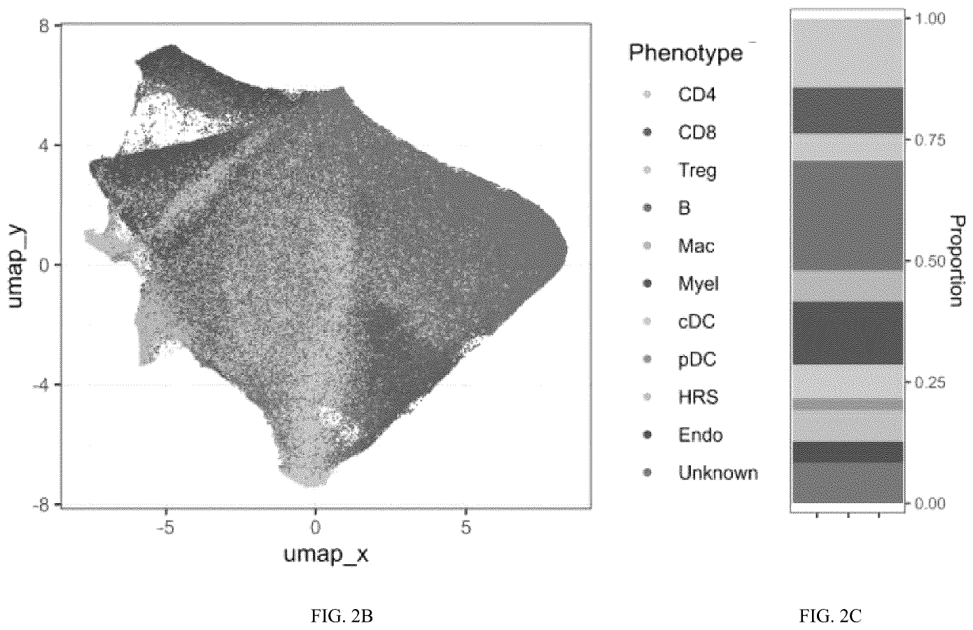

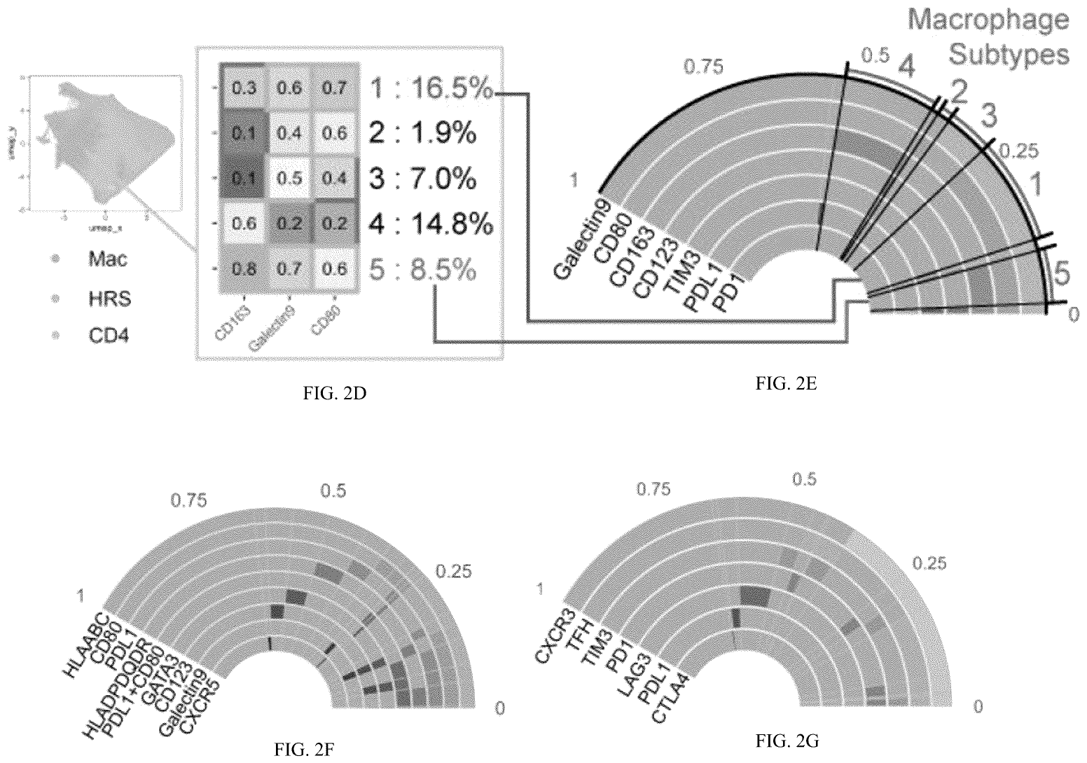

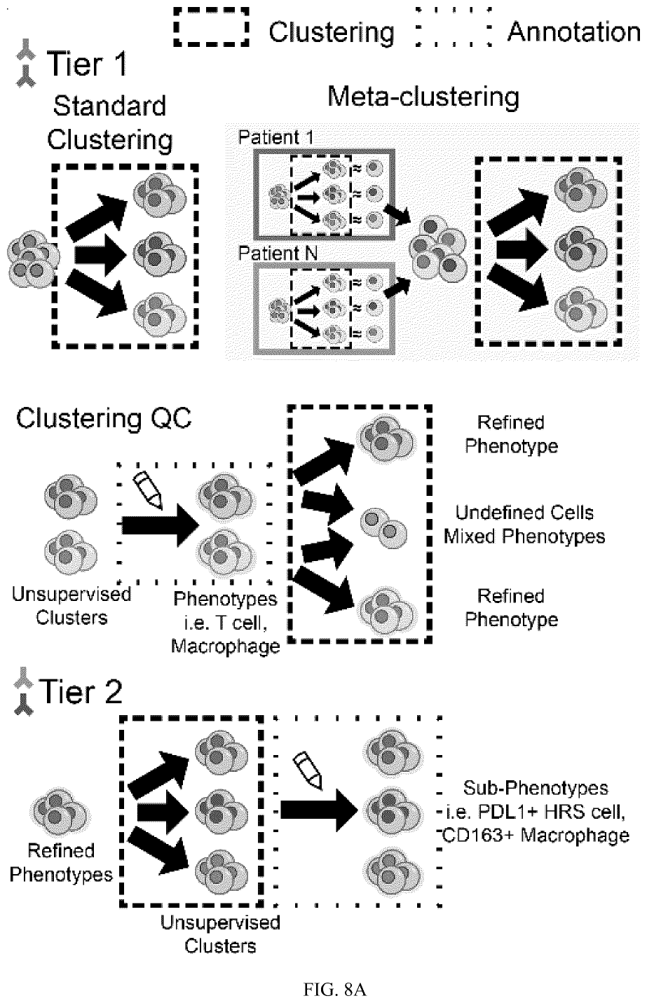

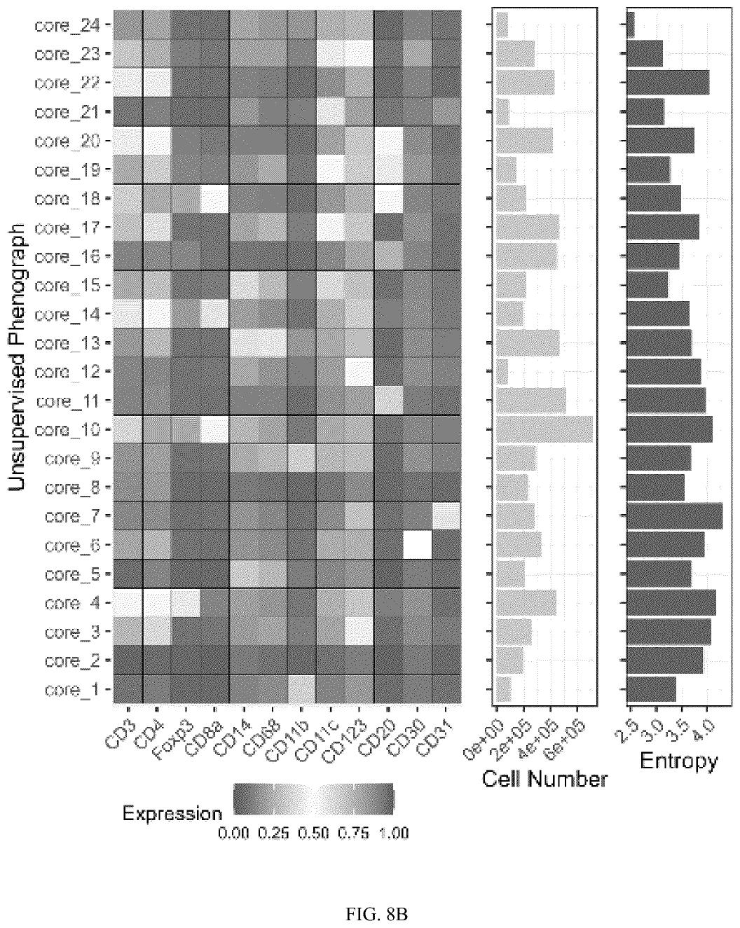

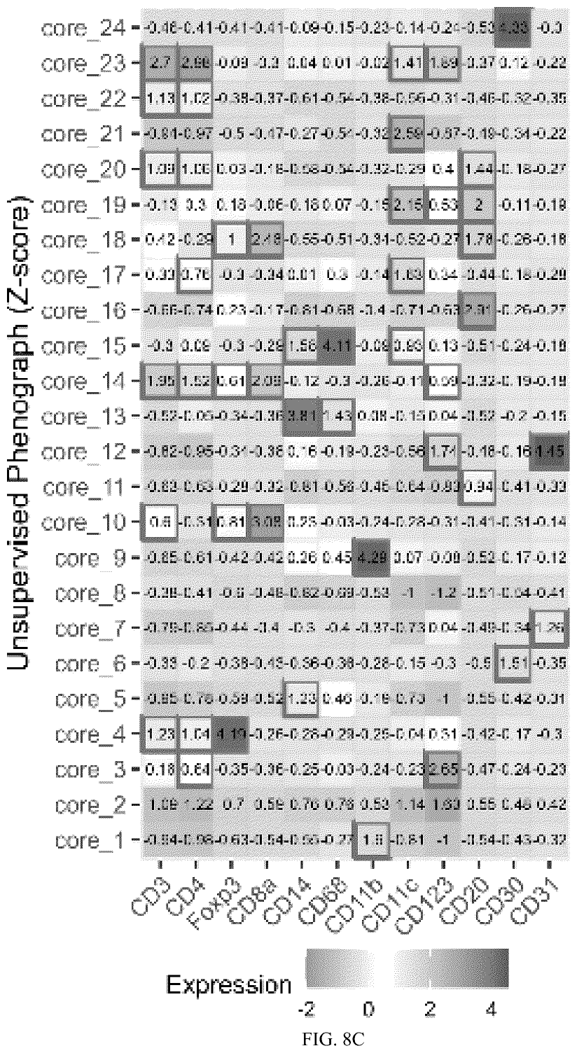

We identified 10 major cell types using hybrid hierarchical and manual metaclustering (FIG. 8A-8E): T cells (CD4+, CD8+, Treg), B cells, macrophages and other myeloid lineage cells, conventional and plasmacytoid dendritic cells, endothelial cells, and HRS tumor cells (FIG. 2A). In a UMAP projection (downsampled to 10%), cell clusters were crowded due to the densely-packed lymphoma tissue and imperfect segmentation (FIG. 2B). Across patients, major cell types were heterogeneously distributed. The largest proportion of cells were T cells (˜31%) split into CD4+ (16%), CD8+ (11%), and Treg subtypes (6%), B cells (23%), and myeloid cells (20%, 6.5% macrophages). Dendritic cells (7% conventional, 2.5% plasmacytoid), tumor cells (7.5%), and endothelial cells (4.5%) comprised the remainder of the tissue (FIG. 2C). The 7.5% abundance of HRS tumor cells we observed was likely due to our emphasis on relapsed/refractory tumors, which are enriched in HRS cells. Out of 7.05M cells, ˜6% were found in clusters that were not well-defined by a single phenotype. Of these, 382k expressed multiple canonical cell phenotyping markers, which were denoted as “mixed” cells. For these difficult-to-define cells, each cell was labeled with as many descriptors as applicable (i.e. CD4+ T cell, macrophage), which were not mutually exclusive to account for the possibility that two cells were overlapping in the same section of the tissue slice.

Within immune cell types, additional clustering was performed to identify relevant subtypes. Similar to the mixed cell process, we adopted a strategy of labeling cells with as many subtypes as applicable, where a subtype was defined by 1-2 protein markers (FIG. 2D-2G). To illustrate this strategy, a heat map of a macrophage subset expressing CD163, Galectin9, CD80, and their combinations is shown (FIG. 2D). The proportion of these 5 clusters relative to all macrophages is shown in FIG. 2E, where macrophages co-expressing Galectin9 and CD80 (label 1) or all three markers (label 5) were common while CD80+ only macrophages (label 2) were relatively rare. Ki67+ proliferative cells comprised 8% of all cells but 22% of HRS and 16% of Tregs. HRS cells (11%), macrophages (16%), and CD8+ T cells (14%) were more apoptotic (Caspase3+) compared to all cells (8%). Among T cell subtypes, CD45RO expression, which denotes a memory subtype, was increased in PD1+ and TIM3+ T cells, and decreased in LAG3+ T cells. Our phenotyping approach was single-marker-focused to facilitate downstream biomarker analysis, where subtypes such as PDL1+ HRS cells can be recalled easily but complex phenotypes were still identified via clustering.

### Spatial Analysis

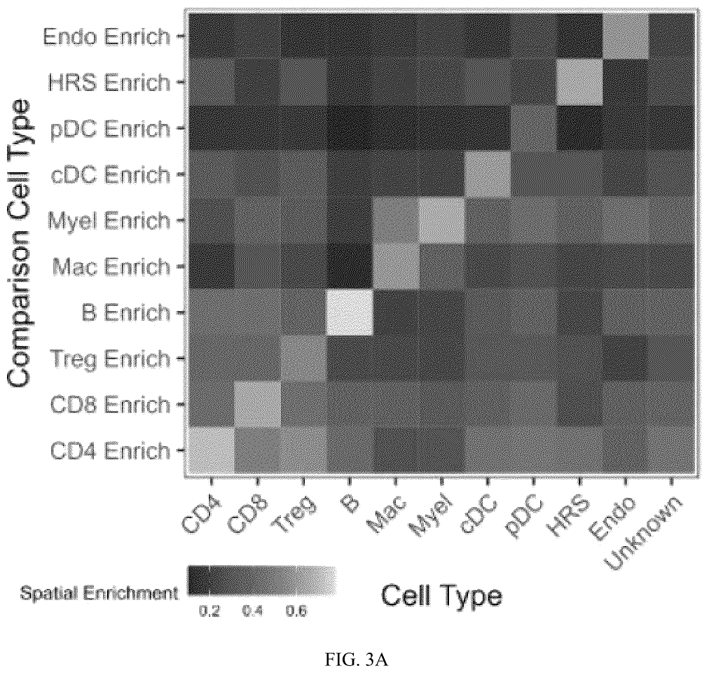

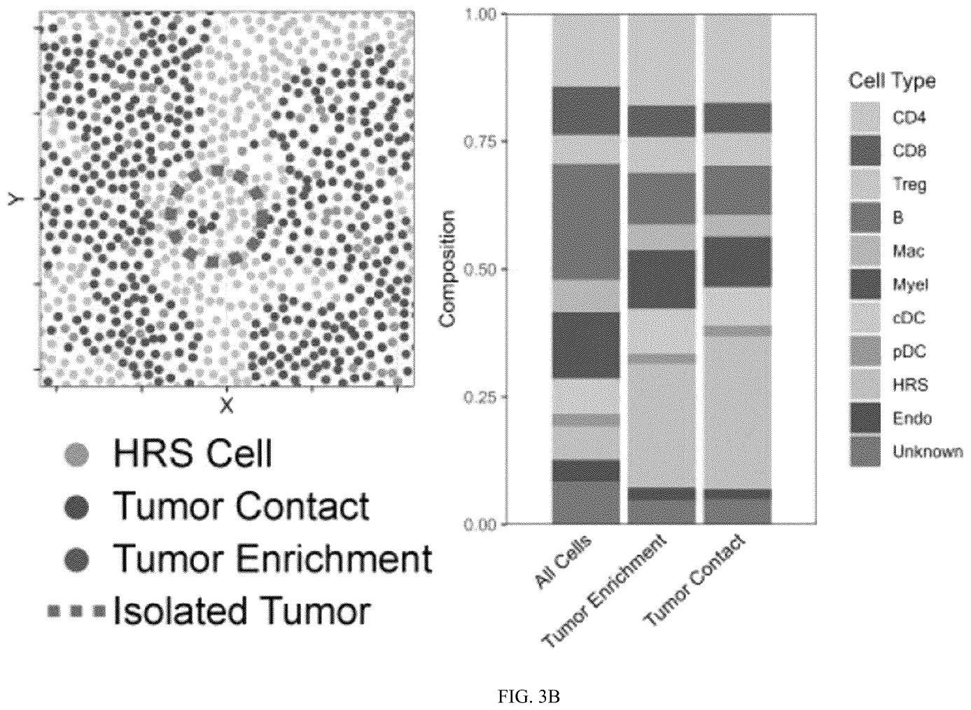

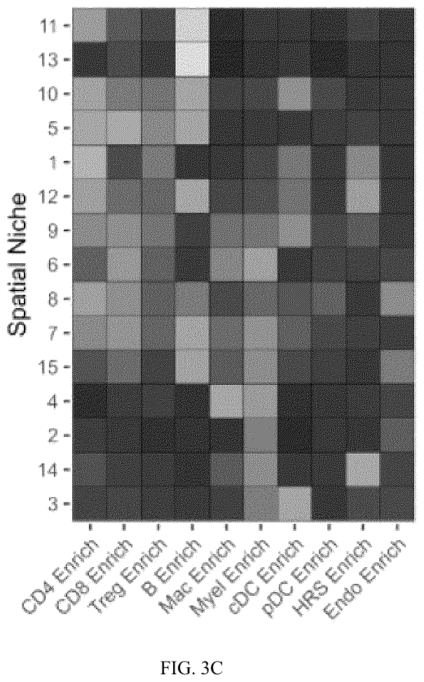

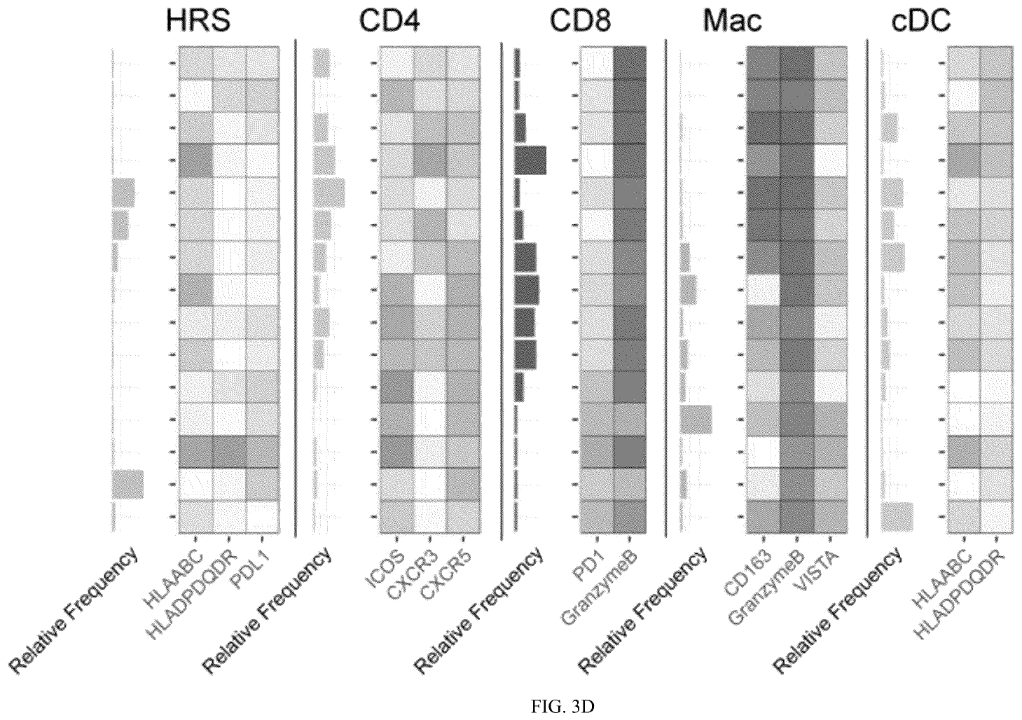

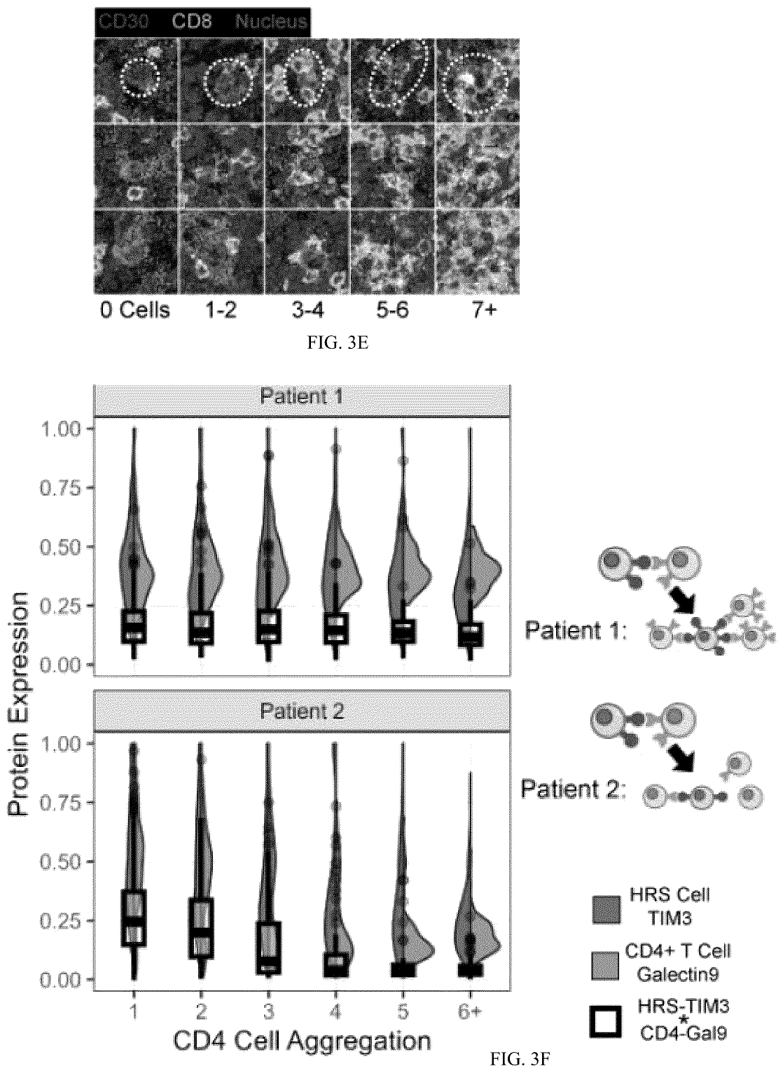

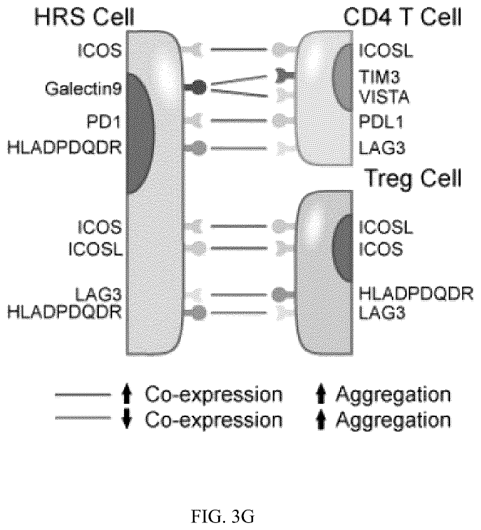

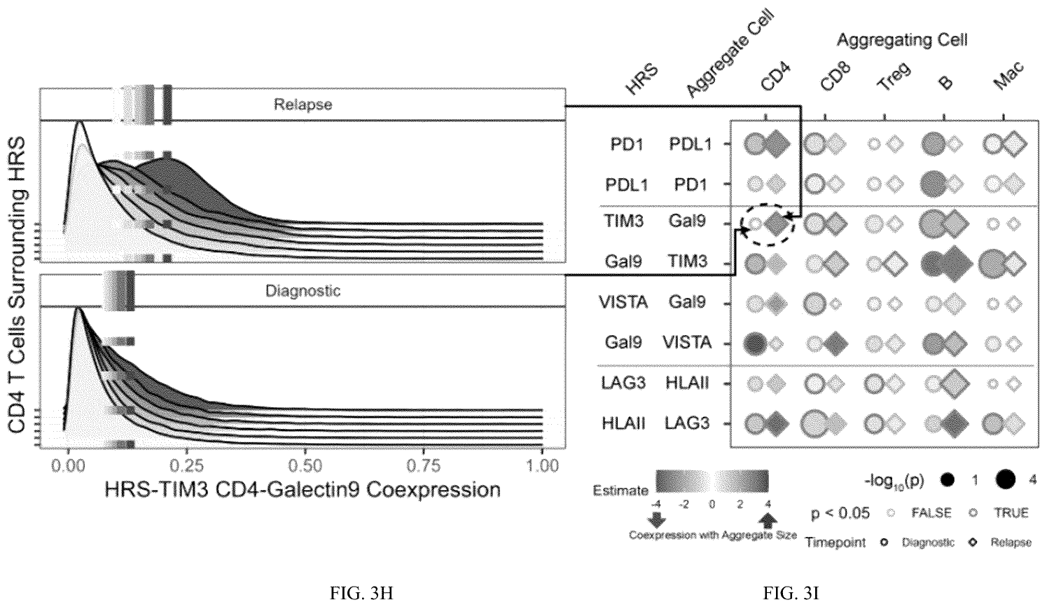

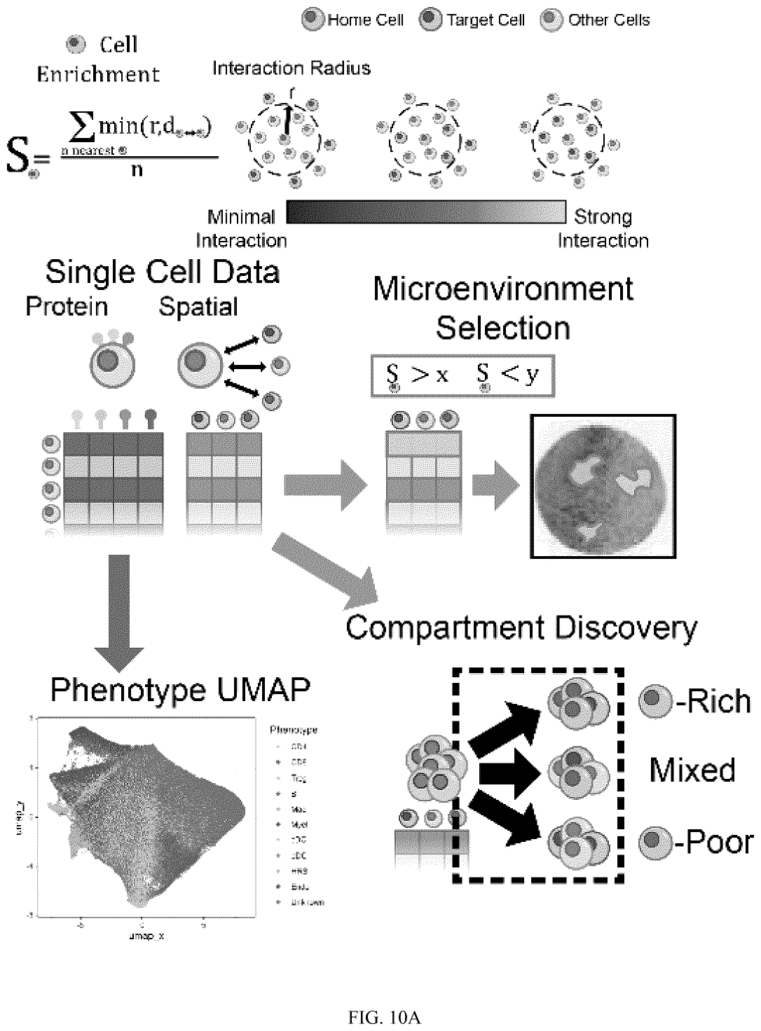

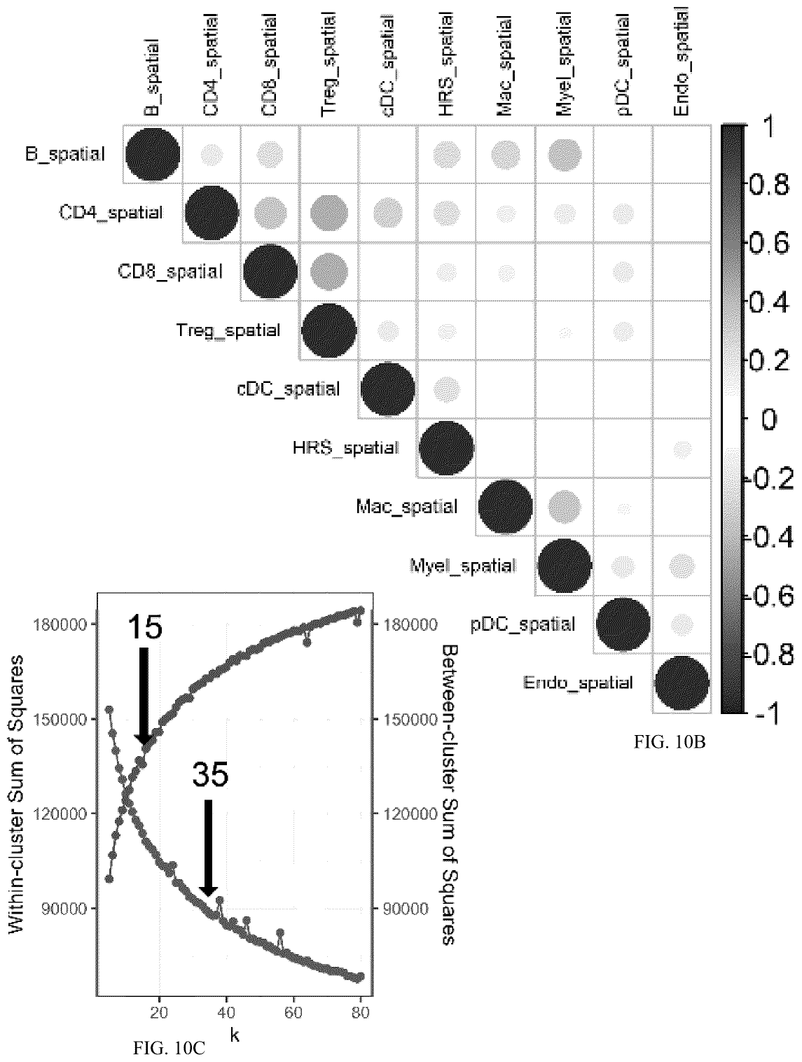

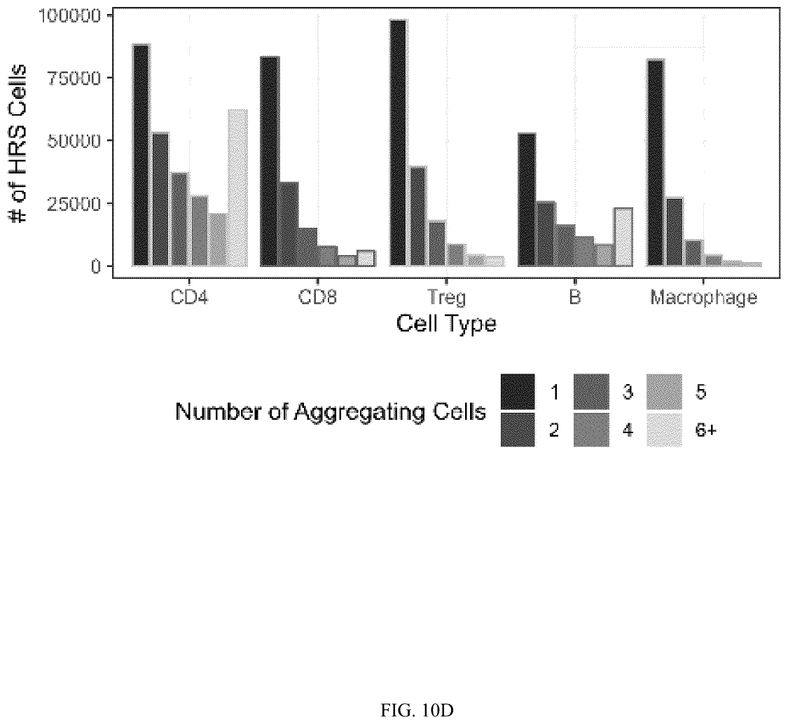

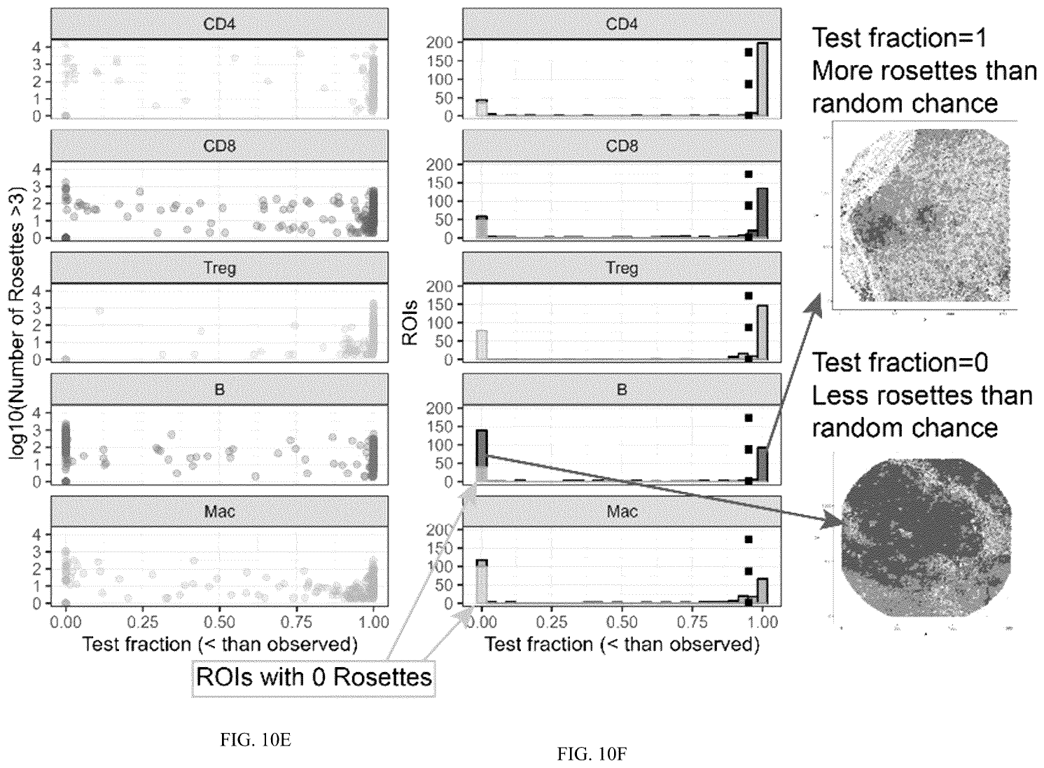

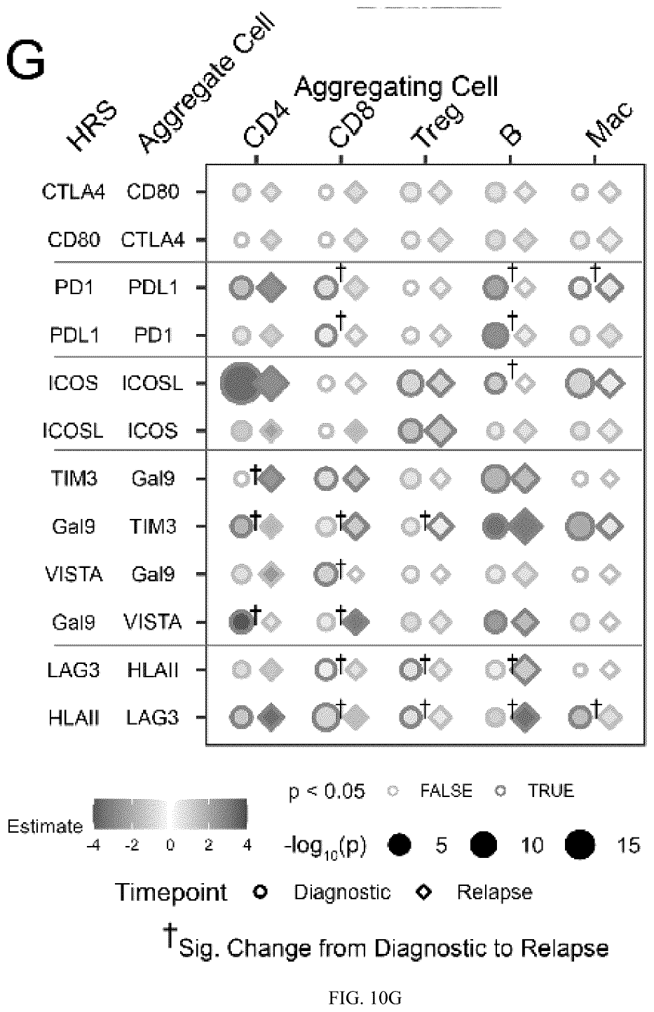

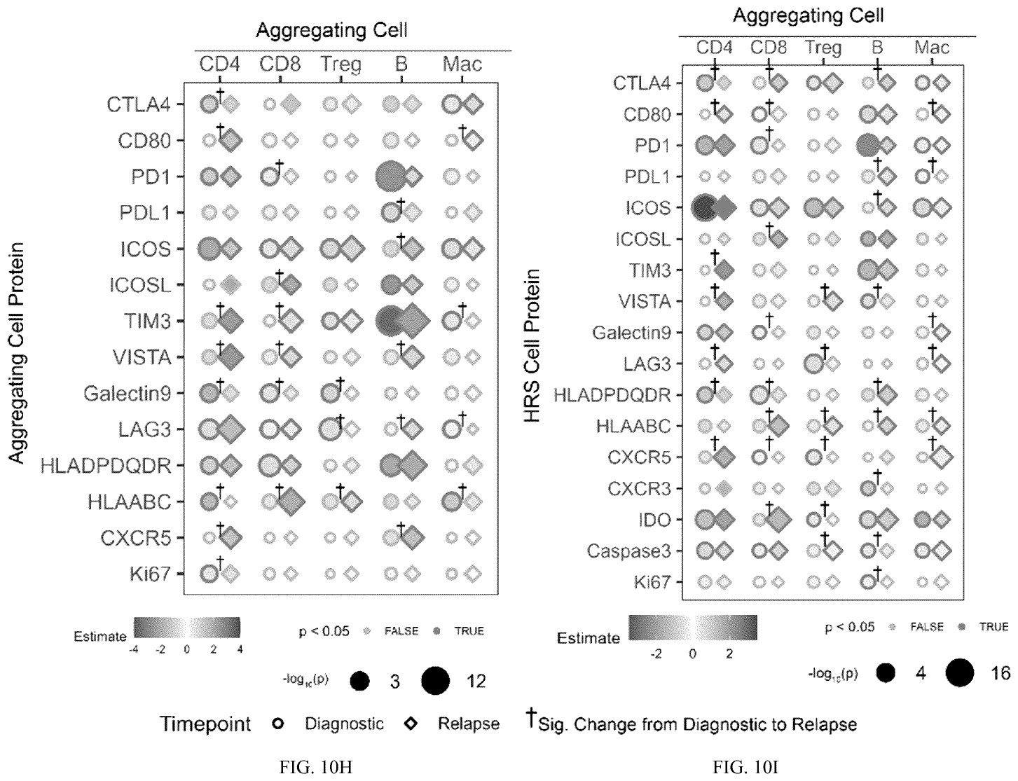

To quantify the organization of the heterogenous HL TME, we calculated spatial metrics from a single-cell perspective. Using a modified nearest neighbor (NN) analysis (FIG. 10A), we calculated spatial enrichment scores to describe every cell's spatial enrichment with the 10 major cell types. Homotypic interactions-cell interactions with other cells of the same type-were most common, especially among B cells (FIG. 3A). Beyond homotypic interactions, tumor cells interact more with CD4+ T, cDC, and myeloid cells, and less with B, CD8+ T, and endothelial cells. A correlation plot summarizes the significance of the spatial enrichments observed (FIG. 10B). The spatial enrichment scores were used for two purposes: 1) as input variables for biomarker candidates; and 2) to organize single cells into distinct local microenvironments (similar to “niches” or “neighborhoods”).

We used the spatial enrichment scores to isolate comparable regions of analyzed tissue between patients. Due to tissue heterogeneity and the relatively small size of each patient's analyzed tissue (˜1 mm²), a frequent concern in IMC analysis of TMAs is the generalizability of comparisons across patients in the face of heterogeneity. To mitigate this, we isolated cells in similar HRS cell-enriched microenvironments to perform a “digital biopsy” by selecting cells with a tumor spatial enrichment score >0.5 (tumor region) or a nearest-neighbor distance to HRS cells of <10 μm (tumor contact, FIG. 3B). Both types of tumor-enriched regions had increased HRS and CD4+ T cell proportions and reduced CD8+ T cell and B cell proportions. The primary difference between the two biopsies was their treatment of isolated tumor cells, where were ignored by the tumor region biopsy (purple dashed circle).

We also used the spatial enrichment scores to identify different types of local TME/niches using k-means clustering. Each cluster represents local regions of cells with enriched or depleted spatial interactions between specified other cell types. There was no well-defined elbow to determine the optimal cluster number, so a cluster number of 15 was used (FIG. 10C). The unique spatial architecture of HL is reflected in the composition of spatial clusters (FIG. 3C). HRS tumor cells were found in clusters enriched in CD4+/Treg cells (Cluster 1), T and B cells (Cluster 12), or myeloid cells (Cluster 14). Organized cell structures such as residual follicles (B cell rich clusters 13 and 11) were found along with B and T cell mixed zones (Clusters 1, 5, 7, 8, 9, 10, 11, 12). No tumor-only clusters were observed due to the low density of HRS cells. The composition of the niches led to niche-specific protein expression on major cell types (FIG. 3D). In lymphocyte-rich clusters, increased expression levels of HLA proteins were observed in HRS and dendritic cells (Clusters 6, 7, 9, 10, 12). Among macrophages, CD163 expression was negatively correlated with dendritic cell proximity (Clusters 1, 9, 10, 12 vs 2, 4, 6, 14, 15). Among clusters with significant HRS tumor presence (>5%), some HRS immune checkpoint proteins were correlated with proximity to B cells (CTLA4) and proximity to myeloid cells (PD1). We next focused on a specific spatial pattern example that was more localized and 5characteristic to HL.

Hyper-local CD4+ T cell clustering around HRS cells has been historically defined as a “rosette”. We hypothesized that cell aggregation around HRS cells, whether as CD4+ T cells or a different immune cell type, was the result of the local balance of inflammatory and suppressive signals, and furthermore, the extent of aggregation and functional state of aggregated cells may be linked to clinical outcomes. We first quantified cell aggregation by counting the number of cells of selected subtypes (CD4+, CD8+, Treg, Macrophage, B, FIG. 3E, FIG. 10D) in immediate proximity (<15 μm) of HRS cells. We found that aggregates formed with higher frequency than random chance in T cells and macrophages using a random replacement model (FIG. 10E-10F). Since aggregation was non-random, we sought to determine if it was dependent on functional cellular interactions.

We hypothesized that correlating the expression of receptor-ligand pairs on HRS and aggregating cells would help us understand how rosettes were formed and maintained. We measured the average cell-type specific protein expression on the aggregating cells and the central HRS cell as a function of the number of aggregating cells (FIG. 3F). By modeling the contributions of all positive receptor-ligand signaling pairs expression levels to aggregate size, we identified protein pairs with both positive (FIG. 3G, HRS-ICOSL/Treg-ICOS) and negative (HRS-HLADPDQDR/CD4-LAG3) correlations to aggregate size in diagnostic samples. Here, a significant correlation indicated that co-expression increased/decreased with increased aggregate size. These patterns provide evidence for a mechanism of positive/negative feedback in local microenvironments based on spatial cell arrangement.

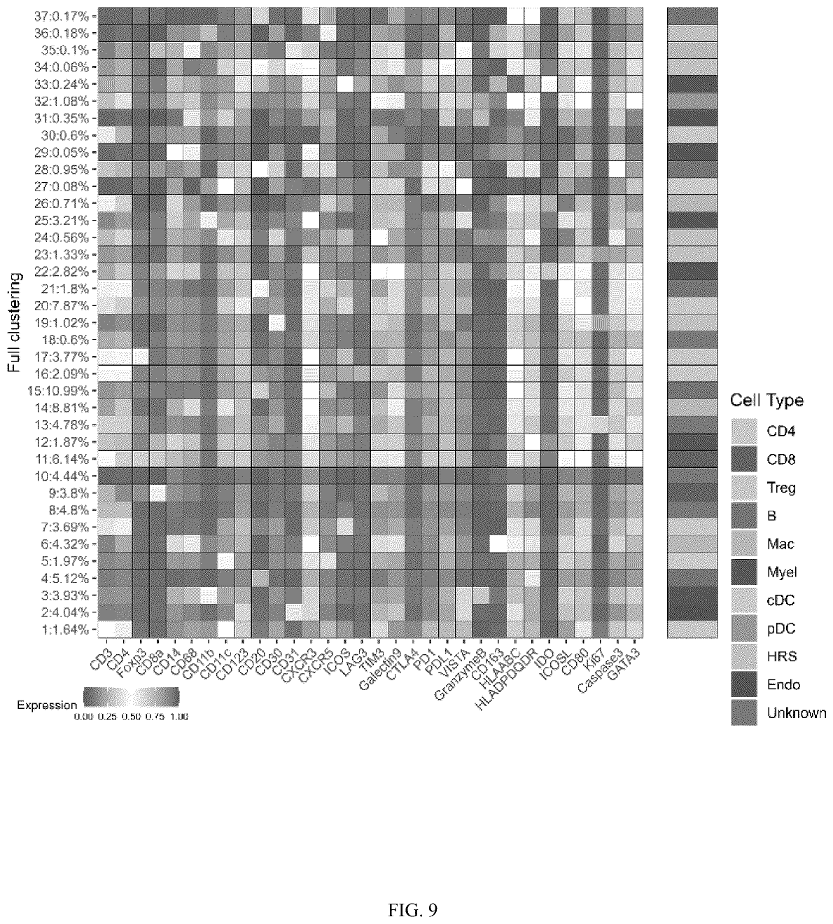

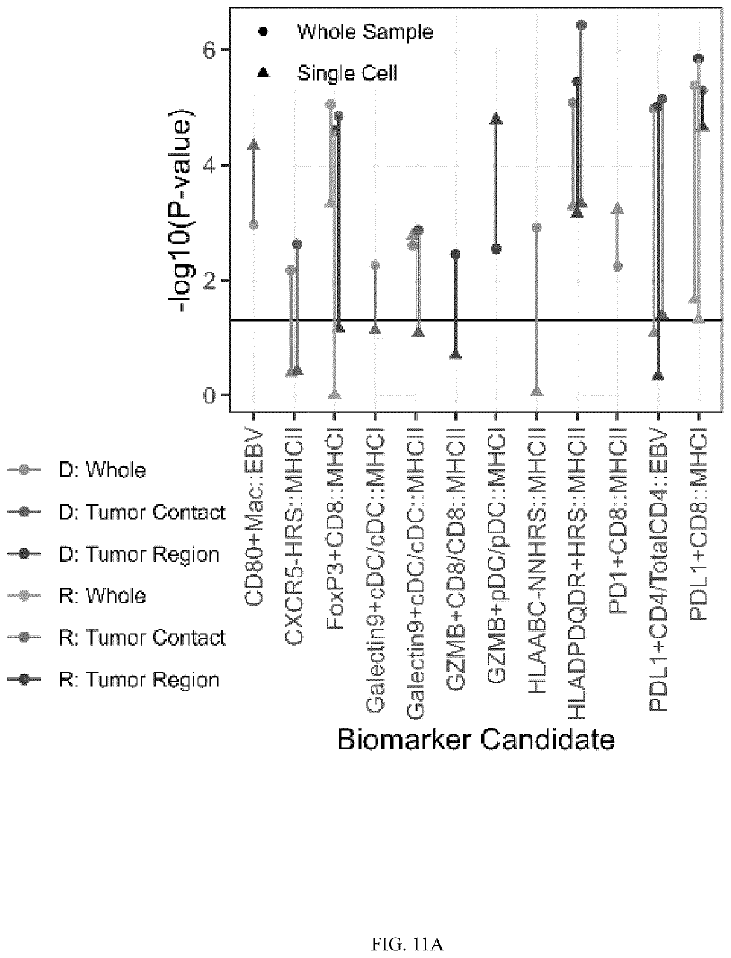

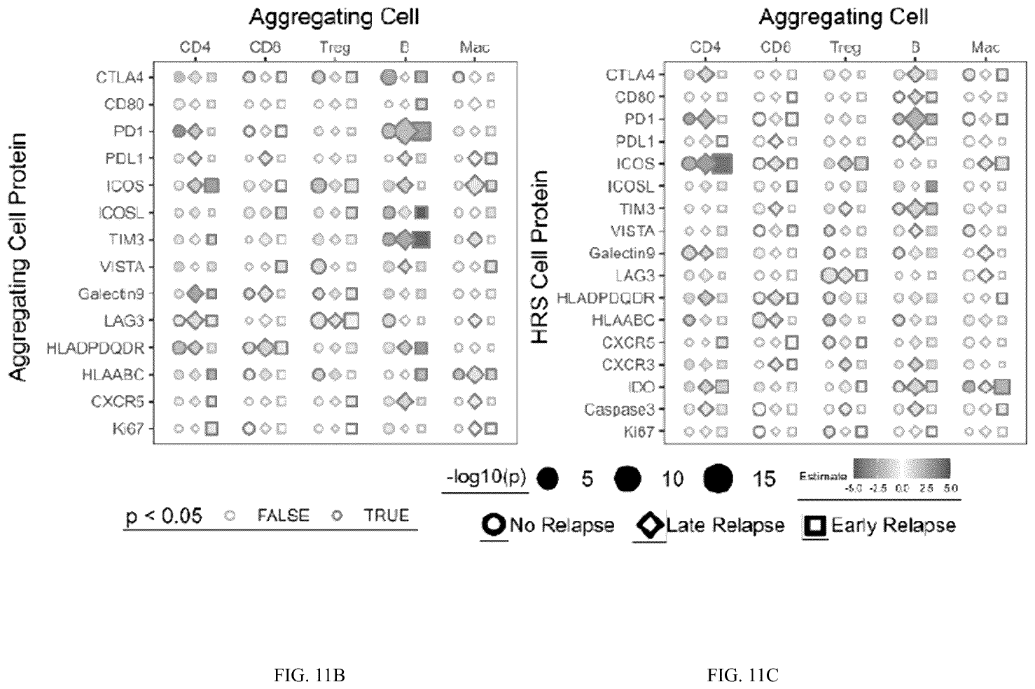

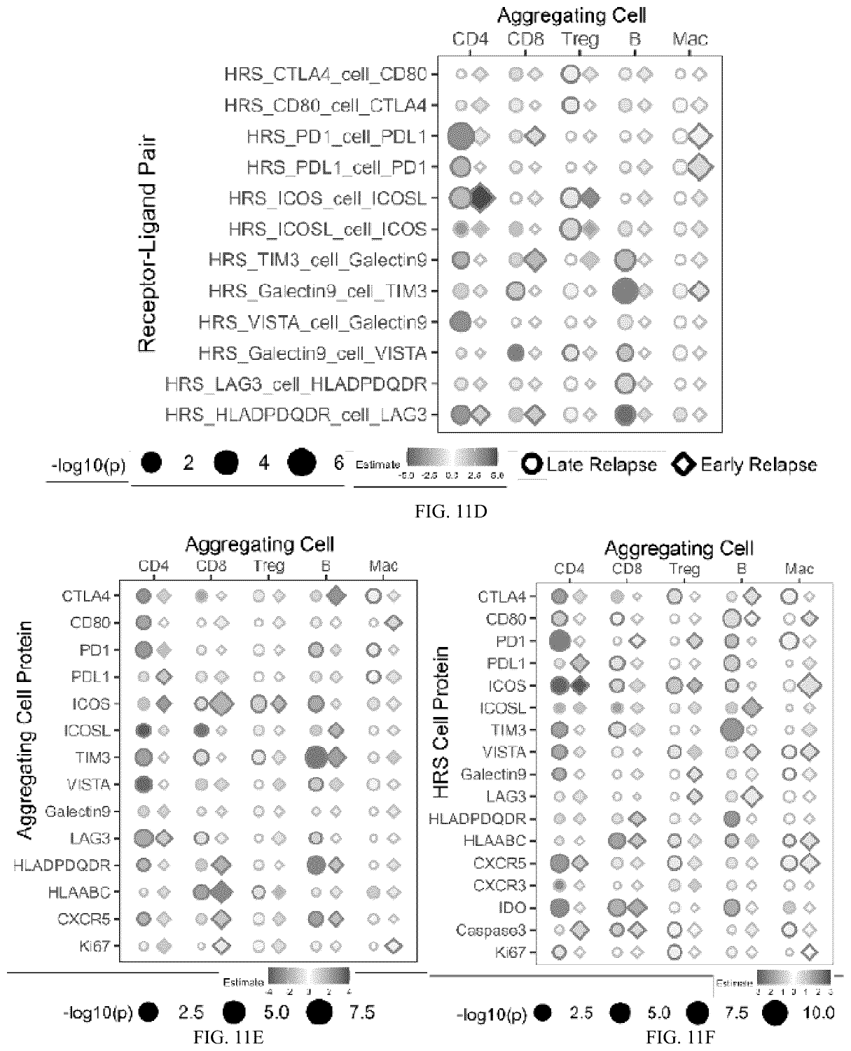

To examine if these protein expression and aggregation patterns had clinical significance we compared pretreatment diagnostic and posttreatment relapsed samples. Certain ligand-receptor pairs, such as HRS-ICOSL/Treg-ICOS and HRS-HLADPDQDR/CD8-LAG3 (FIG. 10G) were found across both diagnostic and relapse samples, and could represent universal aspects of HL biology independent of chemotherapy treatment. We observe significant reorganization in local PD1/PDL1, HLADPDQDR/LAG3, and Galectin9 signaling between diagnostic and relapse HL tumor biology (FIG. 3H-31). Galectin9 was differentially associated with aggregation between diagnostic and relapse samples in many instances, including on CD4+ T cells (FIG. 3H). PD1/PDL1 signaling increased with CD8+ T cell and B cell aggregates at diagnosis but not relapse. We observe significant PD1/PDL1 signaling with macrophage aggregation at relapse but not diagnosis in this hyper-local setting. HLADPDQDR/LAG3 was negatively associated with CD8+ and Treg aggregates in diagnostic samples only, before emerging as a significant association in B cell aggregates upon relapse. These ligand-receptor results, along with HRS protein-only or aggregating cell-only results (FIG. 10H-10I) show the evolution of local environments of HRS tumor cells between diagnosis and relapse and the pathways potentially shaping these environments.

### Biomarker Validation Using IMC

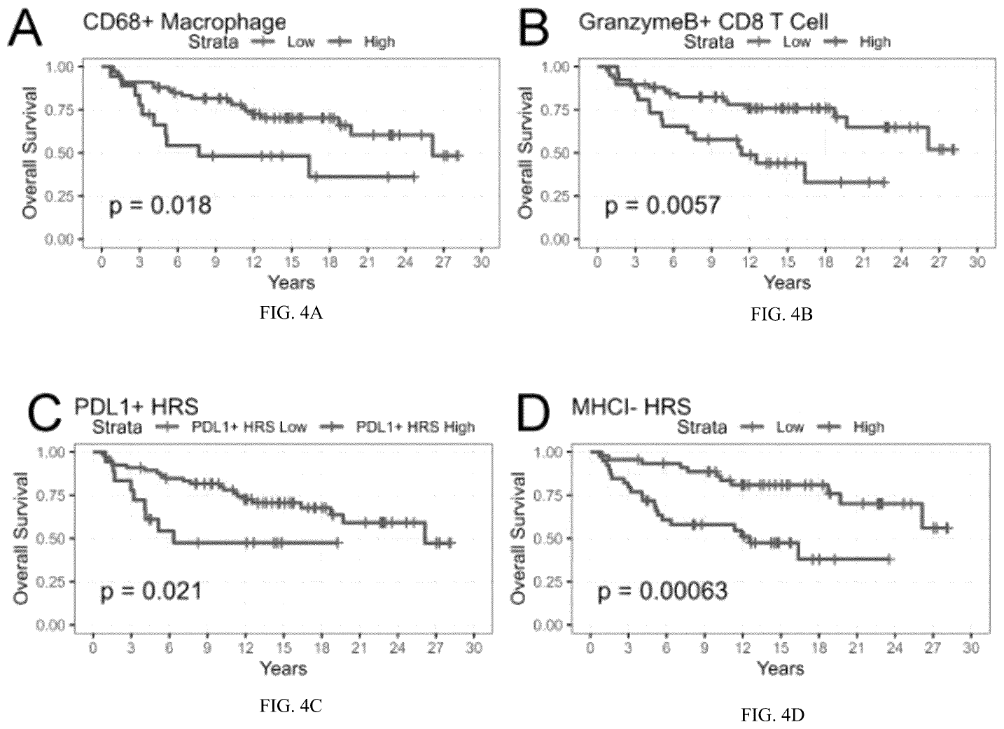

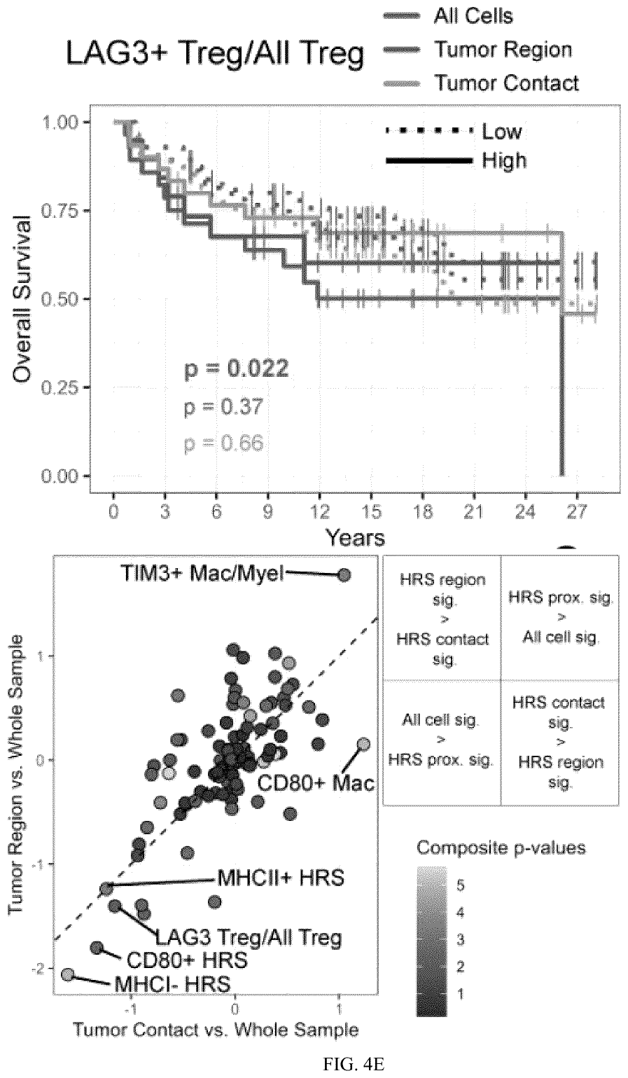

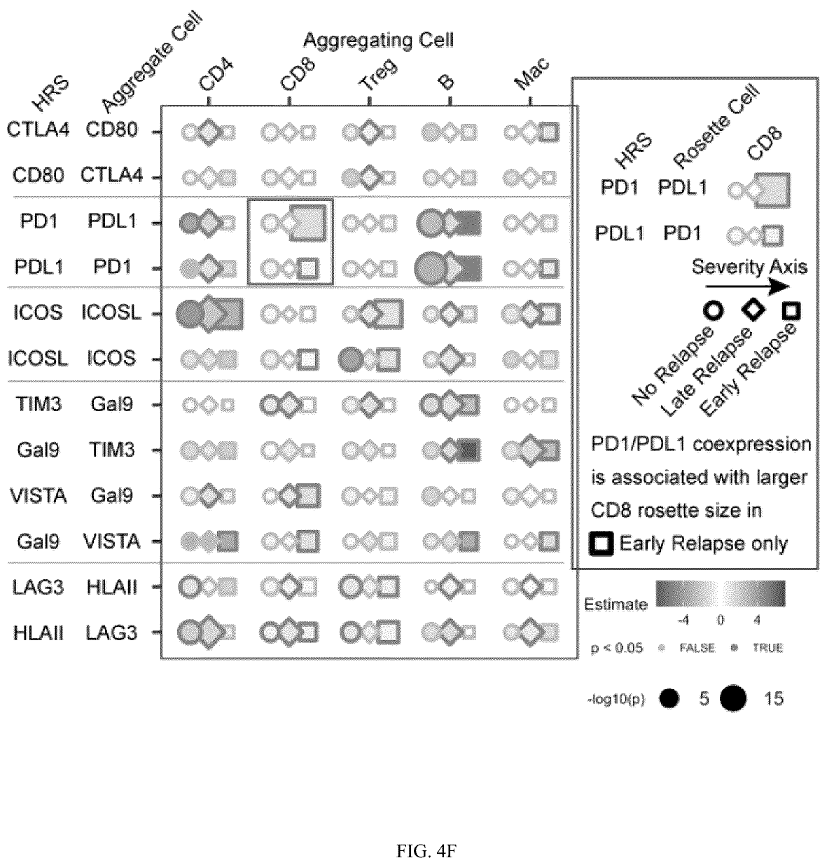

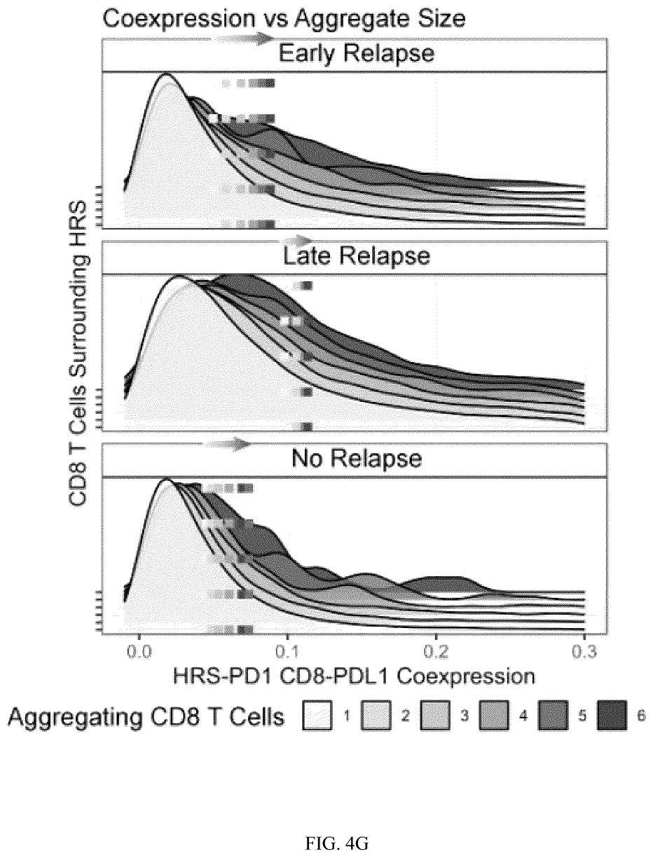

Although most patients with HL are cured with initial chemotherapy, for the subset of patients who are refractory or experience early relapse the overall prognosis is much poorer as they are less likely to be cured with salvage therapies like autologous bone marrow transplant. To better identify biomarkers that could identify these high-risk patients, we focused our analysis on diagnostic samples associated with early, late, or no relapse. We used IMC analysis of our cohort to identify and validate protein and cell patterns linked to survival and patient outcomes. We called these measured cell percentages or expression levels “biomarker candidates” and determined their significance using overall survival as the clinical endpoint initially. First, biomarkers from literature were compared to equivalent IMC biomarker candidates. Biomarkers using sample-level proportions of cells such as CD68+ macrophages were readily validated with IMC (FIG. 4A). More granular biomarkers based on cell-specific expression, such as GranzymeB+CD8+ T cells (FIG. 4B) and PDL1+ HRS cells (FIG. 4C), were also validated. Immunohistochemistry-based biomarkers often use complex scoring strategies. Roemer et al proposed one such scoring strategy based on MHCI/II expression on the tumor cell relative to its neighbor cells. We used aggregation analysis to automate the identification and stratification of MHC-I-negative cells and validated this biomarker (FIG. 4D).

We hypothesized that spatial organization may be predictive of HL outcomes, and that it can be used to add additional context to biomarkers. Significant changes in cell composition were observed between patients based on their relapse status (no relapse, late relapse, early relapse) and sample timepoint (diagnostic, relapse). CD8+ T cell and B cell aggregates increased in late relapse patients, especially in their relapse samples. Among spatial niches, a myeloid cluster was significantly more abundant in non relapse patients while HRS-enriched spatial niches were enriched in relapse patients.

We used digital biopsies to compare tumor-enriched regions and tumor-contacting cells between patients to refine biomarkers of overall survival in diagnostic samples to be less sensitive to intrapatient heterogeneity. LAG3+ Tregs as a percentage of total Tregs were one such biomarker that was predictive only when all cells were used but not in either digital biopsy, thus indicating that subtype of Tregs distant from the tumor were the predictive elements (FIG. 4D). On the other hand, CD80+ Macrophages were one of many biomarkers that became significant only in tumor-enriched regions (FIG. 4E).

Macrophage and HRS cell subtypes were the most frequently significant biomarkers along with established biomarkers such as GranzymeB+CD8+ T cells. This remained true when additional clinical outcomes such as disease-specific survival, 1st failure-free survival, 2nd failure-free survival, and postBMT failure-free survival were considered. Biomarker analysis for categorical clinical variables (EBV, MHCI, MHCII, Early Relapse, No Relapse) showed that CD8+ T cells and HRS subtypes were the most significant biomarker candidates, and HRS candidates were more spatially dependent. We also evaluated single cell variants for significant biomarker candidates (p<0.05 after Benjamini-Hochberg). For such subtypes, i.e. PD-L1+CD8+ T cells, we tested the equivalent cell-type-specific protein expression (PD-L1 expression on single CD8+ T cells). Most candidates were significant at both sample and single cell levels except for those tracking negative expression (HLAABC-HRS, CXCR5-HRS) or subtype percentages (GranzymeB+CD8+ T cell vs total CD8+ T cell).

We found ligand-receptor protein expression in local HRS immune aggregates to be dependent on the relapse status of patients (FIG. 4F). For example, while HRS-PD1/CD8-PDL1 signaling was significantly associated with CD8+ T cell aggregate size across all diagnostic samples (FIG. 3I), we found this to be significant in early relapse diagnostic samples only after stratifying diagnostic samples by patient relapse status (FIG. 4G). In contrast, PD1/PDL1 in CD4+ T cell aggregates was significant in non-relapse or late relapse samples only. Other early relapse patterns included HRS-ICOSL/Mac-ICOS, HRS-Galectin9 and TIM3 on B cells and Macrophages, and HRS-Galectin9 and VISTA on CD4+, CD8+, B cells, and macrophages. Significant patterns in non relapse patients included HRS-TIM3/CD8-Galectin9, and LAG3/HLADPDQDR signaling on CD4+ T cells. Finally, some patterns were selectively significant in late-relapse patients only, including CTLA4/CD80, ICOS/ICOSL expression on B cells, and LAG3/HLADPDQDR on B cells and macrophages. These patterns may reflect the ability of immune cells to regulate HRS behavior and feedback mechanisms of signaling that result in increased immune recruitment. Furthermore, these patterns show that a ligand-receptor signaling pathway may have different consequences for tumor relapse status depending on the immune cell type involved.

### Hodgkin Lymphoma Biomarker Discovery

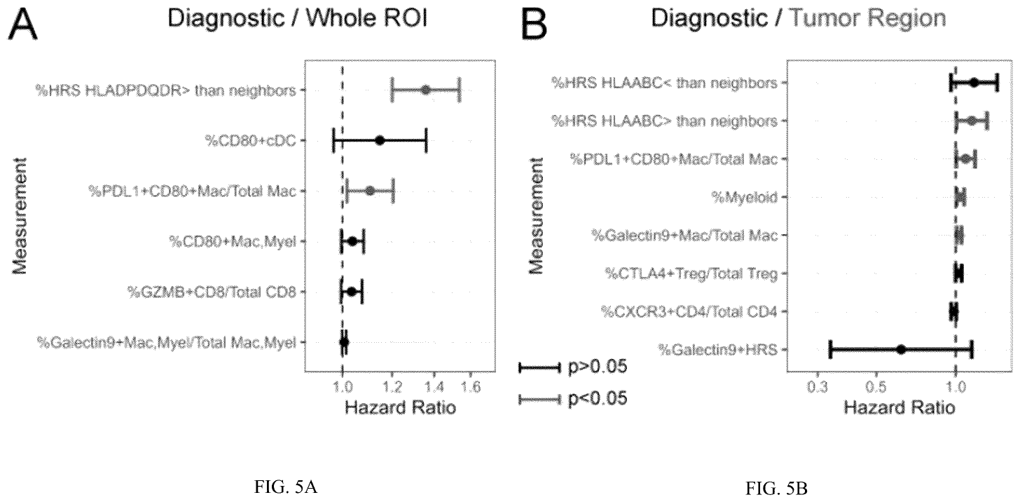

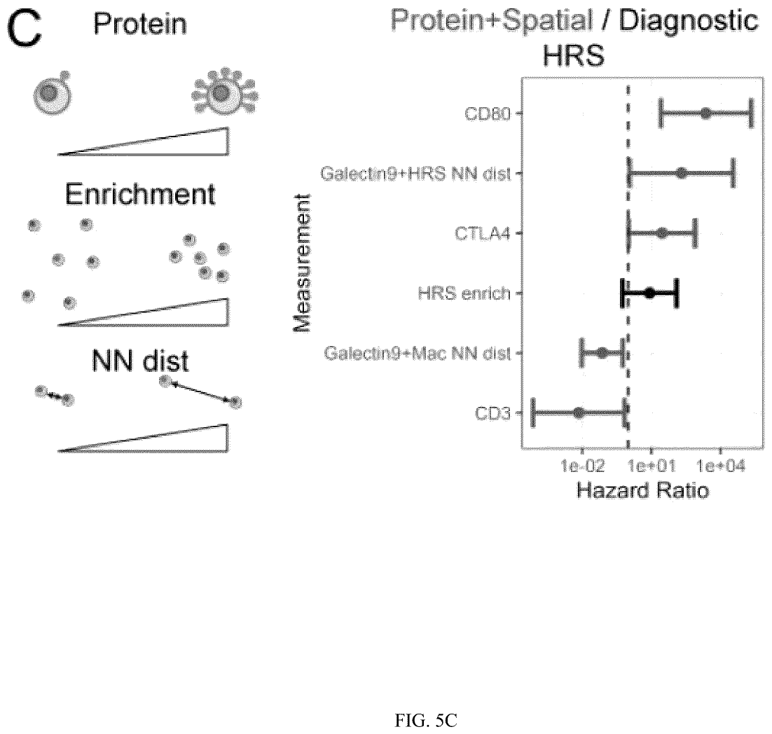

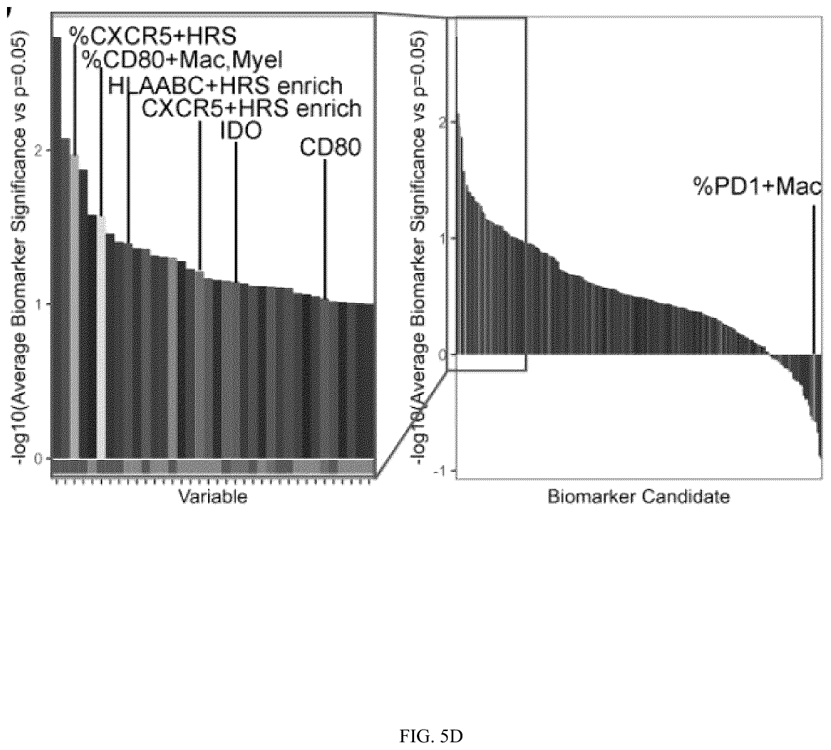

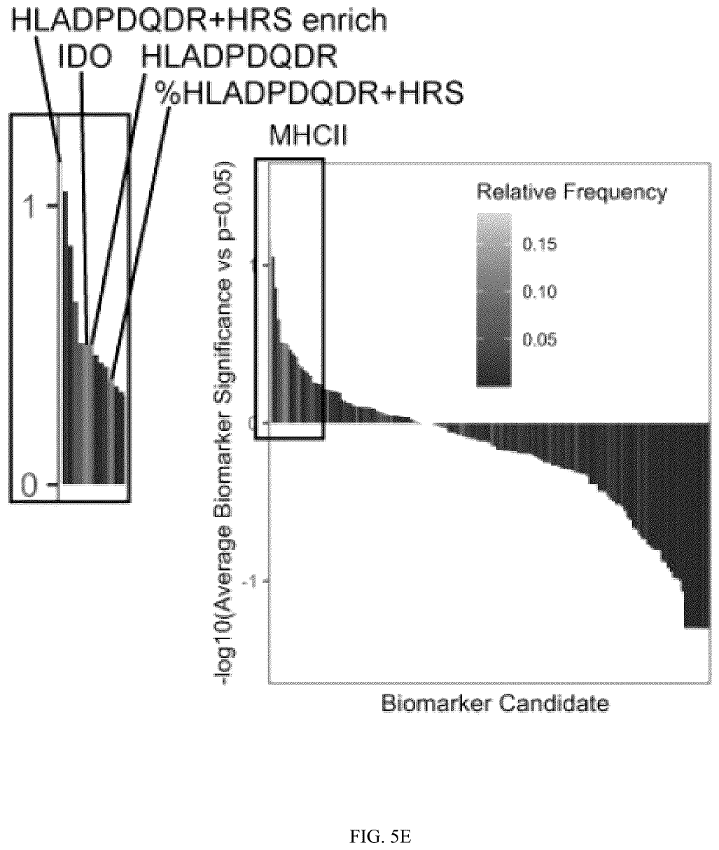

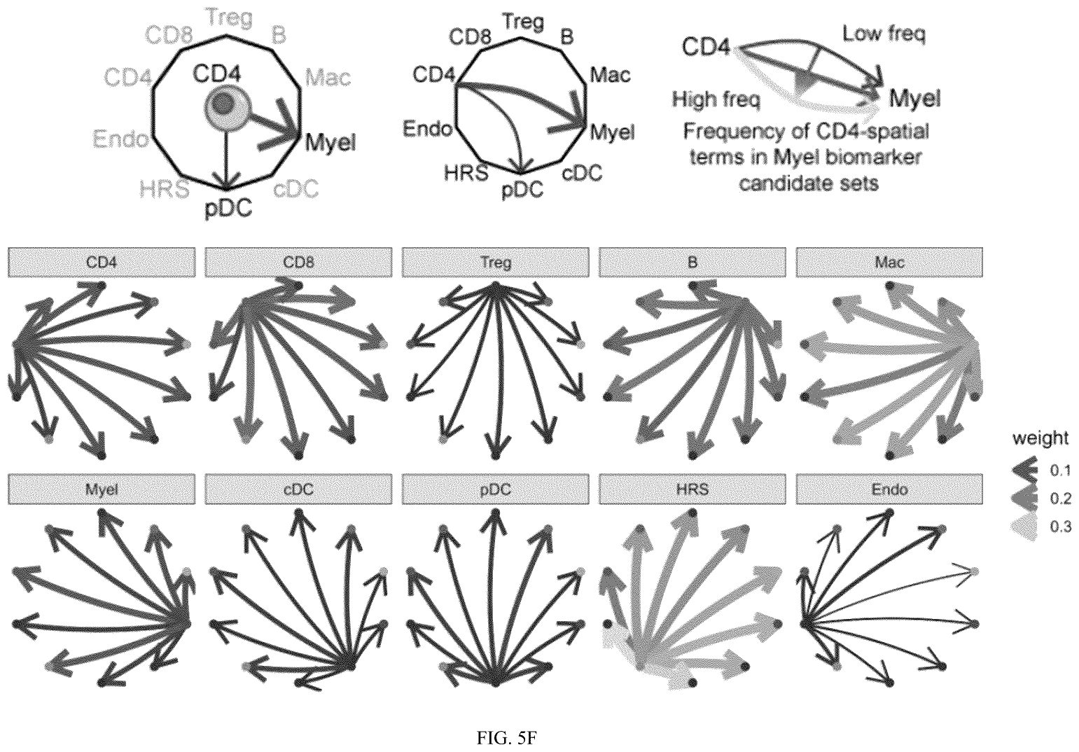

The biomarkers explored above were focused on cell-type-specific expression, which can involve as many as 3-5 proteins in the context of a particular cell type. We searched for new combinations of sample-level cell proportions that predicted overall survival using a dimensional reduction and variable selection strategy (LASSO_plus) based on the least absolute shrinkage and selection operator (LASSO), single variable selection, and stepwise variable selection. In diagnostic samples, LASSO_plus selected HLADPDQDR HRS cells with higher expression than their neighbors and PDL1+CD80+ Macrophages as significant biomarker candidates, with GranzymeB+CD8+ T cells, Galectin9+ Macrophages and Myeloid cells, and other CD80+ cells as marginal candidates (FIG. 5A). The LASSO_plus strategy allows for a comprehensive search for broader potential biomarkers without the need for manual intervention and facilitates the construction of a model based on selected variables. This is achieved through the utilization of our automated R function, LASSO_plus, which is accessible in our new R package csmpv (github.com/ajiangsfu/csmpv).

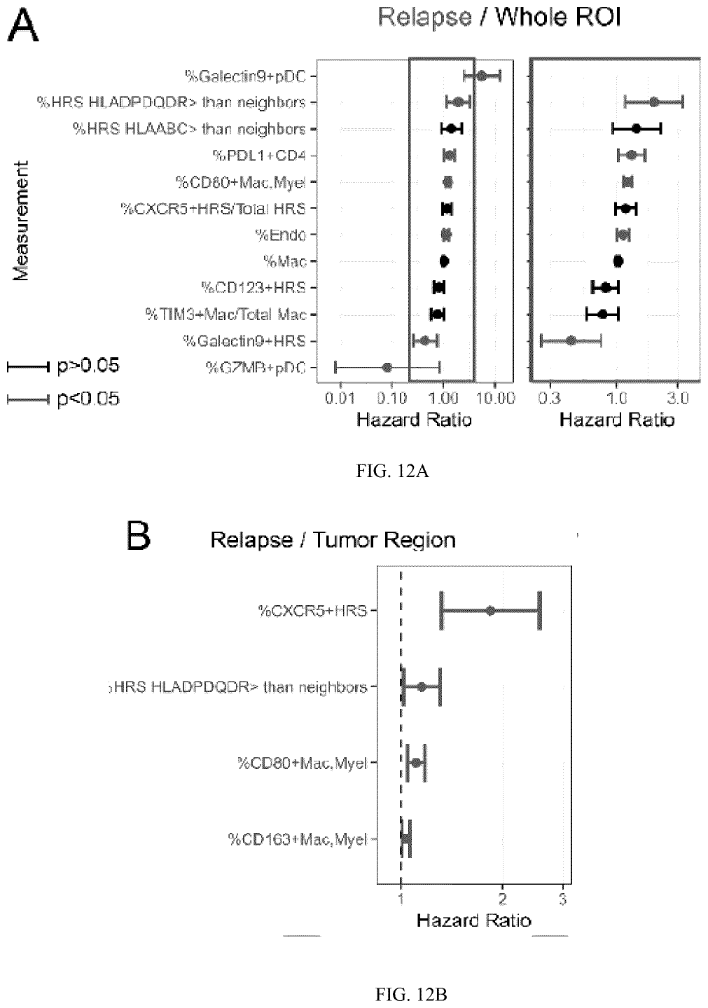

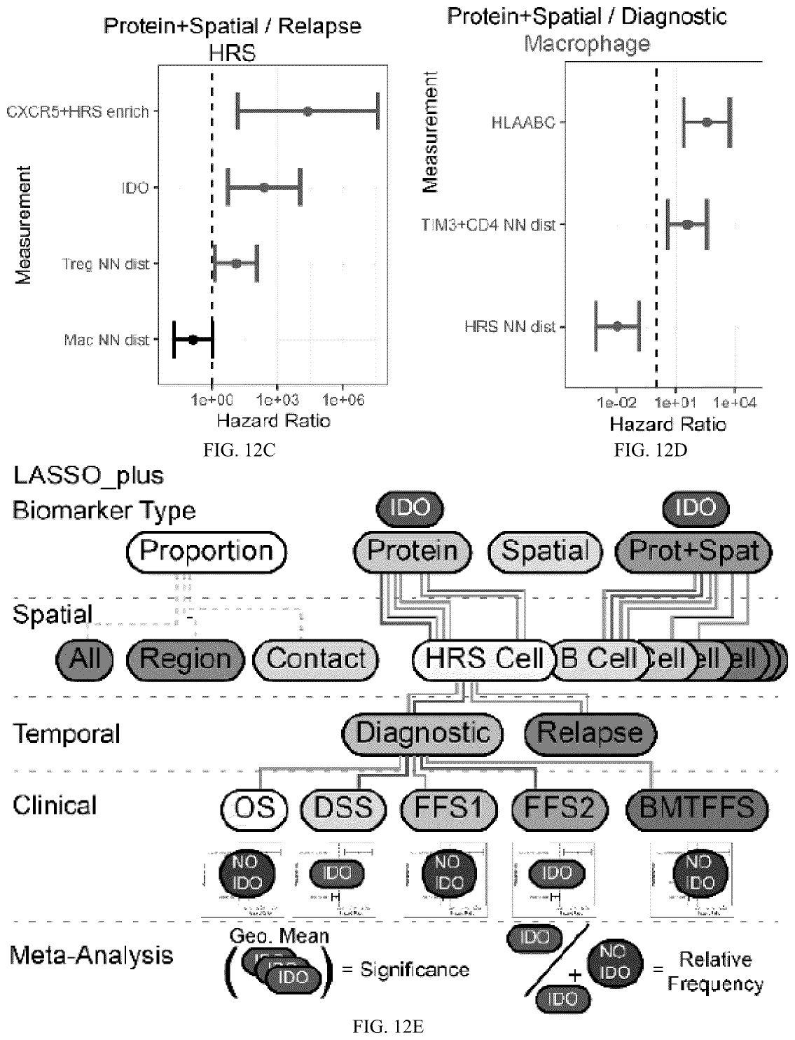

Our spatial and clinical data allowed for additional biomarker candidate testing using LASSO_plus. Using the digital biopsy to isolate tumor regions revealed a different set of biomarker candidates in diagnostic samples, including HLAABC-HRS cells and Myeloid cells (FIG. 5B). Relapse samples reflect an altered tumor microenvironment, which yielded different biomarker candidates, including Galectin9+ HRS and pDCs, PDL1+CD4+ T cells, and HLADPDQDR+ HRS cells (FIG. 12A-12B). Cell specific average protein expression and spatial enrichments and distances were explored as a cell-centric biomarker format using LASSO_plus instead of an image-level proportion biomarker in diagnostic and relapse samples (FIG. 5C, FIG. 12C). These two biomarker formats are related (see Galectin9+ macrophage significance in both cases), but the cell centric biomarker may be less sensitive to changes in cell proportions caused by heterogeneous sampling. This pipeline can also be used to explore biomarkers measured on other cell types such as macrophages (FIG. 12D).

We searched for proteins, spatial metrics, and cell proportions with the highest average significance (geometric mean vs. p=0.05), and highest relative frequency among all LASSO_plus biomarker candidate sets and plotted them as waterfall-style plots (FIG. 5D, FIG. 12E). Among proteins, IDO, Galectin9, CD3, and CD80 were an order of magnitude more significant than p=0.05 on average (geometric mean of p<0.005), while IDO, CD80, and CD3 (along with CXCR5, CXCR3, and CD20) appeared in more than 350 sets (>2% of all possible). Spatial enrichment of Tregs, HLAABC+ HRS, CD163+ macrophages, PDL1+CD4+ and HRS cells, and Galectin9+ Macrophages were the most significant biomarker candidates while HRS, B cells, CXCR5+ HRS and B cells, HLAABC+ HRS cells, and macrophages were the most frequent.

A similar biomarker candidate discovery analysis for clinical factors (EBV status, MHCII status, No Relapse, Early Relapse) revealed proteins and spatial metrics biomarker candidates for these clinical factors (FIG. 5E). MHCII status was strongly associated with spatial proximity to HLADPDQDR HRS tumor cells, HLADPDQDR expression, and HLADPDQDR+ HRS cells as expected (FIG. 5L). Comparing spatial measurements only and their relative significance to specific cell types (FIG. 5J), Macrophage and HRS spatial measurements were most consistently significant in relation to other cell types, while Endothelial, Treg, and cDC spatial measurements were rarely significant.

### Discussion:

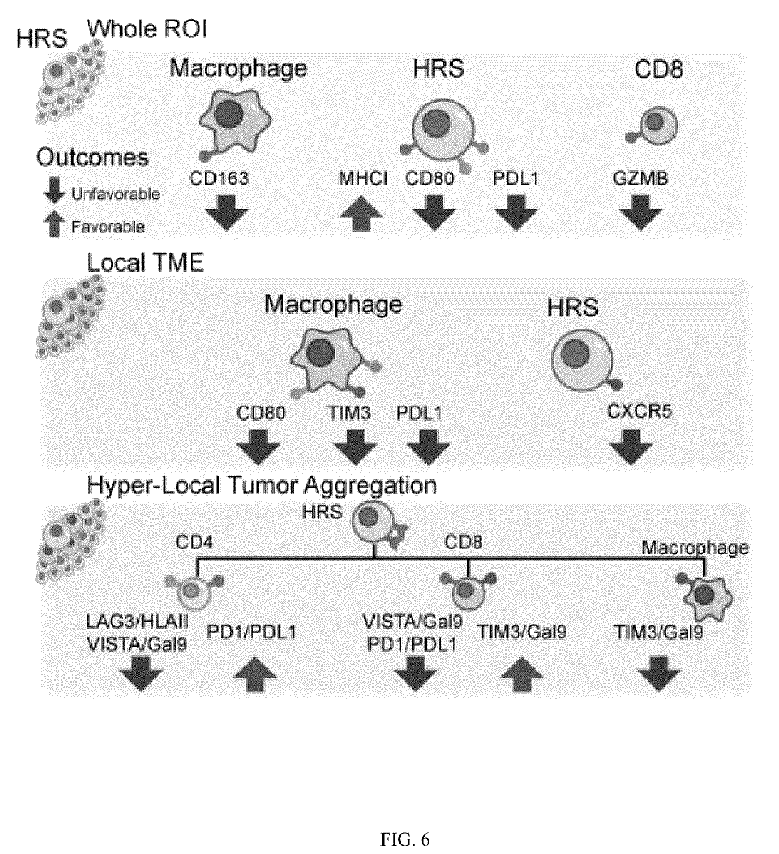

This spatial protein study of matched diagnostic and relapse samples of relapsed/refractory Hodgkin Lymphoma is a resource with analysis tailored towards biomarker discovery. We identified expression patterns that appear significant to predicting outcomes in HL across different intercellular distances (FIG. 6). In the unselected regions, CD163+ macrophages, GranzymeB+CD8+ T cells, and various HRS subtypes were significant predictors, as previously reported. In local microenvironment niches, significant patterns were observed in CD80, TIM3, and PDL1 on macrophages, and CXCR583 on HRS cells. At the hyper-local cell contact level, ligand-receptor interactions appear to drive aggregate formation, involving known (LAG3/HLAII with CD4+ T cells, PD1/PDL1 with CD4+ and CD8+ T cells, TIM3/Galectin9 with CD8+ T cells and macrophages) and proposed (VISTA/Galectin9 with CD4+ and CD8+ T cell) signaling pairs. Alongside these promising biomarker candidates, our analysis also generates negative results. For instance, the study that proposed local expression of HLA-ABC's usefulness as a biomarker (FIG. 4D) also proposed that local expression of HLA-DPDQDR would not be significant, which we also corroborated (p=0.24).

We propose that the hyper-local aggregate biomarkers have 3 primary advantages. First, they incorporate both tumor and immune signaling information. Second, they are calculated within samples and may therefore be less sensitive to technical issues such as batch effects or heterogeneous tissue sampling. Third, they are spatially restricted and not overreaching. We can propose a simple hypothesis for aggregate biomarkers, where upregulated checkpoint expression between tumor cells and their immediate neighbors is more indicative of checkpoint signaling between tumor and specific immune cell types than other non-spatial measurements such as global expression of immune checkpoints or immune abundance. While larger spatial niches involving tens to hundreds of cells were also found with statistically significant survival associations in our analysis, they are more descriptive in nature and are too complex to hint a mechanistic basis for their formation.

Spatial analytics is undergoing a modern transformation by the introduction of highly multiplexed technologies with single cell-resolution. Clinical use of multiplexed IHC of up to 7 markers is becoming standardized (CLIA approved). Further multiplexing to 100+ marker analysis possible using cyclic imagers. There are several distinctions between this pipeline and others. First, we used single cell spatial distance metrics to perform spatial and microenvironment analysis, instead of community- or graph-based strategies. We also added biomarker candidate discovery and validation tools here to address the gap between spatial IMC analysis and translation to clinical biomarkers. Unique to our study, we allowed cells to carry multiple labels of user-defined categories. Our approach was tailored towards clinical studies, which are often structured around single protein- or cell type-based hypotheses that can be lost or minimized by dimensional reduction. This design interfaced with our hypothesis testing and biomarker candidate validation tools, which guide the user towards the generation of standardized Kaplan-Meier survival curves and hazard ratios as well as spatially-informed biomarkers.

We proposed IMC as a tissue-efficient strategy for biomarker discovery and validation. We emphasized 4 previous biomarker studies (FIG. 4A-4D, CD68+ macrophages, GranzymeB+CD8+ T cells, PDL1+ HRS, MHCI-HRS, FIG. 4A-4D) that collectively represented 634 patients. Each study represents a significant investment in time and resources to validate, which was condensed into this single study using IMC. Our spatial strategy using digital biopsies also mitigated the problem of tumor heterogeneity causing biases in IMC-sized ROIs sampled from tissue. This strategy applies to all imaging studies, which will be important until the price of analyzing whole slides decreases. IMC can be applied broadly to identify parsimonious combinations of 3-5 proteins as biomarkers for clinical grade multi-color IHC platforms.

Findings from this study offer new insights into prognostic elements based on spatial patterns. Among the biomarker candidates generated by our analysis, we identified candidate proteins such as PDL1, TIM3, LAG3, and CXCR5 deserving of further attention and identified the specific cell types and contexts for potential biomarkers. For HL, the relatively high success rate of ABVD chemotherapy indicates that secondary outcomes, such as 1st or 2nd Failure Free Survival (FFS) and post-bone marrow transplant (BMT) FFS, may be more clinically significant. We performed a companion study to this one focused on translating IMC studies to a biomarker assay for postBMT FFS using IHC. In that study, we presented additional strategies to refine LASSO_plus variable selection, and demonstrated a protocol to select a parsimonious biomarker set of 6 proteins for IHC and standardize IMC and IHC data comparisons. We used the protocol to validate a new HL biomarker (RHL4S), based on the spatial arrangement of CXCR5+ HRS cells, PD1+CD4+ T cells, tumor-associated (CD68+) macrophages, and CXCR5+ B cells. See Example 8. With the coming of age of digital pathology and incorporation of multiplexed IHC into routine diagnostics, methods to identify these spatial patterns will become increasingly important for effective prognostication.

Spatial context expression requires interpretation, as in the aggregate studies we presented here. For instance, HRS cell checkpoint expression counterintuitively increased with CD4+ T cell aggregate size, especially in late relapses. Such HRS cells may lack other immunosuppressive mechanisms which then allows immune cells to be recruited to the HRS aggregates despite checkpoint expression. Non relapse, late relapse, and early relapse disease are also too complex to fall neatly in a continuum. One hypothesis is that late relapses, which occurred as late as 18 years after diagnosis, are due to failures in immune surveillance, which may manifest in our observed differences in aggregate composition and protein expression that are unique to late relapse patients, such as the increased frequency of CD8+ T cell aggregates (FIG. 4F-4G). By systematically quantifying local aggregate signaling we can validate spatial signaling insights. Cell segmentation of spatial data remains challenging especially in large cells or cells with concave or serpentine shapes in tissue (HRS, DCs). With imperfect segmentation and ambiguous cell phenotyping, human input is used to simplify data, usually by omitting problematic cells, or binarizing or simplifying the expression of phenotyping markers to remove ambiguity. In our study, we found that HRS-ICOS/CD4-ICOSL was observed as a highly significant association with CD4+ T cell aggregate size. However, ICOS is not believed to be expressed on HRS cells. Thus, “expression” of ICOS may be a technical observation associated with increased clustering of CD4+ T cells and imperfect segmentation, rather than biological HRS cell expression, which is reinforced by the universal significance of this protein expression pattern across all timepoints and patient outcomes. Commercial spatial transcriptomics now present an alternative spatial tool to spatial protein analysis, and the strengths and weaknesses of the technologies must be weighed. Currently, spatial transcriptomics is limited to the discovery setting due to its cost, and we believe the translational biomarker pipeline established here represents an essential use case for IMC. The amount of biomarker candidates generated by IMC is beyond what can be reasonably tested and validated using separate gold standard multi-color IHC experiments, the materials and reagents used in IMC can be rapidly translated to IHC in clinical settings, and the tools presented here can accelerate translational biomarker studies in a variety of diseases and clinical challenges.

### Sample Collection and TMA Assembly Details

The patient cohort was described in J Clin Oncol, 2017, 35, 3722-3733, which analyzed gene expression profiling of relapsed and refractory HL. We analyzed a tissue microarray of a cohort of 93 total HL patients treated at British Columbia Cancer comprising 73 diagnostic and relapsed paired samples and 20 primary biopsies of patients who were cured after ABVD treatment for a total of 256 ROIs with up to 2 ROIs per biopsies when available (average 1.76). Patients were selected according to the following criteria: patients received first-line treatment with doxorubicin, bleomycin, vinblastine, and dacarbazine (ABVD) or ABVD-equivalent therapy with curative intent; patients experienced CHL progression/relapse after primary (refractory disease or relapse); and tissue derived from an excisional biopsy was available. This study was reviewed and approved by the University of British Columbia-BC Cancer Agency Research Ethics Board (H14-02304), in accordance with the Declaration of Helsinki. We obtained written informed consent from the patients or informed consent was waived for the samples used in this retrospective study.

### Antibody Conjugations

Antibodies were purchased from Fluidigm Inc., now Standard Biotools, in conjugated form. Antibodies that were unavailable were conjugated using MaxPar kits. TMA slides were dewaxed in 3 washes of xylene and rehydrated by successive washes in 100%, 95%, 80%, and 70% ethanol in distilled water. After washing with the alcohol gradient, the slides were immersed in Tris-EDTA antigen retrieval solution for 30 minutes at 95° C. and were left to cool down inside the solution for 30 minutes more at room temperature. After the antigen retrieval step the slides were blocked with 3% BSA for 45 minutes and then were stained overnight with the antibody panel at 4° C. The next day the slides were washed twice with PBS-0.1% Triton X solution and 1×PBS for 8 minutes each. The slides were then incubated with 191 Iridium, a nuclear stain, for 40 minutes. After that, the slides were washed with dH2O and dried off before ablation. The slides were ablated using the Hyperion/Helios Imaging Mass Cytometry platform (Standard Biotools) at a rate of 200 H, and images were acquired for analysis.

### Image Analysis

Raw image files were generated using MCD Viewer (Standard Biotools). Segmentation was performed using the IMC Segmentation Pipeline (Bodenmiller lab) with modification. First, a subset of antibodies was chosen for Ilastik training. We define groups of membrane-specific (CD14, HLA-ABC, CD68, CD31, CD4, CD20, CD8a, CD30, CD3, CD45RO, HLA-DPDQDR) and nuclear-specific markers (Ir191, Ir193) and add 4 additional channels to the Ilastik training: the sum of all antibody channels, the sum of nuclear-specific channels, the sum-of membrane-specific channels, and a Sobel-filtered nuclear channel which highlights the edges of nuclei by emphasizing sharp changes and de-emphasizing constant regions of nuclear signal. We did not quantify the effect of adding these channels as each Ilastik training is a highly specific and unique series of drawn regions for every image.

Segmented cell data was processed using Python and R. Cells smaller than 5 μm² or larger than 300 μm² were removed from analysis, and ROIs with fewer than 500 cells were removed from analysis. Particular challenges in segmentation of IMC data included some multi-nucleated cells, as well as cell fragments due to the nature of tissue slices. HRS and DC cells were likely most affected by these limitations. Mean cell protein values were transformed by hyperbolic arcsin, scaled, censored at the top 1%, and all clustering steps were performed using Rphenograph with k=15. Automated clustering was performed by calculating median protein values of protein expression, normalizing to z-scores, and gating clusters. Cell type phenotype labeling and triage for reclustering was performed manually at the cluster level. Cells were allowed to carry multiple phenotype labels.

To perform quality control on phenotyping, we measure the heterogeneity of cell prevalence across different measurement units (i.e. patients) using the Shannon informational entropy (FIG. 8B). For any cell type, entropy is minimized when all cells are found in a single patient, and it is maximized when every patient has the same proportion of that cell type. While we do not expect every cell to be found at the same frequency in each patient (maximum entropy), very low entropy can be indicative of batch- or patient-specific-effects.

Our script presents two strategies for phenotyping depending on the size of the data set: full clustering (FIG. 8A) or metaclustering. Beginning with the first tier of cell phenotyping markers, full clustering uses a single clustering step for every cell, which can be computationally intensive as data sets grow beyond 1 million cells and more than 10 proteins are used. Metaclustering (used in this study) first performs clustering at a smaller subset level (image-level, patient-level), then performs clustering of mean expression levels of clusters. Automatic labels were assigned by calculating z-scores of protein expression for clusters and labeling each cluster with all cell types based on protein expression.

Within each of these automatically labeled clusters, we performed a second Phenograph step and manually sorted each cluster into the cell type of interest. Remaining cells unable to be classified were subjected to a final clustering step and z-scores of cell expression were used to assign cell types, with multiple cell type assignments per cell allowed. After manual curation, each major phenotype underwent a Phenograph step using cell-type-specific markers and relevant phenotype subtypes of interest were defined. UMAPs were generated using the uwot package for all cells as well as cell subtypes using default settings.

### Spatial Analysis

For spatial analysis, the distance of each cell to its 5 nearest neighbors of every cell phenotype label was calculated, capped at 50 μm, scaled from 0-1, and inverted. These distance metrics describe the local enrichment for a specific cell type to each cell, and were used in clustering using k-means clustering (k=15 or 35) to define spatial niches/local microenvironments. Local aggregation-dependent protein expression was obtained by selecting aggregating cell types (CD4+T, CD8+ T, Treg, macrophage, B cell), and identifying all cells of the aggregating cell type within the contact radius (15 μm) of every cell. The number of aggregating cells within the radius and the mean protein expression were recorded. Ligand receptor expression was defined as the product of the ligand and receptor on the central cell and aggregating cell average, respectively. Aggregate-specific protein expression was calculated by constructing a generalized linear model of aggregate size as a function of aggregating cell protein, HRS cell protein, or ligand-receptor expression, and extracting p values for each of the biomarker candidates.

### Aggregate Substitution Test

Each ROI was independently analyzed for random replacement testing of aggregation frequency. For each type of aggregating cell, phenotype labels were reshuffled on every cell to match the original cell proportions, and the number of aggregates around HRS cells was calculated (defined by at least 3 aggregating cells). This process was performed 10,000 times, and the proportion of times for which the reshuffled image contained more aggregates than the observed aggregates was defined as the test fraction.

### Biomarker Analysis

For phenotype biomarkers, each ROI was summarized by the percent composition of every labeled phenotype. We used the mclust R package at its default settings to perform model-based clustering to stratify patients. Stratifications that produced greater than a 90:10 split were removed from analysis. Cox proportional hazards survival analysis was performed using the survival package in R. Biomarker analysis was performed on subsets of the data separated by temporal conditions (diagnostic/relapse, 2 conditions) and spatial conditions (whole image, tumor enrichment—cells with HRS enrichment >0.5, and tumor contact—cells with HRS nearest neighbor distance <10 μm), which we defined as “digital biopsies” (3 conditions). A total of 5 possible event codes (overall survival, disease-specific survival, freedom from first failure survival, freedom from second failure survival, and bone marrow transplant freedom from failure survival) were available, resulting in 30 possible survival analyses across the temporal and spatial conditions. Each biomarker candidate that was significant in at least 4 conditions was retained, and p-values were adjusted by the Benjamini-Hochberg false discovery rate correction within these retained candidates.

A linear or generalized linear model was used to analyze single-cell IMC data for continuous or categorical outcomes. The sample was considered as a clustering variable, and cluster-robust standard errors were computed. The analysis was conducted using the ‘stats’ package in R. Cox regression is readily used with patient-level biomarkers, and for single-cell biomarkers, Cox regression can be performed with a cluster or frailty variable, mixed models and generalized estimating equations, or by down sampling each patient to obtain equal numbers of cells to ensure that each patient is weighted properly. These strategies have not performed well in the IMC setting, and we recommend converting cell-level biomarkers into patient-level biomarkers to perform Cox regression as done here using the mean.

### LASSO_plus

Using sample-level data, we present a pipeline using LASSO_plus for variable selection, which is available in the R package “csmpv” (github.com/ajiangsfu/csmpv). This variable selection pipeline is processed with three steps using either cell proportions, mean cell-type-specific protein expression, or mean-cell-type-specific spatial metrics as variables. The 1st step is a customized version of LASSO. Variable selection is performed with the glmnet R package to extract candidate biomarkers and variables that provide minimal information are penalized and removed. In our customized LASSO step, a pre-defined top N (default setting is 10) is used to select a stable list of variables instead of LASSO's variable selection method. To do that, we record numbers of variables for all lambda simulations, keep only the repeated numbers, then select the repeated number that is closest to the pre-defined top N.

The 2nd step is single variable selection which aims to salvage any variables that were excluded by LASSO as they shared redundant information with the already selected variables. This is achieved using single independent variable regression, which can be performed using either a linear model, a generalized linear model, or a Cox model, depending on the type of outcome being studied. The 3rd step is stepwise model selection using the combined list of variables obtained from LASSO and single variable selection. The criterion for model comparison is the Akaike Information Criterion (AIC). The approach entails gradually building up a model by including or excluding one variable at a time. Ultimately, the model with the lowest AIC score among all potential models is chosen as the best model, and the variables in this model make up the final selected variable list.

### Example 8. Spatially Resolved Tumor Microenvironment Predicts Treatment Outcomes in Relapsed/Refractory Hodgkin Lymphoma

Summary: About a third of relapsed or refractory classic Hodgkin lymphoma (r/r CHL) patients succumb to their disease after high-dose chemotherapy followed by autologous stem cell transplantation (HDC/ASCT). Here, we aimed to describe spatially resolved tumor microenvironment (TME) ecosystems to establish new biomarkers associated with treatment failure in r/r CHL. Herein we performed imaging mass cytometry (IMC) on 169 paired primary diagnostic and relapse biopsies using a marker panel specific for CHL biology. For each cell type in the TME, we calculated a ‘spatial score’ measuring the distance of nearest neighbor cells to each “center” malignant Hodgkin Reed Sternberg cell within close interaction range. ‘Spatial scores’ were used as features in prognostic model development for post-ASCT outcomes. Highly multiplexed IMC data revealed shared TME patterns in paired diagnostic and early relapse/refractory CHL samples, whereas TME patterns were more divergent in pairs of diagnostic and late relapse samples. Integrated analysis of IMC and single cell RNA sequencing data identified unique architecture defined by CXCR5+ HRS cells and their strong spatial relationship with CXCL13+ macrophages in the TME. We developed a prognostic assay (‘RHL4S’) using four spatially resolved parameters, CXCR5+ HRS cells, PD1+CD4+ T cells, CD68+ tumor-associated macrophages, and CXCR5+ B cells, which effectively separated patients into high-risk vs low-risk groups with significantly different post-ASCT outcomes. The RHL4S assay was validated in an independent r/r CHL cohort using a multicolor immunofluorescence assay. Hence overall, we identified the interaction of CXCR5+ HRS cells with ligand-expressing CXCL13+ macrophages as a prominent crosstalk axis in relapsed CHL. Harnessing this TME biology, we developed a new prognostic model applicable to r/r CHL biopsies, RHL4S, opening new avenues for spatial biomarker development. Our study highlights the contrast between relatively persistent early relapse biology and more divergent late relapse biology with dynamic changes in the TME. Our data shed light on the unique and targetable spatial interaction between CXCR5+ HRS cells with CXCL13+ macrophages as a characteristic feature of relapsed/refractory CHL. To translate these biologic findings into the clinic, we developed and validated a spatially resolved biomarker assay (‘RHL4S’).

### Introduction

Microenvironment biology has been extensively explored in various cancers, including lymphomas, and many studies have revealed the pathologic importance of reactive immune cells in the tumor-microenvironment (TME). Classic Hodgkin Lymphoma (CHL) is characterized in that the malignant Hodgkin and Reed Sternberg (HRS) cells are greatly outnumbered by reactive, non-neoplastic cells in the TME. Typically, the malignant HRS cells represent less than 1% of cells in an individual tumour. Despite progress in elucidating TME biology and the extensive cellular crosstalk by cytokines/chemokines in CHL pathogenesis, the molecular determinants of treatment failure remain mostly unknown.

Despite recent treatment advances, about a third of relapsed and refractory (r/r) CHL patients succumb to their disease after high-dose chemotherapy followed by autologous stem cell transplantation (HDC/ASCT). Recent studies have reported that gene expression signatures representing non-neoplastic cells of the TME are associated with outcomes following therapy. Recent progress with multiplex imaging techniques has permitted comprehensive spatial characterization of TME biology, suitable for formalin-fixed paraffin-embedded tissue (FFPET). We therefore conceived that the more detailed description of spatially resolved ecosystems in samples at the timepoint of clinical relapse would lead to the development of refined biomarkers with the potential to improve risk stratification for post-secondary treatment outcomes.

Here, we performed IMC analysis of 71 paired pre-treatment/relapse biopsies and non-relapse biopsies (n=22) to describe the unique changes in microenvironment architecture linked to relapse on a per-patient basis. Our data shed light on the unique interactions between cancer cells and the TME, highlighting the spatial interaction between CXCR5+ HRS cells with CXCL13+ macrophages as a characteristic feature of r/r CHL. Consequently, we developed a new spatially resolved prognostic biomarker assay, “RHL4S”, that was also translated into a multi-color immunofluorescence (MC-IF) assay suitable for future implementation in routine pathological workflows.

### Patients and Techniques

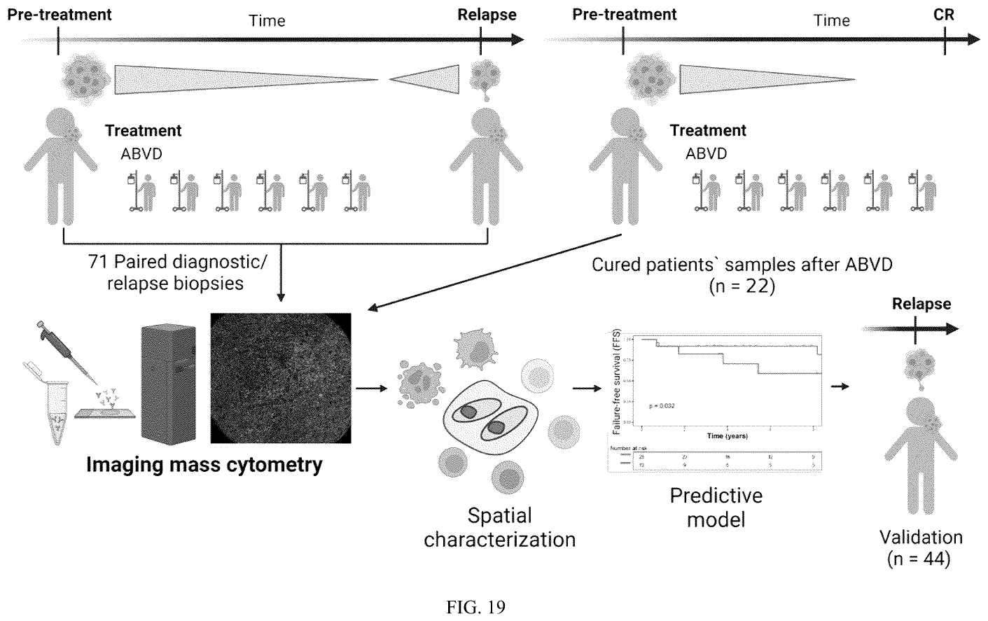

We analyzed IMC data using a customized marker panel for CHL from 165 CHL samples, including 71 patients with paired primary diagnostic and relapse specimens and 22 diagnostic control samples without relapse, termed the ‘discovery cohort’ (FIG. 19). Biopsies of these CHL cases, treated at BC Cancer between 1985 and 2011, are part of a tissue microarray (TMA). Patients were classified as having early relapse if their CHL progressed within 12 months after initial diagnosis or was refractory to first-line treatment.

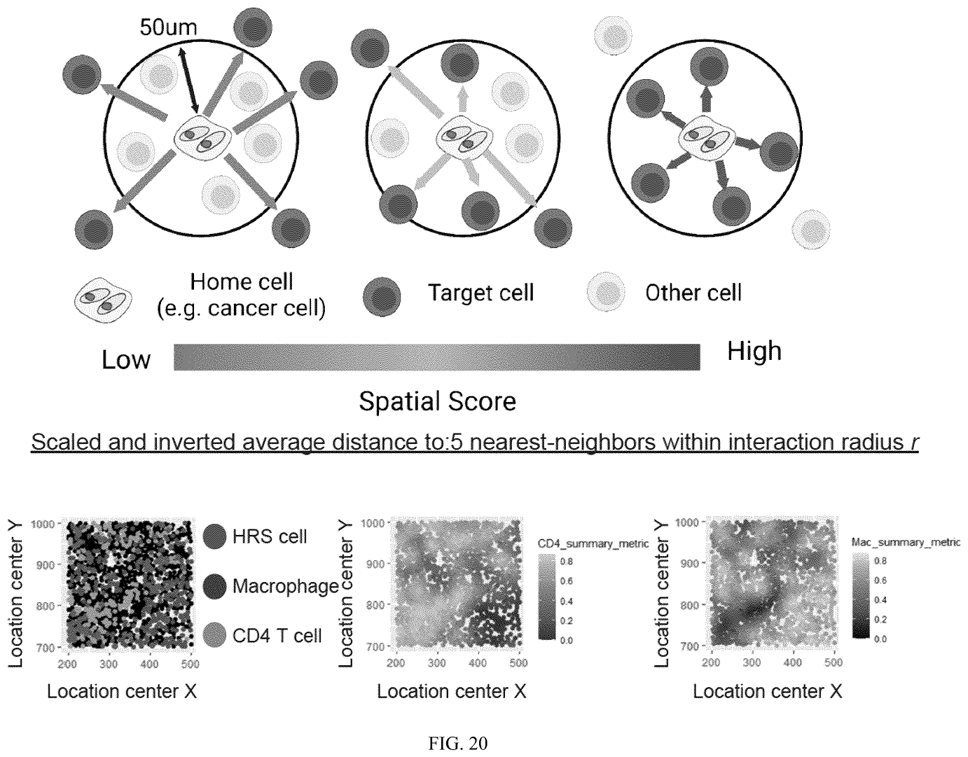

To develop a prognostic model for r/r CHL, we first calculated a ‘spatial score’ for each cell type using IMC and MC-IF data, defined as the term: (1-average distance of HRS cells to the 5 nearest neighbor cells of that type capped at 50 micrometers) to distinguish relationships between HRS cells and clusters of interacting cells. This strategy allowed us to quantify the spatially resolved cellular architecture in CHL (FIG. 20). Then, we applied our new cross-format LASSO (Least Absolute Shrinkage and Selection Operator) plus algorithm on the combination of two separate lists of standardized ‘spatial scores’ and standardized traditional protein-based cellular abundance percentages. Using the spatial scores of the four selected variables associated with post-ASCT failure free survival (FFS), we developed a risk classification prediction model, RHL4S based on our new XGpred algorithm in cvmpv R package (github.com/ajiangsfu/csmpv), which distinguished high and low-risk groups of patients with respect to post-ASCT FFS.

We then assembled a second, independent cohort for validation, termed the ‘validation cohort’ (n=44) used for MC-IF using a simplified panel which contains the multiparametric and spatial information of the 4 variables from the IMC-based model to establish a prognostic model that can be generalized and is applicable to routine pathology procedures. For the validation cohort, we selected r/r CHL patients treated at BC Cancer between 2012 to 2021 with available FFPET relapse biopsies according to the same selection criteria as the discovery cohort. To confirm the technical concordance between spatial scores derived from IMC vs MC-IF, we applied the MC-IF panel to a subset of the discovery cohort (N=19). To adjust RHL4S scores between IMC and MC-IF methodologies, calibration was performed between the two techniques correcting scores by a calibration value defined as the mean difference between RHL4S IMC and MC-IF scores.

Single cell RNA sequencing (scRNA-seq) were performed using sorted enriched HRS cells from cell suspensions of CHL. To boost transcriptome information for cell-to-cell interaction analyses, hybridization capture of marker genes was performed and run on Illumina Nextseq550. Genes used for hybrid capture sequencing of marker genes include: RC3H1, TIA1, TGFB1, ATF4, PTDSS1, BCL2, TNFRSF14, TNFSF10, STAT1, IL4R, BTLA, LGALS9, CD44, CCR7, CXCR4, CD69, BATF, MYC, RC3H2, TMEM30A, CXCR5, SPN, ID3, PLSCR1, CD38, FOS, GATA3, FOSB, SELPLG, CD28, CD5, PLSCR3, CD27, CCR6, SELL, IRF4, CXCR3, CD4, TCF3, FAS, ICOS, CD200, CD40LG, TNFRSF4, TNF, TCF4, CIITA, ADORA2A, IL7R, LGMN, IGHM, SPIB, TOX, HLA-DRB1, LAG3, PAX5, CD226, LGALS1, CD40, BCL7A, HLA-DRA, CTLA4, BLNK, CCR4, CD2, HLA-DRB5, CDKNIA, TNFRSF18, CD24, HMGB1, CD1C, CD79A, IGHD, LGALS3, TOX2, TIGIT, KLRG1, HLA-DQA1, PRDM1, PRF1, IL32, BCL6, CD7, CD22, CCL5, TLR1, IL2RA, ASNS, PDCD1, TBX21, EBI3, CD86, XKR8, CD8B, PTPRC, CXCL13, GZMK, CD19, CD3E, TNFSF14, IL21, TNFRSF13B, CD8A, CSF1, IKZF2, CCR5, MS4A1, NKG7, CXCR6, IGHG1, HAVCR2, FOXP3, CCL4, CD1D, IL411, GZMA, CD68, IL15RA, KLRD1, IGHA1, TLR6, LILRA4, IL6, TLR9, MKI67, IFNG, ITGAM, TLR7, GZMB, EOMES, FCERIG, TIMD4, PTDSS2, ITGAX, TNFRSF8, IL13RA1, FASLG, GNLY, CXCL10, IGHA2, SDC4, IL10, IL3RA, ZBTB16, TNFRSF9, CD274, CEACAM1, KLRC1, CD160, RORC, TNFAIP2, S100A9, NECTIN2, CLEC4C, CSFIR, CD80, PDCDILG2, FCGR3A, SH2D1B, IL1B, NCR1, FUT4, CD33, CD1E, NCAM1, KLRC2, CD14, SDC1, CXCL9, CCL17, CD163, PRAME, IL13, IL4, PIGR, CD34, and CXCL12.

Written informed consent or consent waivers were obtained for all patients. This study was reviewed and approved by the University of British Columbia-BC Cancer Agency Research Ethics Board (H14-02304), in accordance with the Declaration of Helsinki.

### Results

### The Spatially Resolved TME of r/r CHL

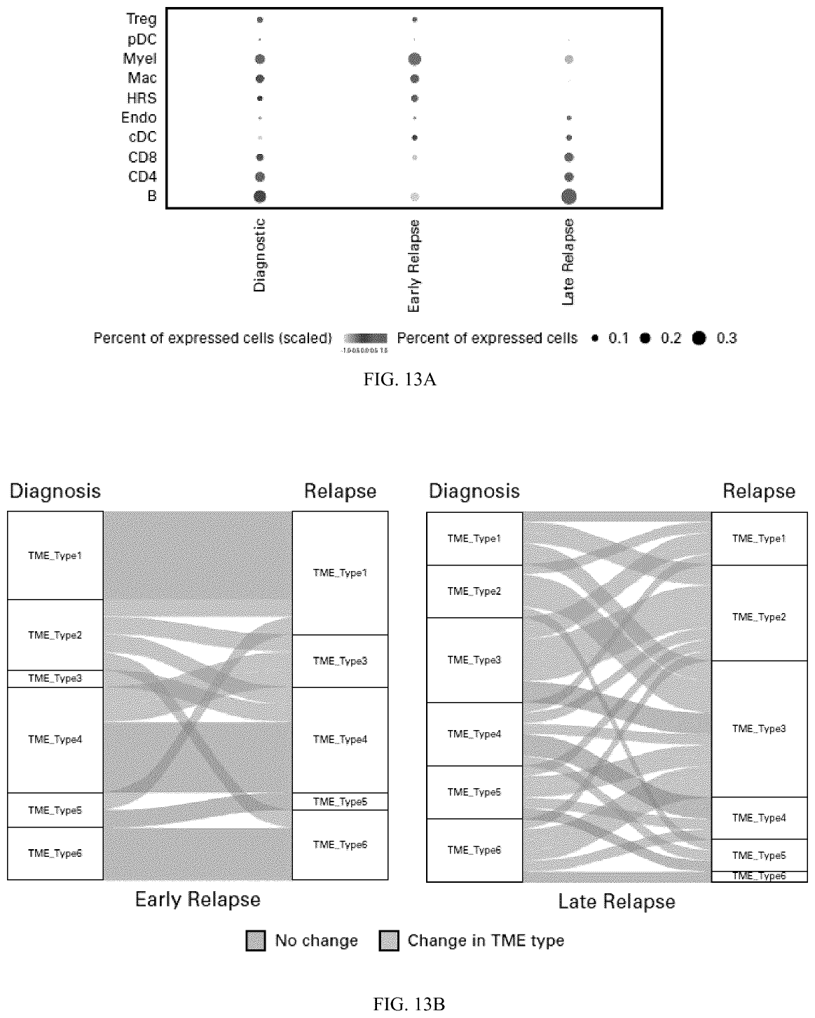

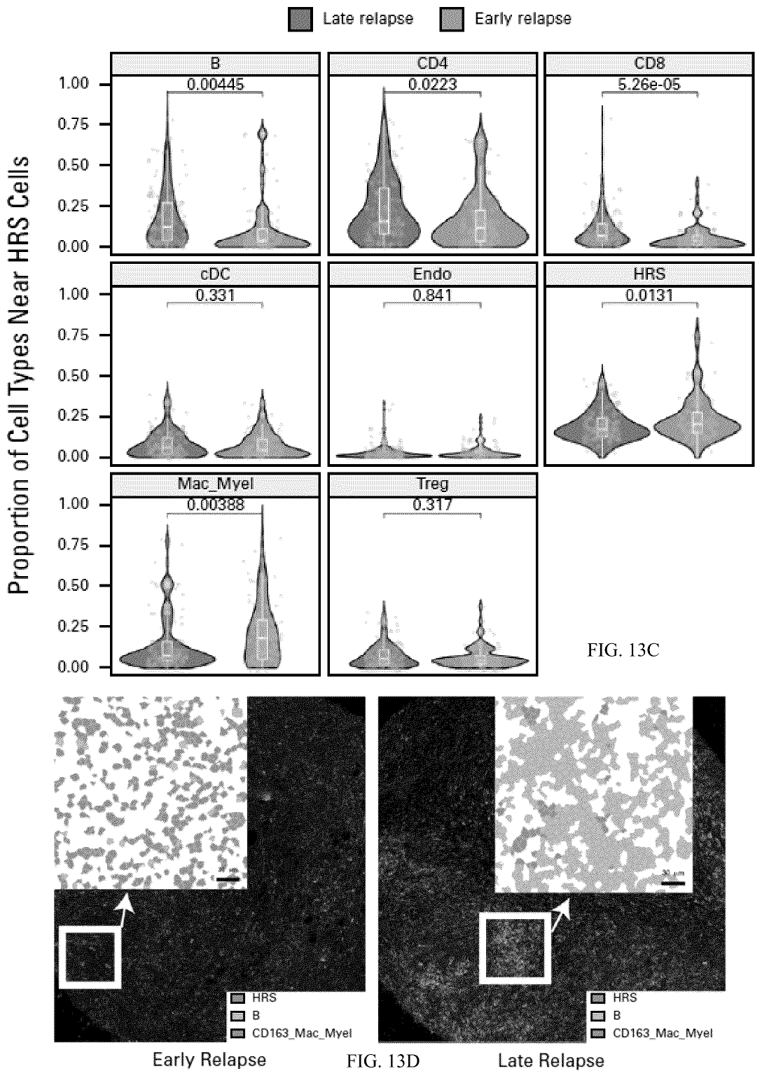

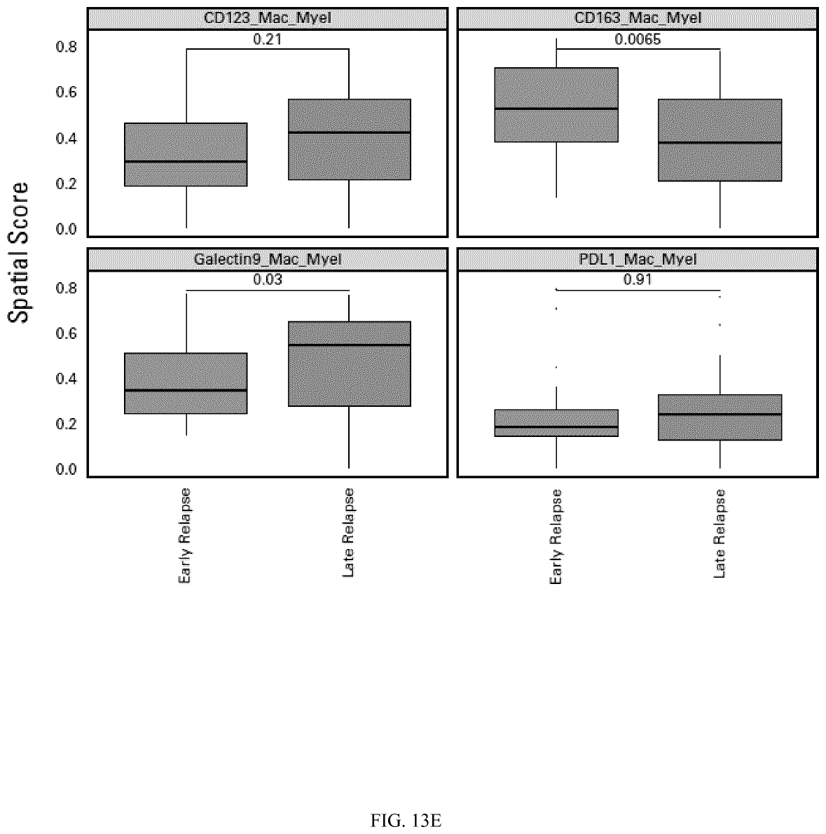

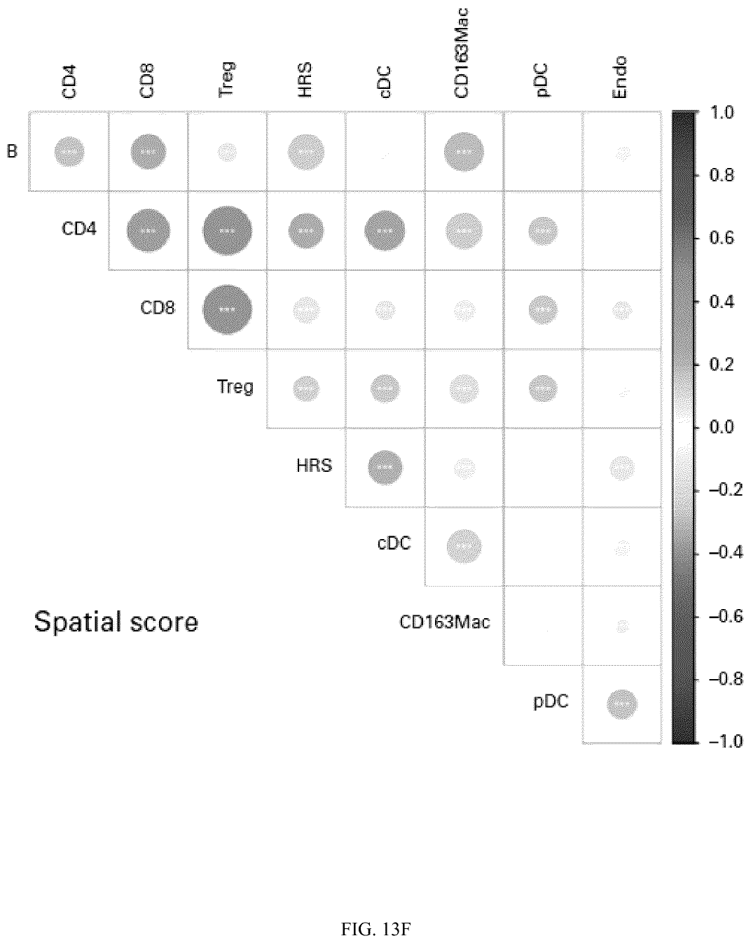

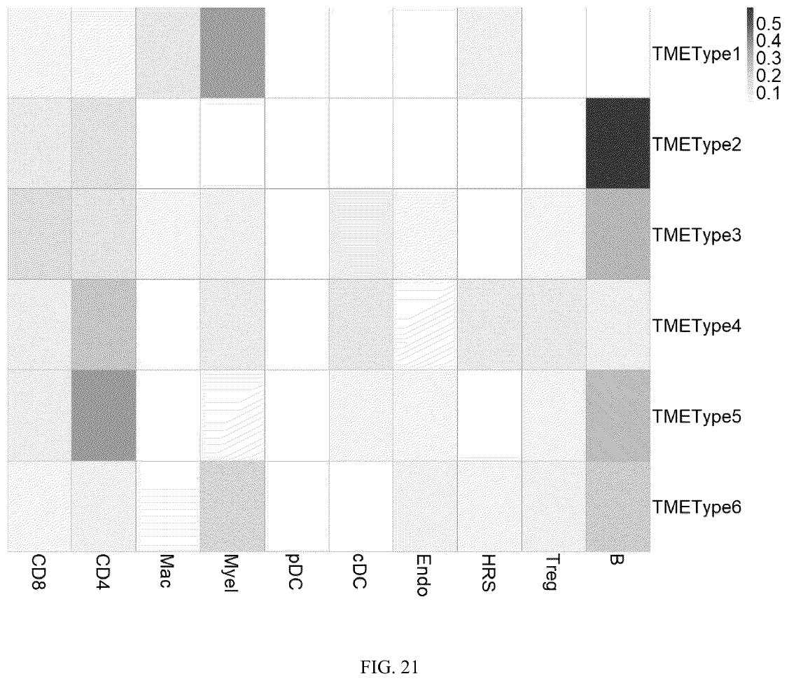

We obtained highly multiplexed images for a total of 7,146,042 cells at 1 μm² resolution. To define the spatial architecture and related cellular interactions, we first investigated the differences in TME characteristics according to disease status (initial diagnosis vs early relapse vs late relapse), showing differential abundance of each immune cell type (FIG. 13A). While diagnostic samples demonstrated known CD4 T cell enrichment patterns, relapse samples showed a unique immune cell abundance profile. Since high-resolution characterization beyond cellular abundance is needed to delineate the complexity of the cellular ecosystem in the TME, we defined comprehensive TME ecotypes using unsupervised clustering by leveraging a multitude of TME features (FIG. 21). When determining TME ecotypes in individual diagnostic and relapsed biopsy pairs per patient, we observed significant differences in TME dynamics between early relapse and late relapse samples, with a significantly increased number of TME type transitions in late relapse (p<0.01) (FIG. 13B). Late relapse samples were characterized by an abundance of non-malignant B cells (P<0.01) along with CD4 (P<0.05) and CD8 T cell enrichment (P<0.01) (FIGS. 13A, 13C and 13D). Conversely, CHL biopsies from patients with early relapse demonstrated more similar TME patterns between diagnostic and relapse samples (FIG. 13B). In particular, the macrophage/myeloid cell-enriched TME ecotypes 1 and 6 were constant over time between diagnostic and early relapse biopsies (FIGS. 13B and 13C). Further analyses revealed that a CD163+ macrophage population, indicative of M2 polarization, was significantly enriched in early relapse samples (FIGS. 13D and 13E). Consistent with these findings, spatial analyses of relapse samples revealed an inverse correlation of CD163+ macrophages and B cells in cellular neighborhoods (FIG. 13F).

The Unique Spatial Architecture Associated with CXCR5+ HRS Cells in Relapse Biopsies.

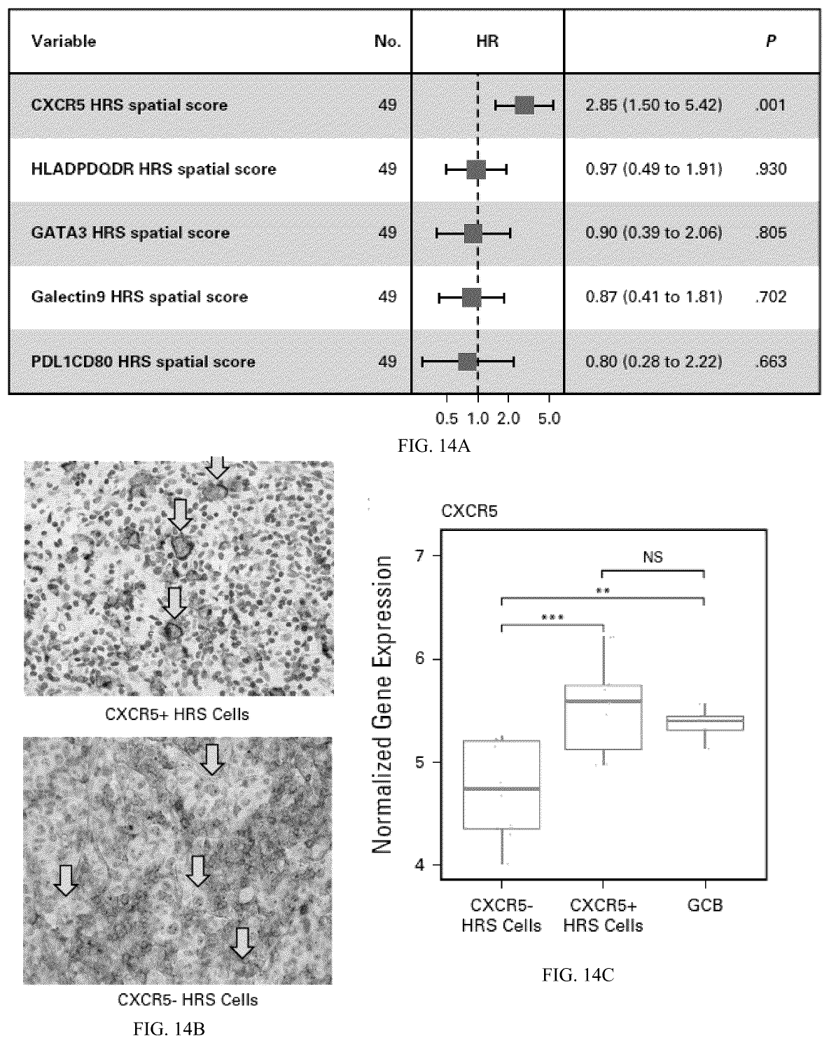

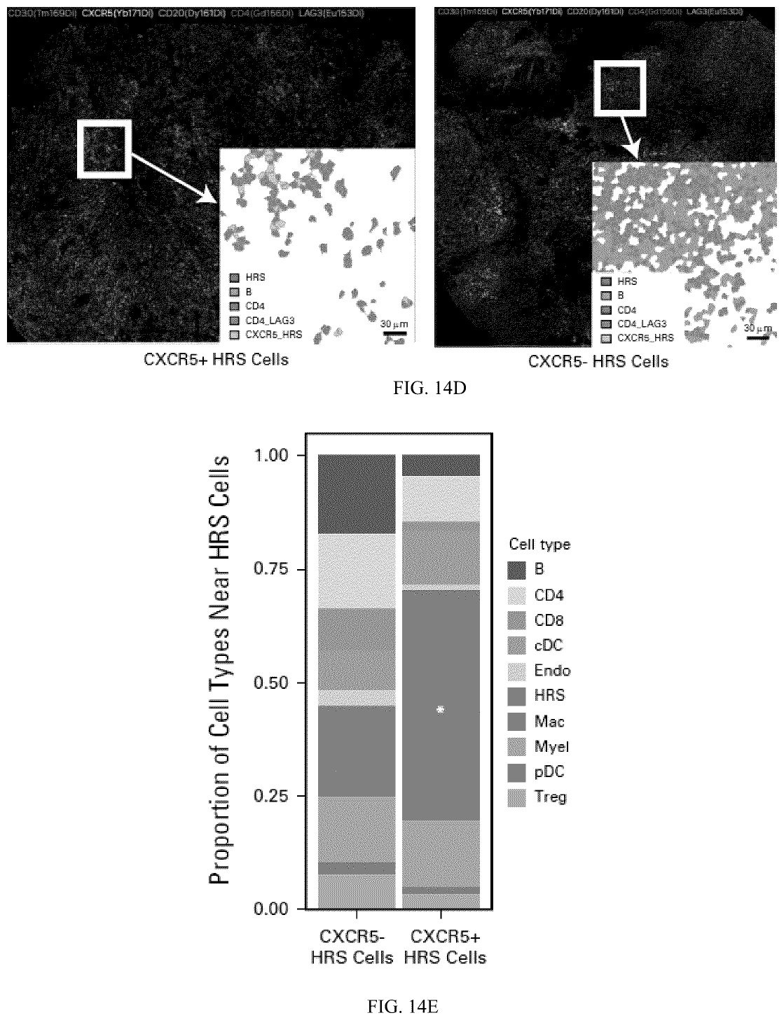

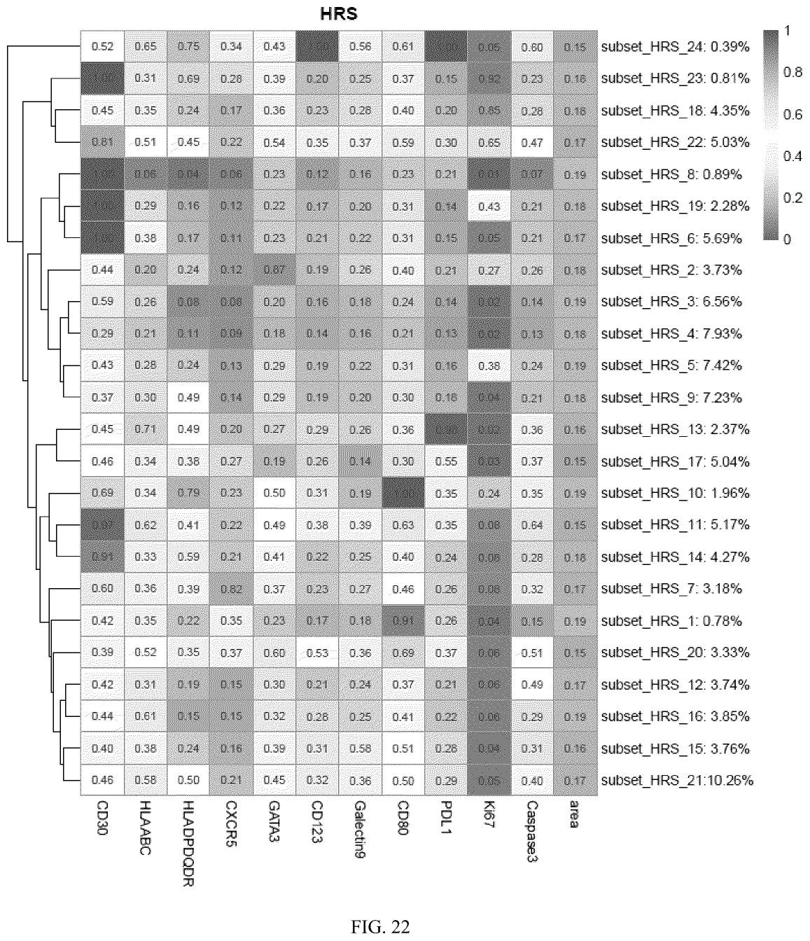

In previous studies, the most prominent obstacle for biomarker development in CHL was the scarcity of the malignant HRS cells and the heterogeneity of TME composition within individual tumor biopsies. Our IMC panel was designed to simultaneously quantify protein expression on HRS cells and the TME, including known variably expressed markers on HRS cells, such as CD30, PD-L1 and major histocompatibility class I and II (MHC-I and MHC-II). Unsupervised clustering identified several new subsets within the HRS cellular compartment, defined by, for example, high GATA3 and CXCR5 expression. These phenotypic HRS cell definitions are additive to other subsets, such as HRS cells with high PD-L1 or CD123 expression (FIG. 22). Next, we evaluated the prognostic impact of these HRS features in the spatial context of r/r CHL. LASSO analysis identified CXCR5+ HRS cells as the most significant HRS cell phenotype correlated with post-ASCT FFS, in contrast to the absence of an outcome correlate with all HRS cells, not further specified (FIG. 14A). To confirm CXCR5 expression patterns on HRS cells by both protein and RNA levels, we assessed expression of CXCR5 using immunohistochemistry (IHC) and reanalyzed published Affymetrix gene expression data generated from micro-dissected HRS cells of primary HL samples. These analyses confirmed variable CXCR5 surface protein expression was well correlated with mRNA expression, and CXCR5 was highly expressed in a subset of CHL tumors (FIG. 14B) comparable to expression levels found in CD77+ germinal center B cells (FIG. 14C). Interestingly, CXCR5+ HRS cells were spatially arranged together with other CXCR5+ HRS cells forming cell clusters as an architectural feature (P<0.05). CXCR5+ HRS cell clusters were also characterized by a lower abundance of non-malignant immune cells, including CD4+ T reg cells and CD20+ B cells when compared to CXCR5-HRS cells (FIG. 14D-14F), indicating distinct TME characteristics associated with CXCR5+ HRS cells.

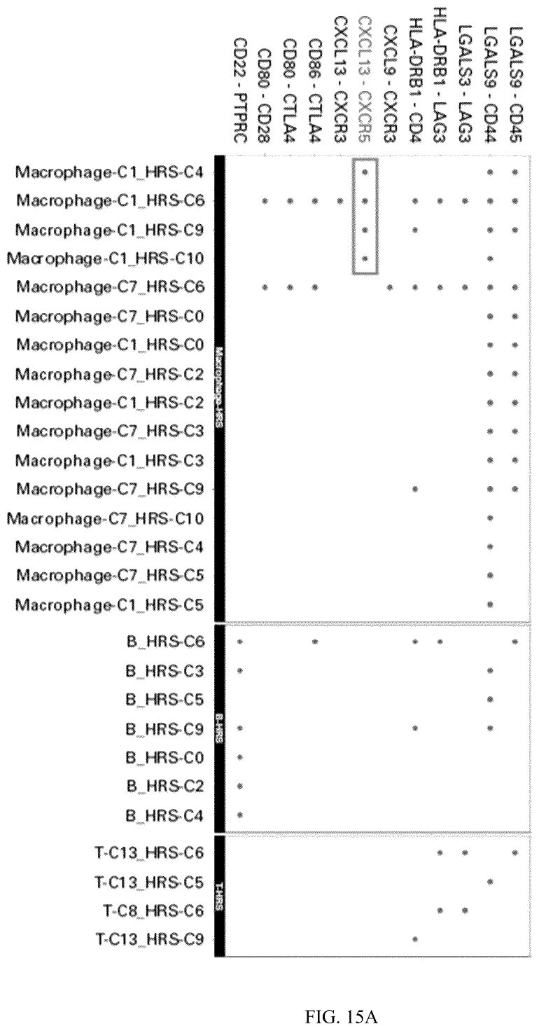

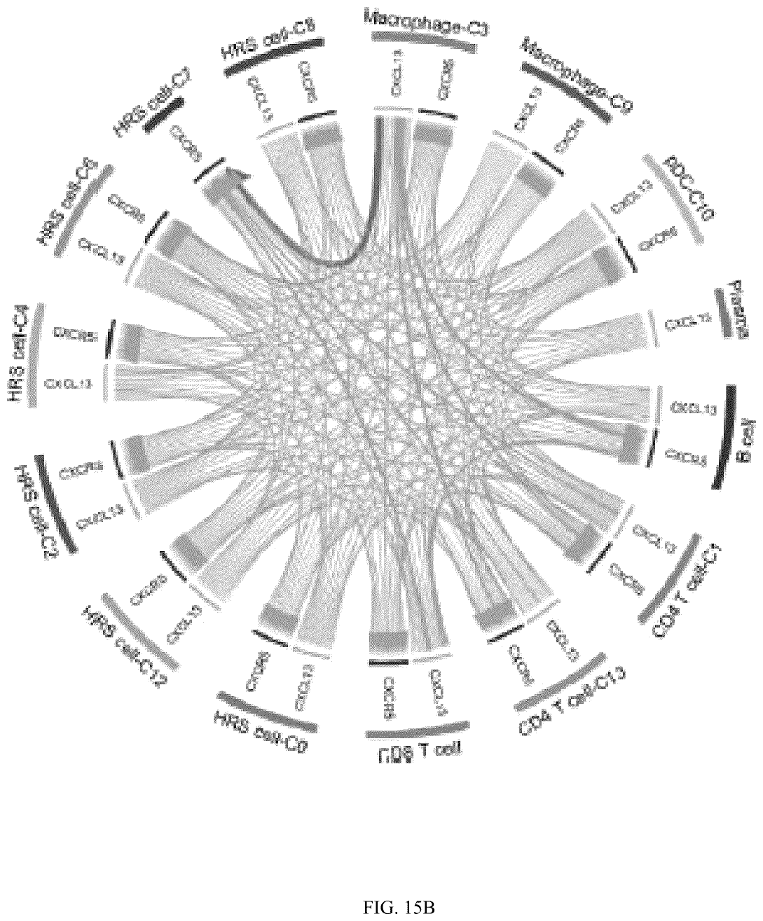

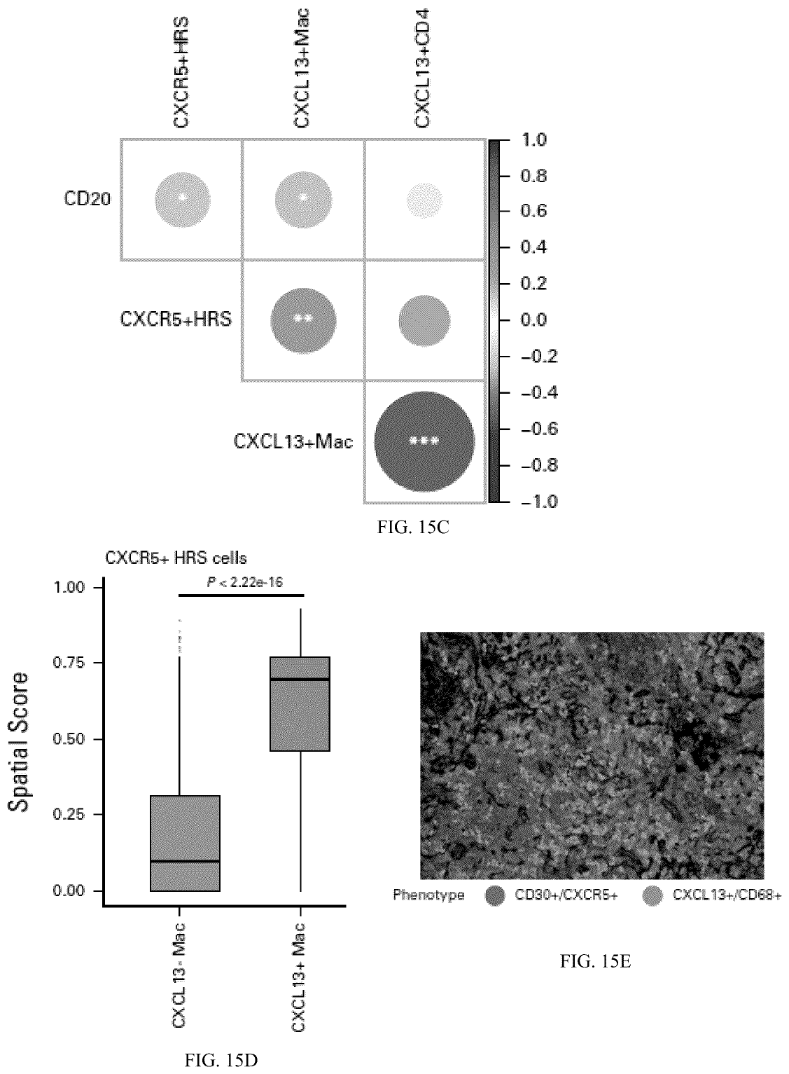

We next sought to understand the cellular interactions between CXCR5 HRS cells and other immune cell populations. Although we observed fewer TME components surrounding CXCR5 HRS cells in the IMC data (FIG. 14E), we conceived that previously unknown immune cell populations, which cannot be defined with the current IMC panel, might interact with CXCR5 HRS cells. Therefore, we additionally performed scRNA-seq on CHL samples with partial enrichment of the HRS cell population by cell sorting. Using the cell-to-cell communication tool, Cell Chat (described by Jin S et al. in Nat Commun 12:1088, 2021), we predicted CXCR5-CXCL13 interaction between CXCR5 HRS cells and CXCL13 macrophages (FIG. 15A). This significant interaction was also validated by the alternative iTALK method (described by Wang Y et al. in BioRxiv, 2019) (FIG. 15B). CXCL13 is a cell attractant via the CXCL13/CXCR5 axis. Intriguingly, CXCL13+ macrophages mostly (>99%) did not co-express M2 macrophage markers, such as CD163 or CD206, indicating a distinct profile of this population. To further describe the spatial relationship between CXCR5+ HRS cells and CXCL13+ macrophages, we applied multicolor immunofluorescence (MC-IF) on the discovery cohort TMA that was used for IMC. Consistent with the results of the scRNAseq-based cell-to-cell interaction prediction, we observed a strong positive correlation of the abundance of CXCR5+ HRS cells with CXCL13+ macrophages in the MC-IF data (FIG. 15C). Furthermore, CXCL13+ macrophages were located in close proximity to CXCR5+ HRS cells (FIGS. 15D and 15E), supporting the importance of the CXCR5/CXCL13 axis in CHL.

### Development of a Prognostic Model Leveraging Spatial TME Information

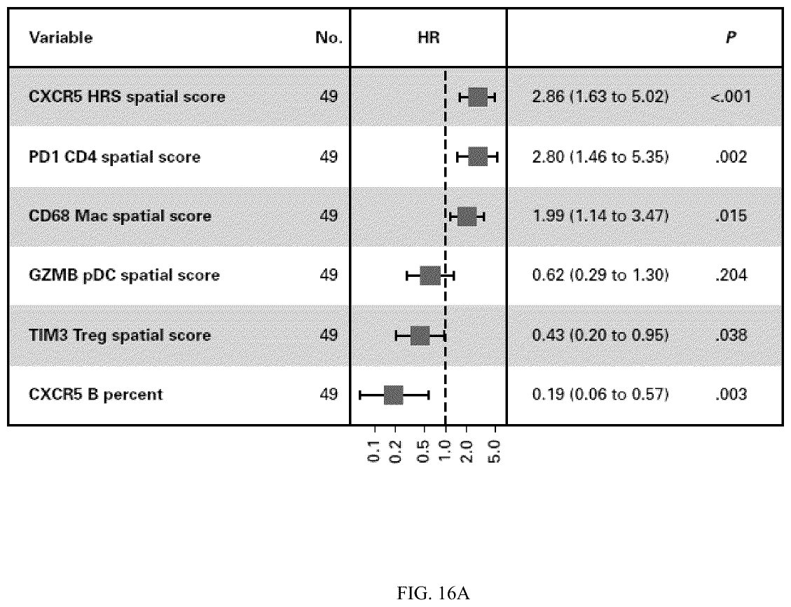

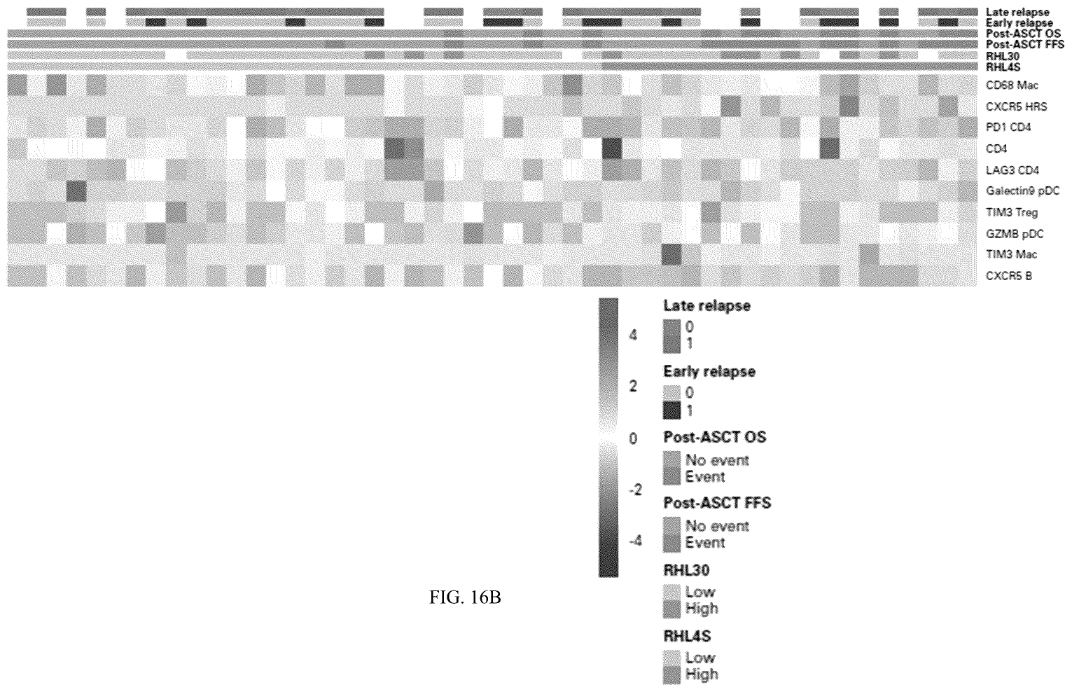

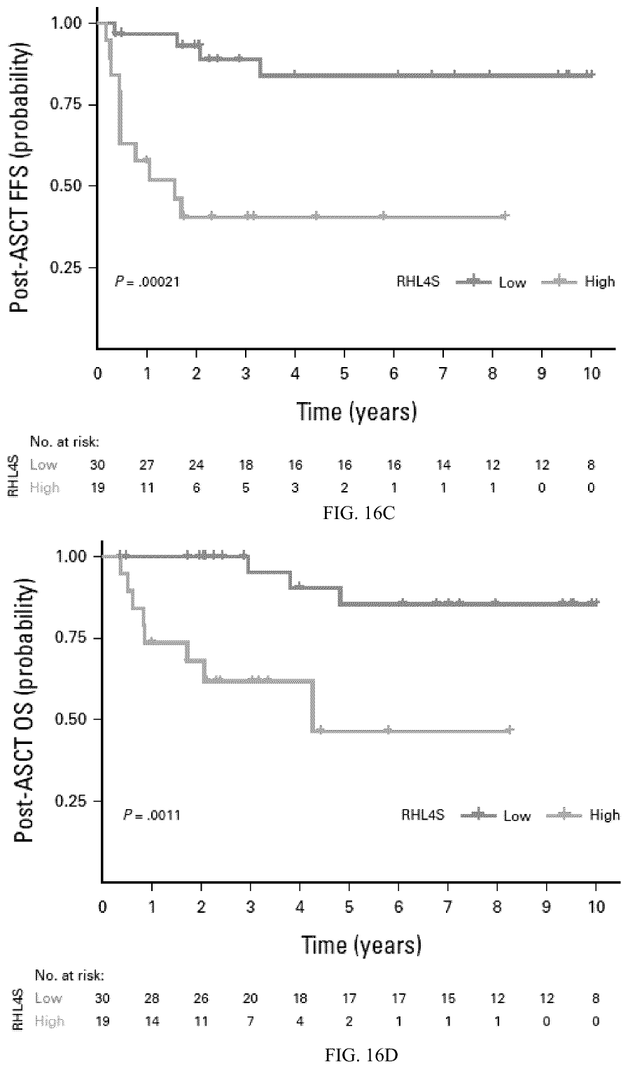

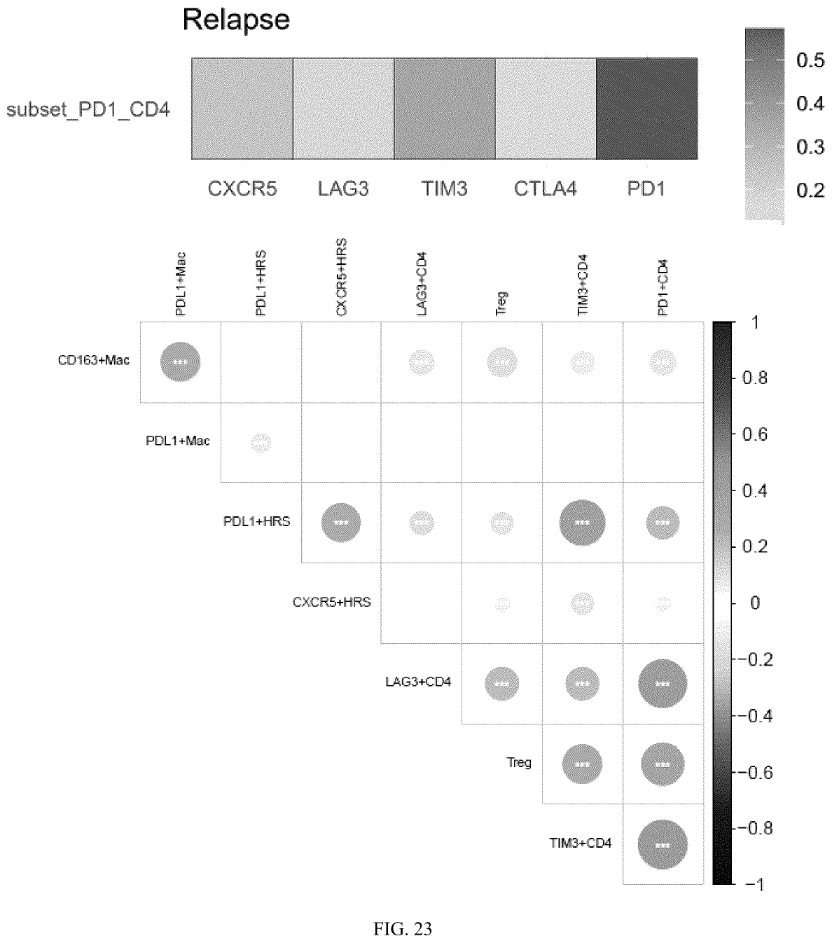

Next, we sought to construct a prognostic model for post-ASCT outcomes in r/r CHL patients taking advantage of simultaneously capturing HRS cell and TME biology by IMC. We focused our analyses on biomarker measurements in relapse samples. For feature selection, we first performed cross-format LASSO plus analysis on two separate sets of variables, namely standardized spatial scores and conventional cellular abundance percentages (based on protein expression markers). Strikingly, all top five variables, which were significantly associated with post-ASCT FFS p-values <=0.05, were ‘spatial scores’ (FIG. 16A), confirming the importance of spatially-informed parameters for outcome prediction. Consistent with the independent prognostic importance of these variables (FIG. 16A), each patient sample showed distinct spatial patterns which were linked to these cellular components (FIG. 16B). Four variables, CXCR5+ HRS cells (hazard ratio [HR] (95% CI): 2.86 (1.63-5.02)), PD1+CD4+ T cells (HR (95% CI): 2.80 (1.46-5.35)), CD68+ macrophages (HR (95% CI): 1.99 (1.14-3.47)) and CXCR5+ B cells (HR (95% CI): 0.19 (0.06-0.57)) were identified as factors most significantly associated with post-ASCT FFS. In contrast, we could not observe any significant prognostic associations of the TFH subset or other macrophage subsets such as CD163+ macrophages and PD-L1+ macrophages. The CD4+PD1+ T cell population co-expressed other inhibitory receptors such as TIM3 and showed a significant positive correlation with PD-L1+ HRS cells (FIG. 23).

To leverage the multifactorial spatial biologic features which are linked to relapsed/refractory biology, we developed a risk prediction model, RHL4S, using the spatial scores from these four variables. RHL4S identified a high and low-risk group of patients with the high-risk group of patients having significantly inferior post-ASCT FFS (5-year post-ASCT FFS: 41% vs 81%; P<. 0001; FIG. 16C) and inferior post-ASCT overall survival (OS) (5-year post-ASCT OS: high-risk, 46% vs low-risk, 85%; P=001; FIG. 16D).

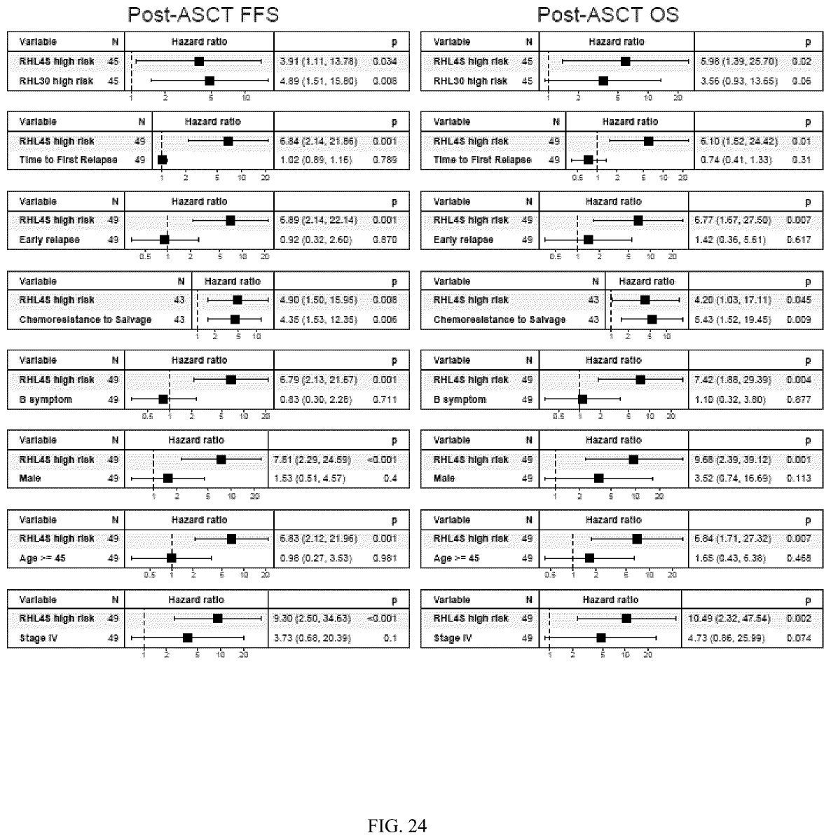

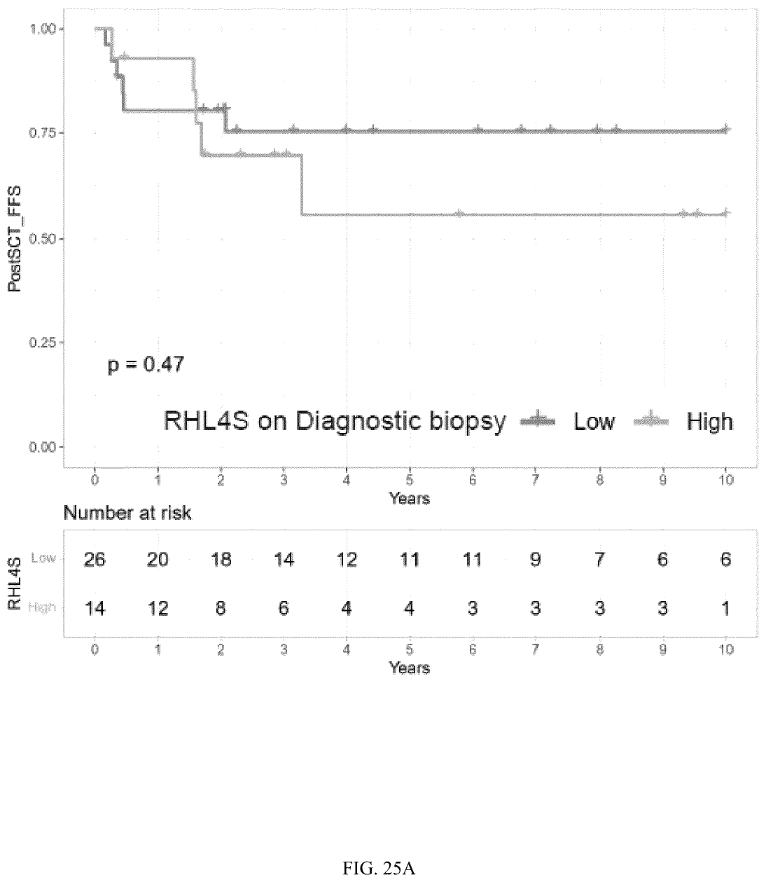

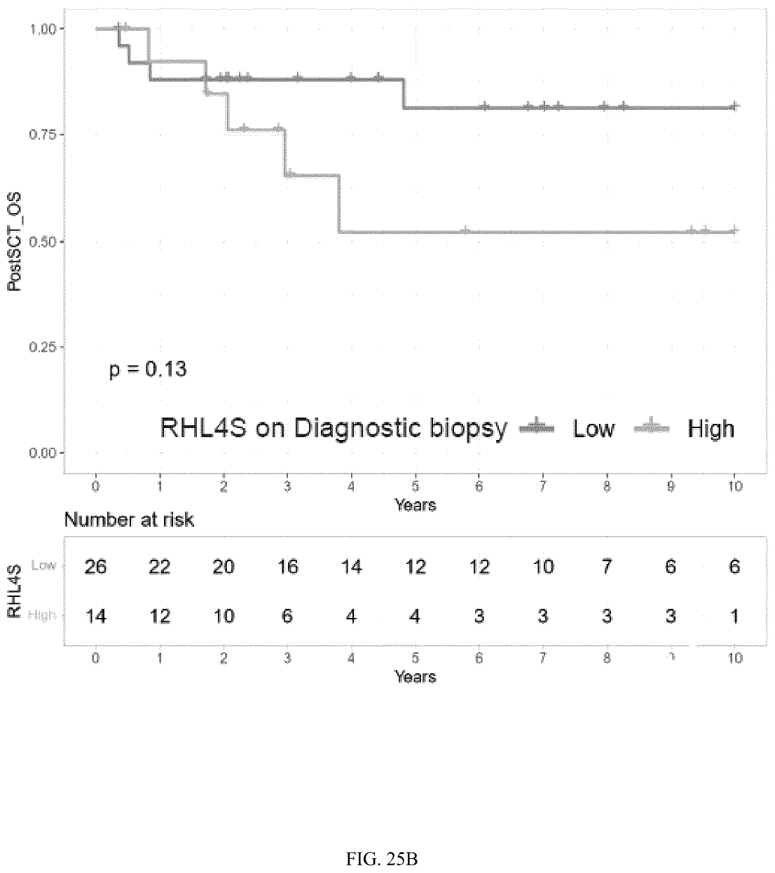

We then investigated the independent prognostic value of RHL4S with respect to previously reported prognostic factors of post-ASCT outcomes including RHL30 (FIG. 16B), time to first relapse, chemo-resistance, B symptoms at relapse, age ≥45 years at ASCT, and stage IV disease at initial diagnosis. Pairwise multivariable Cox regression analysis including RHL4S and these known prognostic variables demonstrated that the RHL4S risk group was statistically independent (P<. 05) (FIG. 24). RHL4S scores using expression measurements from the initial diagnostic biopsy could not predict post-ASCT outcome, confirming the superiority of relapse biopsies for predicting post-ASCT outcomes in r/r CHL (FIG. 25).

### Independent Validation for RHL4S

To establish a prognostic model that can be readily used for diagnostic procedures in clinical practice, we next translated the IMC-based RHL4S assay into MC-IF methodology. We built a simplified marker panel encompassing the four expression variables of the predictive model and calculated calibrated individual spatial scores from the MC-IF data in the independent validation cohort of relapse biopsies from 44 r/r CHL patients uniformly treated with salvage treatment and consolidating ASCT.

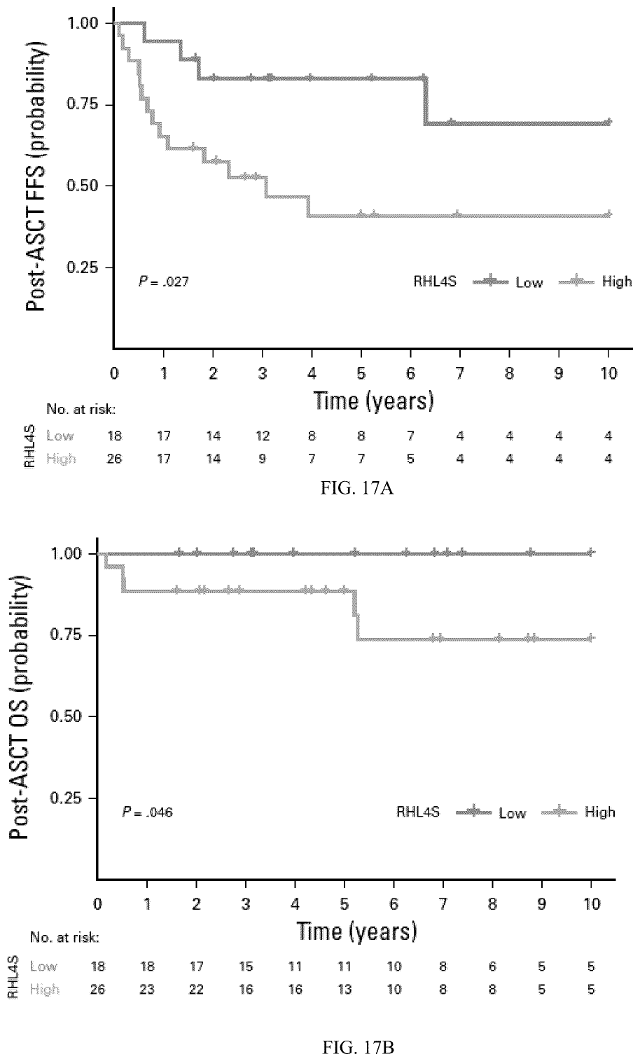

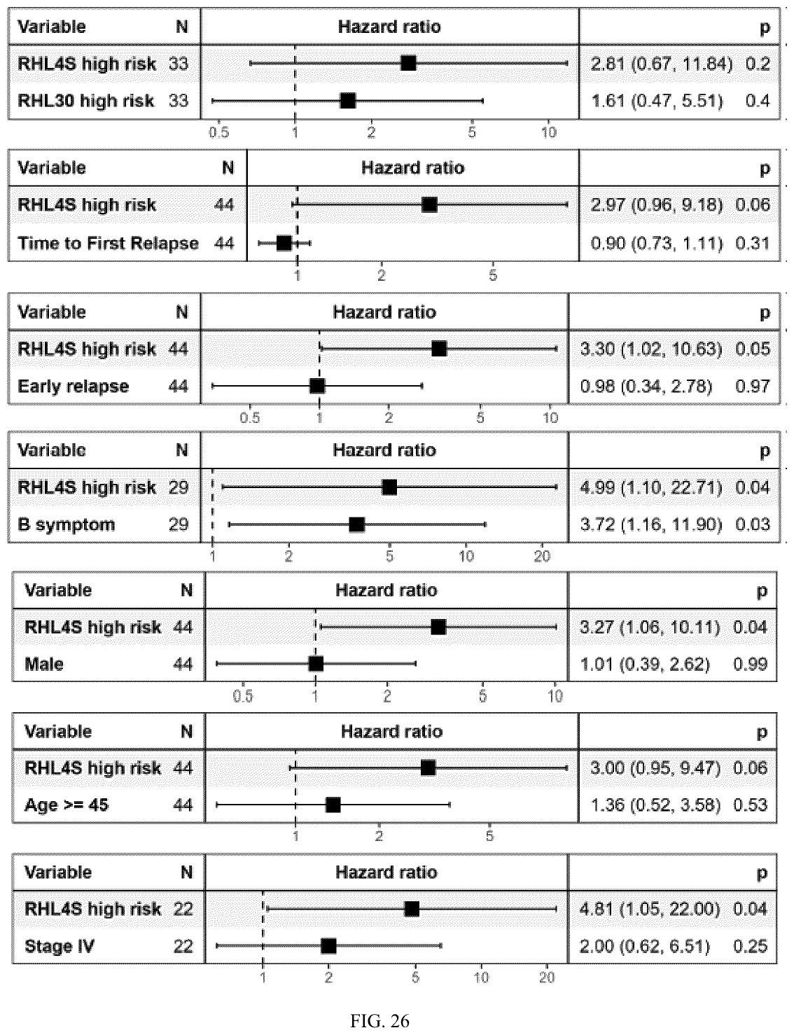

Salvage treatment for relapsed or refractory Hodgkin lymphoma included: a combination of gemcitabine, dexamethasone, and cisplatin (GDP); a combination of ifosfamide, carboplatin, and etoposide phosphate (ICE); or a combination of cyclophosphamide, oncovin, procarbazine, and prednisone (COPP). In the validation cohort, first-line brentuximab vedotin (BV) plus AVD was used for only one patient and ten patients received BV consolidation while no patients in this study received PD-1 blockade before ASCT. Consistent with the finding in the discovery cohort, high-risk patients displayed unfavorable post-ASCT FFS (5-year post-ASCT FFS: 41% vs 83%; P=. 027; FIG. 17A) and post-ASCT OS (5-year post-ASCT OS: 88% vs 100%; P=. 046; FIG. 17B) in the independent validation cohort. Finally, we investigated the independent prognostic value of RHL4S with respect to previously reported prognostic factors of post-ASCT outcomes in the validation cohort. Pairwise multivariable Cox regression analysis demonstrated the independence of RHL4S (P<. 05) or trends toward independence against these markers (FIG. 26). Of note, although the mRNA gene-expression-based RHL30 assay showed similar performance to RHL4S in the discovery cohort, RHL30 did not show a significant difference in post-ASCT FFS between the high and low-risk groups in the validation cohort, indicating superior performance of RHL4S over RHL30.

### Discussion

Here, we developed and validated a new prognostic model, RHL4S, based on spatial TME biology, that can predict outcome after ASCT in patients with r/r CHL. Our study establishes a paradigm for the strong prognostic value of spatially resolved biomarkers over traditional expression-based biomarkers in lymphoma and indicates the biological importance of tumor architectural patterns for biomarker development and discovery of immunotherapeutic targets in other cancer types. Importantly, RHL4S was associated with post-ASCT outcomes regardless of relapse status (early relapse or late relapse).

We conceive that RHL-4S (github.com/ajiangsfu/RHL4S) is suitable for use not only on the MC-IF platform but also on other clinical practice platforms such as IHC or H&E.

RHL4S includes four spatially resolved variables, including macrophages that were identified as a prognostic biomarker in CHL based on raw cellular abundance in the TME in multiple studies. We now also identified a phenotypically defined subset of HRS cells (CD30+CXCR5+) that was associated with outcome, and intriguingly, we observed enrichment of CXCL13+ macrophages in regions surrounding CXCR5+ HRS cells. We also recently found that CXCL13+PD1+ TFH-like cells are enriched in a specific subtype of CHL (lymphocyte-rich CHL) and associated with poor clinical outcome in this rare subtype. These two studies indicate the importance of ligand (CXCL13) and receptor (CXCR5) interaction and their association with treatment failure in CHL.

Our study also highlights the contrast between early relapse and late relapse biology. We observed relatively similar TME features between diagnostic and relapse biopsies in early relapse cases, while differences were more pronounced in late relapse cases. This finding raises the hypothesis that the establishment of malignant cellular ecosystems, including HRS cell-driven shaping of the TME, is persistent over time after first-line treatment in early relapse CHL, and by contrast, the biology of late relapses is indicative of more divergent disease and more dynamic changes in the TME.

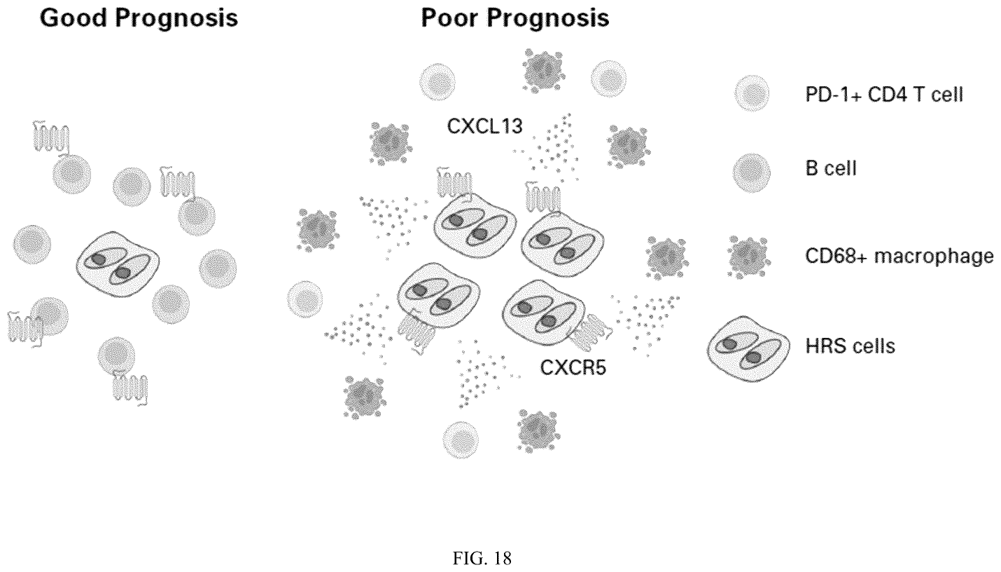

The multiple cellular components associated with treatment outcome strongly suggest that the mechanism underlying relapsed disease might not be uniform (FIG. 18). Nevertheless, delineating specific and targetable biology underlying relapsed/refractory disease, such as the PD1-PDL1 and CXCR5-CXCL13 axes, might lead to more effective delivery of precision oncology. Additionally, co-expression patterns of multiple co-inhibitory receptors including TIM3 and LAG3 in PD1+CD4+ T cell subsets might provide a rationale to consider multiple targeted treatments as investigated in clinical trials (e.g. ClinicalTrials.gov identifiers: NCT05216835 and NCT03598608). The development of biology-driven biomarkers in CHL might also help pave the way for rational treatment selection, an approach that is currently emerging in diffuse large B-cell lymphoma based on gene expression and mutational profiling.

Our immunofluorescence-based RHL4S assay can be readily used for FFPE tissues collected according to trial correlative protocols and inform on important TME components in relapse biopsies of patients treated with BV and/or checkpoint inhibitors. Moving forward, we conceive that the value of spatially resolved biomarkers will be tested in additional lymphoma subtypes and other cancers with a biologically important TME component.

Single-cell RNA-seq counts (generated using Cell Ranger v2.1.0) and a merged SingleCellExperiment R object are available in the European Genome-phenome Archive (EGA; EGAD00001010892) via controlled access. IMC data are available at zenodo.org/deposit/7963681. R packages, csmpv (github.com/ajiangsfu/csmpv), and RHL4S (github.com/ajiangsfu/RHL4S) are available on GitHub.

In various embodiments, RHL4S is a prognostic model developed for patients with relapsed or refractory classic Hodgkin lymphoma (r/r CHL) who have undergone high dose chemotherapy followed by autologous stem cell transplantation (HDT/ASCT). The model is based on imaging mass cytometry (IMC) and incorporates information about cellular interactions, specific expression features of Hodgkin and Reed-Sternberg (HRS) cells, and the spatial architecture of the tumor microenvironment (TME). The study found that CXCR5+ HRS cells, PD1+CD4+ T cells, macrophages, and CXCR5+ B cells were significantly associated with post-ASCT failure-free survival. The RHL4S model was found to be more effective than classical protein percentage-based models in predicting outcomes in r/r CHL patients.

The RHL4S prognostic model is based on four spatial score variables that are inflated into a 0-100 scale range: CXCR5 HRS spatial score, PD1 CD4 spatial score, Mac spatial score, and CXCR5 B spatial score. The model was built using our new XGpred algorithm, which combines the machine learning method XGBoost with traditional statistical techniques such as model-based clustering, spline regression, LPS (Linear Prediction Score), and empirical Bayesian approaches. XGPred functions will be added into R package csmpv at github.com/ajiangsfu/csmpv.

To make the model applicable to patients in daily clinical practice, the findings from IMC were translated into simplified data. A simplified panel was built to obtain information on the four variables from the predictive model and calculate the spatial score from MC-IF data. Calibration was performed by applying this panel to a subset of the IMC cohort, confirming the concordance between spatial score from IMC and MC-IF, and setting the optimal cut-off on MC-IF for RHL4S assay. The mean difference in RHL4S scores between IMC and MC-IF for the calibration cohort was used as a parameter to adjust RHL4S scores for any MC-IF validation cohorts.

The current RHL4S R package calculates RHL4S scores and predicts RHL4S risk group classification based on MC-IF data.

### Additional Details:

### Tissue Samples

We analyzed IMC data from 164 CHL samples, including 71 patients with paired primary relapse specimens and 22 diagnostic control samples without any relapse, termed the ‘discovery cohort’ (FIG. 19). Biopsies of these CHL cases, treated at BC Cancer between 1985 and 2011, are part of a tissue microarray (TMA) that was previously reported by Chan et al. in J Clin Oncol 35:3722-3733, 2017. In brief, the patients were selected according to the following criteria: patients received first-line treatment with doxorubicin, bleomycin, vinblastine, and dacarbazine (ABVD) or ABVD-equivalent therapy with curative intent; patients experienced CHL progression despite primary treatment (i.e., occurrence of refractory disease or cHL relapse); and tissue derived from paired diagnostic and relapsed excisional biopsies was available.

Herein, a relapse specimen (or relapse biopsy) refers to a second biopsy taken at the time of emergence of either primary refractory lymphoma or relapsed cHL. In contrast, a diagnosis specimen (or diagnosis biopsy) refers to a first biopsy taken at time of diagnosis of HL.

Patients were classified as having primary refractory disease if their cHL progressed during ABVD treatment or within 3 months of finishing chemotherapy. Patients who had recurrence beyond 3 months of ending ABVD treatment were classified as having relapsed disease. Patients were classified as having early relapse disease if their CHL progressed within 12 months after initial diagnosis or refractory to first-line treatment.

Clinical evaluation and/or diagnostic imaging (mainly computed tomography) were used to assess response to salvage therapy. Patients with complete or partial response were classified as chemotherapy sensitive. Patients with stable or progressive disease were classified as chemotherapy resistant. All patients went on to transplantation irrespective of their response to salvage chemotherapy and hence only received one salvage regimen.

We also assembled a second, independent cohort for validation, termed the ‘validation cohort’ (n=44) used for multi-color immunofluorescence (MC-IF). For the validation cohort, we selected r/r CHL patients treated at BC Cancer between 2012 to 2021 with available FFPET relapse biopsies, according to the same selection criteria as for the discovery cohort. In addition, independent FFPET relapse biopsies from r/r CHL as part of the previously reported TMA in Chan et al. in J Clin Oncol 35:3722-3733, 2017 were including in the validation cohort. One patient in the validation cohort received first line brentuximab vedotin plus AVD. One and ten patients in the validation cohort, respectively, received first line brentuximab vedotin (BV) plus AVD and BV consolidation. No patients in this study received PD-1 blockading therapy prior to ASCT. For single cell RNA sequencing, three patients with histologically confirmed diagnostic CHL were included in this study. Patients were selected based on the availability of tissue that had been mechanically dissociated and cryopreserved as cell suspensions following diagnostic lymph node biopsy at BC Cancer.

### Tissue Microarray (TMA) Construction, Single Color IHC and EBER-1 ISH on TMA

For TMA construction in the validation cohort, 1.5 mm duplicate cores were obtained from representative areas containing Hodgkin Reed-Sternberg cells of 28 relapse biopsies of CHL. The diagnosis was made according to the WHO classification and reviewed by hematopathologists. For immunohistochemistry (IHC) staining, 4 μm slides of the TMA and antibodies were used. Staining was performed on a Benchmark XT platform (Roche Diagnostics, USA) or intelliPATH platform (Biocare Medical, USA). The slides were independently reviewed and scored and the single stain IHC scores were utilized for MC-IHC segmentation. Epstein-Barr virus-encoded small RNA 1 (EBER-1) in situ hybridization (ISH) was performed according to the manufacturer's protocol (Roche Diagnostics, USA).

### Imaging Mass Cytometry

Imaging mass cytometry (IMC) was performed on a 5 μm section of the same TMA described above. The section was baked at 60° C. for 90 minutes on a hot plate, de-waxed for 20 minutes in xylene and rehydrated in a graded series of alcohol (100%, 95%, 80% and 70%) for 5 minutes each. Heat-induced antigen retrieval was conducted using a Sous-Vide cooker at 95° C. in Tris-EDTA buffer at pH 9 for 30 minutes. After blocking with 3% BSA in PBS for 45 minutes, the section was incubated overnight at 4° C. with a cocktail of 35 antibodies tagged with rare lanthanide isotopes. The section was counterstained the next day for 40 minutes with iridium (Ir) nuclear stain. Slides were imaged using the Fluidigm Hyperion IMC system with a lum laser ablation spot size and frequency of 200 Hz. Tissue areas of an entire section of each TMA core (approximately 1 mm² per sample), were ablated and imaged. Duplicate cores of the same samples were ablated when morphologic heterogeneity was identified a priori on H&E. Image analyses were performed using CellProfiler (v4.1.3), Ilastik (v1.3.3) and HistoCAT (v1.75). To perform IMC spatial analysis, we selected specific cell types based on marker expression, the number of nearest neighbors, and a spatial interaction range. The spatial interaction range is the distance within which cells are likely to interact, and we chose a range of 50 microns. For each cell, we calculated the spatial interaction score, called ‘spatial score’, to a given cell type as the average distance to the 5 nearest neighbor cells, capped at the spatial interaction range, scaled, and inverted. To ensure the reliability and comparability of spatial scores at the sample level, we calculated the mean of the top 10% of spatial scores of each variable for each sample. By employing this approach, we minimized the bias that zero values of the spatial scores might cause for subsequent analysis at the sample level.

### Prognostic Model Development

To develop a prognostic model for r/r CHL patients, we applied our new cross-format LASSO_plus algorithm (github.com/ajiangsfu/csmpv) on the combination of two separate lists of standardized ‘spatial scores’ and standardized traditional protein-based cellular abundance percentages. LASSO_plus, which draws upon the principles of the well-established LASSO method and incorporates single and stepwise variable selection techniques, allowed us to select variables from two separate lists (58 standardized spatial score variables and 61 standardized protein variables). We initially set the top N=10 for each list, then combined them, ran a Cox model and processed stepwise variable selection on the resulting combined list. We retained variables that were selected from only one data format, and for duplicated variables from both formats, we only kept those with p-values <=0.05. As a result, we obtained a list of six variables (FIG. 14A). To make the model more practical, we manually removed two variables whose end of hazard ratios' 95% confidence intervals (CI) is overlapped with the range of 0.95-1.05 (GzMB pDC spatial score or TIM3 Treg spatial score). Hazard ratio is a ratio based on the risk/survival. This left us with four variables: CXCR5+ HRS spatial score, PD1+CD4 T cell spatial score, Mac (macrophage) spatial score, and CXCR5+ B cell spatial score. We chose to use only the spatial score data format. We used our new algorithm Xgpred in csmpv R package (github.com/ajiangsfu/csmpv) to build the model, which first built an XGBoost (eXtreme Gradient Boosting) model using the four selected variables to predict the post-ASCT outcome. To achieve stable high and low-risk groups based on the XGBoost model score, we applied model-based clustering techniques, and implemented additional filtering steps. After obtaining stable high and low-risk samples, we used known classifications to build a linear regression for each of the four variables to obtain t-values, which were treated as variable weights together with the four variables to build the predictive model with a Linear Prediction Score (LPS), called RHL4S. Finally, we utilized an empirical Bayesian approach to calculate the probability of a sample being classified as a high-risk sample. Based on a probability cut-off of 0.8, any sample with a probability of being in a high-risk group of >=0.8 was classified as high-risk, and the remaining samples were classified as low-risk. XGpred.predict function in the same package can be used for prediction on a new data set when comparable data is available.

### Multi-Color Immunofluorescence (MC-IF) on TMA, Scanning and Image Analysis

For the validation cohort, TMA slides (representing n=40) or individual unstained slides (n=4) were deparaffinized in xylene and rinsed with dH2O. Antigen retrieval was performed in AR6 buffer (PerkinElmer, USA) with Diva decloaker (Biocare Medical, USA). The primary antibody for CXCL13 was incubated for 30 min in an Intellipath FLX rack at room temperature, followed by detection using the Mach2 mouse HRP with 10 min incubation. Visualization of CXCL13 was achieved using Opal 520. The slide was placed into AR6 buffer and heated using a microwave. In serial order, the slide was incubated with primary antibody for CXCR5, followed by detection using Mach2 rabbit HRP, and visualization was accomplished using Opal 650. The slide was again placed into AR6 buffer and heated using a microwave. Then the primary antibody for CD68 was incubated, followed by detection of Mach2 rabbit HRP and Opal 550 for visualization. The slide was placed into AR6 buffer for microwaving. The primary antibody for CD4 was incubated, followed by detection of Mach2 rabbit HRP and visualization for Opal 650. Microwave heating was repeated again with AR6 buffer. The primary antibody for PD-1 was incubated for 30 min in an Intellipath FLX rack at room temperature, followed by detection using the Mach2 mouse HRP with 10 min incubation. Visualization of PD-1 was achieved using Opal 620. The primary antibody for CD30 was incubated, followed by detection of Mach2 mouse HRP and visualization for Opal 570. Nuclei were visualized with DAPI staining and the section was coverslipped using Fluoro Care Anti-Fade Mountant. Entire TMA slides or complete tissue sections were scanned using the Vectra multispectral imaging system (PerkinElmer, USA) following manufacturer's instructions to generate.im3 image cubes for downstream analysis. Optimal exposure times for fluorophores ranged between 50 and 200 ms. To analyze the spectra for all fluorophores included, inForm image analysis software (v2.4.4; PerkinElmer, USA) was used. Cells were first classified into tissue categories using DAPI and CD30 to identify CD30 DAPI, CD30-DAPI, and CD30-DAPI-areas via manual circling and training⁶. The CD30 DAPI regions were considered to be HRS-surrounding regions. Cells were then phenotyped as positive or negative for each of the six markers (CXCL13, CXCR5, CD68, CD4, CD30, PD-1) or (CD20, CXCR5, CD68, CD4, CD30, PD-1). Data were merged in R by X-Y coordinates so that each cell could be assessed for all markers simultaneously. Nearest neighbor analysis was performed with the spatstat R package (v1.58-2).

### Survival Analysis

Overall survival (OS) was defined as the time from diagnosis to death from any cause. Time to first relapse was defined as the time from primary diagnosis to first CHL progression, or death from CHL. Post-ASCT-OS was defined as time from ASCT treatment to death from any cause. Post-ASCT-FFS was defined as time from ASCT treatment to further CHL progression/relapse, or death from any cause. Patients with complete or partial response after second-line chemotherapy were classified as chemo-sensitive. Patients with stable or progressive disease were classified as chemo-resistant. Non-parametric survival analyses with a single binary predictor were analyzed using the Kaplan-Meier method and results were compared using the log rank test. Univariate and multivariate Cox regression analyses were performed to assess the effects of prognostic factors. Survival analyses were performed in the R statistical environment (v4.2.2).

### RHL4S Packages

Since our goal is to establish prognostic model, which can be readily generalized and applicable to patients in daily clinical practice, we translated the findings from IMC into more simplified data. For this aim, we developed the RHL4S R package (github.com/ajiangsfu/RHL4S). The function, RHL4S calls is a wrap-up function designed to process MC-IF data, calculate and calibrate the RHL4S model scores, and classifies patients as either high or low risk for each patient.

### Single Cell RNA Sequencing Sample Preparation

To purify HRS cells, we implemented a flow cytometry-based cell sorting approach. Cell suspensions from CHL tumors were rapidly defrosted at 37° C., washed in RPMI1640/20% FBS solution containing Dnase I (Millipore Sigma, Darmstadt, Germany) and washed in PBS containing 2% FBS. Cells were resuspended in PBC containing 2% FBS and stained with antibody panel specific to isolate HRS cells for 15 minutes at 4° C. in the dark.

Panel A

| Antigen | Antibody Clone | Manufacturer | Catalog number |
|---|---|---|---|
| CD30 | CON6D/B5 | Biocare | CM346 |
| CD4 | EPR6855 | Abcam | Ab133616 |
| CD68 | SP251 | Abcam | Ab192847 |
| CXCR5 | EPR23463-30 | Abcam | Ab254415 |
| CD20 | L26 | Dako | M0755 |
| PD-1 | NAT105 | Cell Marque | 315M-94 |

Panel B

| Antigen | Antibody Clone | Manufacturer | Catalog number |
|---|---|---|---|
| CD30 | CON6D/B5 | Biocare | CM346 |
| CD4 | EPR6855 | Abcam | Ab133616 |
| CD68 | SP251 | Abcam | Ab192847 |
| CXCR5 | EPR23463-30 | Abcam | Ab254415 |
| CXCL13 | 53610 | R & D system | MAB801 |
| PD-1 | NAT105 | Cell Marque | 315M-94 |

Viable cells (DAPI negative) were sorted on a FACS ARIAIII or FACS Fusion (BD Biosciences) using a 130 μm nozzle and were analyzed using FlowJo software (v10.2; TreeStar, Ashland, OR, USA). Sorted cells were collected in 0.3 mL of medium, centrifuged and diluted in 1×PBS with 0.04% bovine serum albumin (BSA). Cell number was determined using a Countess II Automated Cell Counter whenever possible. Enriched HRS cells were loaded into a Chromium Single Cell 5′ Chip kit v2 (PN-120236). Libraries were constructed using the Single 5′ Library and Gel Bead Kit v2 (PN-120237) and Chromium i7 Mulitiplex Kit v2 (PN-120236). The library was measured using Agilent Bioanalyzer High Sensitivity chip and Qubit dsDNA HS Assay Kit.

### Hybrid Capture Sequencing of Marker Genes Using CapID

To boost transcriptome information for cell-to-cell interaction analyses, Hybrid capture sequencing was performed. In brief, Probes for 177 genes were designed and synthesized by Twist Bioscience. Hybridization capture of DNA libraries was performed using Twist Hybridization and Wash Kit (Twist Bioscience). First, we pooled 500 ng of each library to multiplex 8 libraries in a low-bind tube and performed hybridization according to the Twist Target Enrichment Protocol. The captured library was measured using Agilent Bioanalyzer High Sensitivity chip and Qubit dsDNA HS Assay Kit and run on Illumina Nextseq550.

### Normalization and Batch Correction

Analysis and visualization of scRNA-seq data was performed in the R statistical environment (v4.1.0). CellRanger software (v6.0.2) was used to align the sequencing reads to the hg38 human reference genome build. CellRanger (v6.0.2) count data from all cells (n=3302) were read into a single ‘Seurat’ object using the Seurat Package (v4.2.1). Cells were filtered if they had >20% reads aligning to mitochondrial genes, or if their total number of feature counts was less than 1000. This yielded a total of 3302 cells for analysis. The read count matrix was used as input into the “NormalizeData” function which returned a normalized expression matrix. Principal component analysis (PCA) was then run on the normalized expression matrix using highly variable genes identified by “FindVariableGenes” function.

### Clustering and Annotation

Unsupervised clustering was performed with the “FindClusters” function, using the first 30 PCA components as input. Clusters were manually assigned to a cell type by comparing the mean expression of known markers across cells in a cluster. Markers used to annotate cells included CD19 (B cells), CD8, CD3, CD4 (T cells), CD68 (Macrophages) and CCL17, TNFRSF8 (HRS cells). The clustering results were shown in UMAP space which was generated using the first 30 PCA components.

### Cell to Cell Interaction Analysis

Cell Chat analysis. The CellChat R package (v1.1.3) was used to identify potential cell-cell communication networks from scRNA-seq data. Cells were classified into broad subtypes based on their cluster assignments (i.e. HRS-C1, HRS-C2 and etc.) which were input into CellChat as cell labels. The ligand-receptor interaction database (n=1999) used for predicting intercellular communications was the CellChat built-in cross-referencing ligand-receptor interaction database (n=1939) and manually curated ligand-receptor interactions (n=60) which were important in CHL TME biology. The communication probability was calculated for each ligand-receptor pair and the significant interactions were identified (P-values <0.05). The CellChat results were visualized using the “LRPlot” from the iTALK R package (v0.1.0).

### RHL30 Prognostic Model/Assay

RHL30 scores were calculated using methods described in Chan et al. In brief, RNA extracted from formalin-fixed paraffin-embedded tissue (FFPET) were hybridized to RHL30 CodeSet for 12-30 h at 65° C. The RHL30 is a 30 probe NanoString codeset comprising of 18 endogenous genes and 12 housekeepers. The samples were then run on an nCounter Digital Analyzer (Nanostring, Seattle, WA, USA). Then, quality control was performed, and gene expression data were normalized and RHL30 scores were calculated using the same methods. We used the median as a cut-off to distinguish cases with low and high-risk since the original cut-off was not suitable due to batch effects.

### Statistical Results & Visualization

All t-tests reported are two-sided Student's t-tests, and P-values <0.05 were considered statistically significant. In all boxplots, boxes represent the interquartile range with a horizontal line indicating the median value. Whiskers extend to the farthest data point within a maximum of 1.5×the interquartile range, and colored dots represent outliers.

### Data Availability

Single cell RNA-seq counts (generated with CellRanger v2.1.0) and a merged ‘SingleCellExperiment’ R object is available in the European Genome-phenome Archive (EGA) (EGAD00001010892) via controlled access. IMC data is available at zenodo.org/deposit/7963681.

### Code Availability

R packages, csmpv (github.com/ajiangsfu/csmpv) and RHL4S (github.com/ajiangsfu/RHL4S) are available on GitHub.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

What is claimed is:

1. A method of providing an enrichment score for a target cell in a tissue sample, the enrichment score representing enrichment of a selected cell type around the target cell, the method comprising:
a. measuring a distance from the target cell to its nearest cell or to each one of its nearest two or more cells of the selected cell type within a predetermined radius, optionally via an image of the tissue sample;
b. scaling the measured distance via division by the predetermined radius, thereby obtaining a scaled distance, and if the measuring in step a comprises measuring the distance to each one of the nearest two or more cells, then further averaging the scaled distance to obtain an average scaled distance; and
c. performing an inverse operation on the scaled distance or the average scaled distance, thereby obtaining an inverted scaled distance from the target cell to its nearest cell or nearest two or more cells of the selected cell type as the enrichment score for the target cell; and
d. optionally displaying, on a computer screen, the enrichment score based on the inverted scaled distance.

2. The method of claim 1, wherein the tissue sample comprises a quantity of discretely located cells of a same cell type as the target cells, and the method further comprises repeating steps a-c to calculate an enrichment score for each one of the target cells.

3. The method of claim 1, wherein the tissue sample in the image comprises two or more different cell types around the target cell, and the method further comprises repeating steps a-c to calculate an enrichment score representing enrichment of each one of the two or more different cell types around the target cell.

4. The method of claim 1, wherein the target cell is of a same cell type as the selected cell type, thereby the enrichment score representing clustering of the same target cell type.

5. The method of claim 4, wherein the tissue sample comprises two or more different cell types, and the method further comprises repeating steps a-c to calculate an enrichment score representing clustering of each one of the two or more different cell types.

6. The method of claim 1, wherein the target cell is a tumor cell, the tissue sample has a quantity of the tumor cell, and the tissue sample is obtained from a subject with classical Hodgkin lymphoma; and wherein the method further comprises repeating steps a-c to obtain an enrichment score for each tumor cell; optionally wherein the selected cell type comprises PD-1+CD4+ T cell, CD68+ macrophage, CXCR5+ B cell, or CXCR5+ tumor cell.

7. The method of claim 5, further comprising applying a K-means clustering model by a processor based on data records containing the enrichment score for each of the different cell types to generate output data defining niches of the different cell types in the tissue sample; optionally wherein the data records exclude enrichment score greater than a first selected cutoff value, the data records exclude enrichment score smaller than a second selected cutoff value, or the data records exclude enrichment score greater than the first selected cutoff value and enrichment score smaller than the second selected cutoff value, wherein the second selected cutoff value is smaller than the first selected cutoff value.

8. The method of claim 1, wherein the nearest two or more cells comprise about five nearest cells of the selected cell type to the target cell, and the predetermined radius is about 50 μm.

9. The method of claim 1, wherein the inverse operation comprises subtracting the scaled distance or the average scaled distance from a fixed number, optionally the fixed number being 1 or 100%.

10. The method of claim 1, wherein the inverse operation comprises dividing a fixed number by the scaled distance or the average scaled distance, optionally the inverse operation being configured for calculating a multiplicative inverse of the scaled distance or the average scaled distance.

11. The method of claim 1, wherein the measurement is performed on a mass cytometry image, a multicolor immunofluorescence image, or an immunohistochemical stained image, of the tissue sample.

12. The method of claim 1, further comprising one or more of:
i) measuring expression level of a first marker protein in the target cell, optionally further deriving a mathematical relation of the expression level of the first marker protein as a function of the total cell number of the selected cell type within the predetermined radius;
ii) measuring expression level of a second marker protein in the selected cell type within the predetermined radius; and
iii) computing a mathematical product of the first marker protein expression level in the target cell and the second marker protein expression level in the selected cell type.

13. The method of claim 12, wherein the tissue sample has been treated with a panel of labeled antibodies against at least 5, 10, 20, 30, 40, 50, or more marker proteins in the tissue sample, and the method further comprises measuring label intensities of one or more of the at least 5, 10, 20, 30, 40, 50, or more marker proteins in the tissue sample.

14. A system for performing spatial metric analysis including calculating an enrichment score representing enrichment of a selected cell type around a target cell in a tissue sample, the system comprising:
a processor operable to execute computer executable instructions;
a memory operable to store computer executable instructions executable by the processor; and
computer executable instructions stored in the memory and executable to perform the steps in the method of claim 1.

15. A non-transient computer readable medium, comprising:
computer executable instructions, recorded on the non-transient computer readable medium, executable by a processor, for performing the steps in the method of claim 1 to perform spatial metric analysis including calculating an enrichment score representing enrichment of a selected cell type around a target cell in a tissue sample.

16. A method for treating refractory or relapsed classical Hodgkin lymphoma (cHL) in a human subject, the method comprising:
providing a salvage therapy comprising autologous stem cell transplantation (ASCT) or a combination of high-dose chemotherapy and the ASCT to the human subject if the human subject is detected in a biopsy sample of the human subject with presence of enrichment of CXCR5+ B cells around a Hodgkin and Reed Sternberg (HRS) tumor cell and with absence of CXCR5+ HRS tumor cells and absence of enrichment of CXCL13+ macrophages or PD-1+CD4+ T cells around the CXCR5+ HRS tumor cells; or
providing allogeneic bone marrow transplantation, a CD30 targeting treatment, and/or brentuximab vedotin to the human subject if the human subject is detected in the biopsy sample with presence of the CXCR5+ HRS tumor cells and enrichment of the CXCL13+ macrophages and/or PD-1+CD4+ T cells around the CXCR5+ HRS tumor cells;
wherein the enrichment of CXCR5+ B cells comprises two or more CXCR5+ B cells within a radius of no more than about 50 μm from the HRS tumor cell, and the enrichment of CXCL13+ macrophages and/or PD-1+CD4+ T cells comprises two or more of CXCL13+ macrophages and/or PD-1+CD4+ T cells within the radius from the CXCR5+ HRS tumor cells; and
wherein the high-dose chemotherapy comprises a higher dose of chemotherapy than that of a prior chemotherapy to which the cHL is refractory or has relapsed.

17. A method for treating a refractory or relapsed classical Hodgkin lymphoma (r/r cHL) in a human subject, the method comprising:
(a) obtaining two or more enrichment scores for a target cell (also called a home cell) in a biopsy sample from the human subject, each enrichment score representing enrichment of a selected cell type (also called an enriching cell type) around the target cell (also called home cell), wherein the target cell (that is, the home cell) comprises a Hodgkin and Reed Sternberg (HRS) tumor cell, and wherein the selected cell types (that is, the enriching cell types) comprise CXCR5+ HRS tumor cells, PD1+CD4+ T cells, CD68+ macrophages, and CXCR5+ B cells, and wherein each enrichment score is an inverse of an average of scaled distances from the target cell (that is, the home cell) to its nearest two or more cells of respective selected cell type within a predetermined radius, such that the inverse results in a greater enrichment score for a smaller averaged scaled distance compared to that for a larger averaged scaled distance;
(b) calculating a linear predictor score (LPS) for the biopsy sample, wherein each LPS is a linear, weighted combination of the enrichment scores representing enrichment of the four different selected cell types calculated from (a), using an equation:
$$LPS ( X ) = {\sum }_{j} {a}_{j} {X}_{j}$$
wherein Xⱼ is the enrichment score representing enrichment of a selected cell type j around the target cell or is a mean, median or average of top 10%, 20%, 30%, 40%, 50%, 60%, 70%, 80%, or 90% of the highest enrichment scores calculated for a plurality of the target cell type in the biopsy sample; and aⱼ is a scaling factor or weight for the selected cell type j, optionally aⱼ being within 0 to 1; and
(c) calculating a probability that the human subject is at low-risk or at high-risk of salvage treatment failure, optionally salvage treatment failure comprising further relapse, using an equation:
$$P ( X ) = \frac{ϕ ( LPS ( X ) ; {μ}_{1} , {σ}_{1}^{2} )}{ϕ ( LPS ( X ) ; {μ}_{1} , {σ}_{1}^{2} ) + ϕ ( LPS ( X ) ; {μ}_{2} , {σ}_{2}^{2} )} ,$$
wherein:
for calculating the probability of low-risk of salvage treatment failure, Ø(LPS(X); μ₁, σ₁²) is a Phi value when the calculated LPS from step (b) is applied in a normal distribution function with mean μ₁ and variance σ₁² from a first r/r cHL patient population known to have no salvage therapy failure or known with low-risk of salvage therapy failure, and Ø(LPS(X); μ₂, σ₂²) is a Phi value when the calculated LPS from step (b) is applied in a normal distribution function with mean μ₂ and variance σ₂² from a second r/r cHL patient population with known salvage therapy failure or known high-risk of salvage therapy failure; or
for calculating the probability of high-risk of salvage treatment failure, Ø(LPS(X);μ₁,σ₁²) is a Phi value when the calculated LPS from step (b) is applied in a normal distribution function with mean μ₁ and variance σ₁² from the second r/r cHL patient population with known salvage therapy failure or known high-risk of salvage therapy failure, and Ø(LPS(X); μ₂, σ₂²) is a Phi value when the calculated LPS from step (b) is applied in a normal distribution function with mean μ₂ and variance σ₂² from the first r/r cHL patient population known to have no salvage therapy failure or known with low-risk of salvage therapy failure;
(d) classifying the human subject as at low-risk of salvage treatment failure if:
the calculated probability of low-risk of salvage treatment failure from step (c) is 0.8 or greater, or
the calculated probability of high-risk of salvage treatment failure from step (c) is less than 0.5, less than 0.4, less than 0.3, or less than 0.2;
or
classifying the human subject as at high-risk of salvage treatment failure if:
the calculated probability of high-risk of salvage treatment failure from step (c) is 0.8 or greater, or
the calculated probability of low-risk of salvage treatment failure from step (c) is less than 0.5, less than 0.4, less than 0.3, or less than 0.2; and
(e) providing treatment to the human subject, wherein the treatment comprises salvage therapy comprising autologous stem cell transplantation (ASCT) or a combination of high-dose chemotherapy and the ASCT if the human subject is indicated as at low-risk of salvage treatment failure, or wherein the treatment comprises allogeneic bone marrow transplantation, a CD30 targeting treatment, and/or brentuximab vedotin if the human subject is indicated as at high-risk of the salvage treatment failure.

18. The method of claim 17, wherein the scaling factor or weight is a t-value derived from t-statistic of a generalized linear model of binary high/low risk stratification as a function of the enrichment score Xⱼ.

19. A method of treating a patient with Hodgkin's lymphoma, comprising:
administering a salvage therapy optionally a high dose chemotherapy to a patient who is detected with a lower enrichment score according to the method of claim 1 for tumor cells enriched with rosetting cells of CD8+ T cells and/or B cells in an image of a cancer tissue sample obtained from the patient, relative to that of a control subject who has relapsed later than 1 year or has no relapse,
who is detected with a co-expression pattern of marker proteins in the target tumor cell and in the rosetting cells, the co-expression pattern comprising:
the target tumor cell being positive for inducible costimulatory ligand (ICOSL) and rosetting macrophages being positive for inducible T cell co-stimulator (ICOS),
the target tumor cell being positive for galectin-9 and rosetting B cells and/or rosetting macrophages being positive for T cell immunoglobulin and mucin domain-containing protein 3 (TIM3), and/or
the target tumor cell being positive for galectin-9 and rosetting CD4+ T cells, rosetting CD8+ T cells, rosetting B cells, and/or rosetting macrophages being positive for V-domain Ig suppressor of T cell activation (VISTA), and/or
who is detected with a higher CXCR5 expression level in the target tumor cell and/or in the rosetting cells in the cancer tissue sample obtained from the patient, relative to that of the control subject who has relapsed later than 1 year or has no relapse,
optionally with understanding that any one or more of said detections indicates that the patient is likely to have poor outcome or early relapse within 1 year from initial treatment against the Hodgkin's lymphoma.

20. A method of treating a patient with ovarian cancer, comprising:
administering a therapy optionally chemotherapy against the ovarian cancer to a patient who is detected with a higher enrichment score according to the method of claim 1 for stromal cells surrounded by a same type of stromal cells, optionally the stromal cell being an immune cell or podoplanin-positive fibroblast, based on an image of a cancer tissue sample obtained from the patient, relative to that of a control subject who has relapsed after 15 months following debulking surgery for ovarian cancer, and/or
who is detected with a higher percentage of B cells in a region with the higher enrichment score for the fibroblasts in the cancer tissue sample obtained at primary tumor stage from the patient, relative to that in a cancer tissue sample obtained at tumor recurrence stage from the patient,
optionally with understanding that either or both of said detections indicates the patient is likely to have relapsed ovarian cancer within 15 months following a debulking surgery for the ovarian cancer.
